(19) United States
(12) Patent Application Publication
(10) Pub. No.: US 2025/0312430 A1
(43) Pub. Date: Oct. 9, 2025

(54) COMPOSITIONS OF LIPID NANOPARTICLES FOR PLASMID DNA DELIVERY TO THE LIVER AND METHODS FOR PREPARING THE SAME

(71) Applicant: The Johns Hopkins University, Baltimore, MD (US)

(72) Inventors: Hai-Quan MAO, Baltimore, MD (US); Yining ZHU, Baltimore, MD (US); Sashank REDDY, Baltimore, MD (US); Jingyao MA, Baltimore, MD (US); Ruochen SHEN, Baltimore, MD (US); Ivan VUONG, Baltimore, MD (US); Leonardo CHENG, Baltimore, MD (US)

(21) Appl. No.: 18/849,167

(22) PCT Filed: Mar. 30, 2023
(86) PCT No.: PCT/US2023/016938

§ 371 Date: Sep. 20, 2024

Related U.S. Application Data
(60) Provisional application No. 63/325,222, filed on Mar. 30, 2022.

(51) Int. Cl. A61K 39/00; A61K 9/1272; A61K 9/1277; A61K 9/51; A61K 35/00; A61K 39/215; A61P 31/14; C07K 16/28; C12N 7/00; C12N 15/113; C12N 15/88

(52) U.S. Cl. CPC A61K 39/001156; A61K 9/1272; A61K 9/1277; A61K 9/5123; A61K 9/5192; A61K 35/00; A61K 39/215; A61P 31/14; C07K 16/2818; C07K 16/2827; C12N 7/00; C12N 15/113; C12N 15/88; A61K 2039/51; A61K 2039/55555; A61K 2039/57; C12N 2310/14; C12N 2320/31; C12N 2320/32; C12N 2770/20034

(57) ABSTRACT

Lipid nanoparticle formulations with cell type specific transfection activity and capable of producing Th1 and/or Th2 response in vivo and their use for plasmid DNA or mRNA delivery is disclosed.

### STATEMENT OF GOVERNMENTAL INTEREST

This invention was made with government support under grant AI155313 and EB028239 awarded by the National Institutes of Health. The government has certain rights in the invention.

### BACKGROUND

Development of delivery systems and methods remain the most important challenge in realizing the tremendous potential of delivering nucleic acids for gene therapy. RNA- and DNA-based biologics have expansive capacities to modulate cellular activities for treating inherited and acquired diseases. Mulligan, 1993. Among the non-viral gene delivery vectors, the clinical success of LNPs has gained recent widespread attention. Witzigmann et al., 2020; Cullis and Hope, 2017. This is highlighted by the US Food and Drug Administration (FDA)-approved short interfering RNA therapy for hereditary amyloidosis (ONPATTRO®, patisiran) and the two mRNA COVID-19 vaccines approved or authorized for emergency use by millions of healthy people during the pandemic. Adams et al., 2018; Akinc et al., 2019; Hou et al., 2021.

Most lipid-based nucleic acid delivery platforms that are undergoing clinical studies or on the market consist of four or five components: an ionizable lipid, cholesterol, a PEGylated lipid, a helper phospholipid (e.g., 1,2-distearoyl-sn-glycero-3-phosphocholine (DSPC)), and a selective organ targeting lipid. Cheng et al., 2020; Zhang et al., 2021; Wang et al., 2015; Cheng and Lee, 2016. Recent studies have reported that not only the choice of lipid components, but also the relative proportions of the lipid ingredients in the formulation, greatly influence in vivo transfection efficiency and tissue-specific delivery. Cheng et al., 2020; Wei et al., 2020; Oberli et al., 2017; Li et al., 2015; Lokugamage et al., 2021.

Despite the validated ability of these formulations to encapsulate mRNA or siRNA and mediate cellular uptake and endosomal escape, there is a lack of in-depth analysis on the effect of helper lipid charge and the relative ratios of the LNP components on the transfection efficiency for plasmid DNA (pDNA) delivery, which can provide prolonged transgene expression compared to mRNA. Kulkarni et al., 2017; Handumrongkul et al., 2019; Buck et al., 2019; Scholz and Wagner, 2012. In addition, the large number of candidate formulations for screening LNP systems for effective in vivo delivery makes it hard to rationally determine the optimal formulation for particular tissue or disease targets.

### SUMMARY

In some aspects, the presently disclosed subject matter provides a solid nanoparticle comprising a steroid, an ionizable cationic lipid, a helper lipid, a PEGylated lipid, and a nucleic acid payload comprising one or more nucleic acids, wherein the nanoparticle comprises: a molar ratio of the steroid to the PEGylated lipid of between about 10 and about 900; a molar ratio of the ionizable cationic lipid to the helper lipid of between about 1 and about 200; a total percentage of the ionizable lipid and the helper lipid between about 20% and about 80%; and an N to P ratio between about 2 and about 14.

In certain aspects, the steroid comprises a sterol. In particular aspects, the sterol comprises cholesterol.

In certain aspects, the ionizable cationic lipid comprises Dlin-MC3-DMA.

In certain aspects, the helper lipid is selected from a cationic lipid, a zwitterionic lipid, and an anionic lipid.

In certain aspects, the cationic lipid is selected from 1,2-dioleoyl-3-trimethylammonium-propane (DOTAP) and dimethyldioctadecyl ammonium (DDAB).

In certain aspects, the zwitterionic lipid is selected from 1,2-dioleoyl-sn-glycero-3-phosphoethanolamine (DOPE), 2-((2,3-bis(oleoyloxy) propyl)dimethylammonio)ethyl ethyl phosphate (DOCPe), and 1,2-distearoyl-sn-glycero-3-phosphocholine (DSPC).

In certain aspects, the anionic lipid comprises a phospholipid. In particular aspects, the phospholipid is selected from 1,2-dimyristoyl-sn-glycero-3-phosphate (14PA) and 1-stearoyl-2-oleoyl-sn-glycero-3-phospho-(1′-rac-glycerol) (18PG).

In particular aspects, the PEGylated lipid comprises dimyristoyl glycerol (DMG)-polyethyleneglycol (PEG) 2000 (DMG-PEG2000).

In particular aspects, the one or more nucleic acids are selected from plasmid DNA (pDNA), mRNA, siRNA, and combinations thereof. In some aspects, the siRNA comprises an anti-inflammatory siRNA.

In other aspects, the presently disclosed subject matter provides a method for delivering one or more nucleic acids to a liver of a subject, the method comprising administering to a subject in need of treatment thereof a solid nanoparticle as disclosed herein.

In certain aspects, the one or more nucleic acids are selected from plasmid DNA (pDNA), siRNA, and combinations thereof. In particular aspects, the one or more nucleic acids comprises a combination of plasmid DNA (pDNA) and siRNA. In more particular aspects, the siRNA comprises an anti-inflammatory siRNA.

In certain aspects, the anti-inflammatory siRNA targets a transcription factor selected from signal transducer and activator of transcription (STAT), and nuclear factor kappa-light-chain-enhancer of activated B cells (NF-κB). In certain aspects, the method further comprises reducing inflammation-induced gene silencing. In certain aspects, an expression duration of the pDNA when co-administered with the anti-inflammatory siRNA is longer than an expression duration of the pDNA when administered alone. In certain aspects, an expression level of the pDNA when co-administered with the anti-inflammatory siRNA substantially similar to an expression level of the pDNA when administered alone.

In certain aspects, the method comprises reducing a level within the liver of one or more of signal transducer and activator of transcription (STAT), nuclear factor kappa-light-chain-enhancer of activated B cells (NF-κB), one or more infiltrating inflammatory monocytes, and one or more apoptotic cells. In particular aspects, the one or more infiltrating inflammatory monocytes are selected from CD45⁺ and CD11b⁻ cells.

In certain aspects, the method comprises treating one or more diseases or disorders of the liver. In particular aspects, the one more diseases or disorders of the liver are selected from a genetic liver disease and an inflammatory liver disease. In more particular aspects, the one or more disease or disorders of the liver is selected from haemophilia B, haemophilia A, ornithine transcarbamylase (OTC) deficiency, phenylketonuria, acute intermittent porphyria, methylmalonic acidemia, familial hypercholesterolemia, Fabry, MPS type VI, Gangliosidosis GM1, Danon disease, GSD1a Von Gierke, Wilson's disease, Crigler-Najjar, primary hyperoxaluria type 1, and combinations thereof.

In certain aspects, the method for delivering the one or more nucleic acids to a liver of a subject is selected from intravenous (i.v.) injection, oral, subcutaneous, and inhalation delivery.

In other aspects, the presently disclosed subject matter provides a method for preparing a presently disclosed solid nanoparticle, the method comprising:
(a) preparing an organic phase by solubilizing a mixture of a steroid, an ionizable cationic lipid, a helper lipid, a PEGylated lipid in a polar, protic solvent at a predetermined molar ratio;
(b) preparing an aqueous phase by dissolving one or more nucleic acids in an aqueous buffer; and
(c) combining the organic phase and the aqueous phase to form the solid nanoparticle.

In particular aspects, the polar, protic solvent is a C₁-C₄ alcohol. In particular aspects, the aqueous buffer comprises a magnesium acetate buffer.

In certain aspects, the method further comprises mixing the organic phase and the aqueous phase in a flash nanocomplexation (FNC) device.

In certain aspects, the method further comprises mixing the organic phase and the aqueous phase at an about 3:1 ratio.

In certain aspects, the method further comprises dialyzing the solid nanoparticle against deionized water.

In some aspects, the presently disclosed subject matter provides a method for stimulating a Type-1 T helper (Th1) and/or a Type-2 T helper (Th2) response in vivo, the method comprising administering a presently disclosed solid nanoparticle.

In certain aspects, the steroid comprises cholesterol; the ionizable cationic lipid comprises DLin-MC3-DMA; the PEGylated lipid comprises DMG-PEG2000; the nucleic acid comprises a mRNA; and the helper lipid is selected from 1,2-dioleoyl-3-trimethylammonium-propane (DOTAP), dimethyldioctadecyl ammonium (DDAB), 1,2-dioleoyl-sn-glycero-3-phosphoethanolamine (DOPE), DSPC, 1,2-dimyristoyl-sn-glycero-3-phosphate (14PA), and 1-stearoyl-2-oleoyl-sn-glycero-3-phospho-(1′-rac-glycerol) (18PG).

In certain aspects, the solid nanoparticle comprises: a combined molar percentage of DLin-MC3-DMA and helper lipid ranging from about 20% to about 80%; a weight ratio of cholesterol to DMG-PEG2000 ranging from about 10 to about 500; a weight ratio of DLin-MC3-DMA to helper lipid ranging from about 1 to about 200; and a molar ratio of chargeable groups in the ionizable lipid to phosphate groups in mRNA (N/P ratio) ranging from about 4 to about 12.

In particular aspects, the solid nanoparticle comprises: (a) about 30 molar % DOPE, about 30 molar % DLin-MC3-DMA, about 40 molar % cholesterol, about 0.40 molar % DMG-PEG2000, and a N/P ratio of about 4; (b) about 7 molar % DSPC, about 70 molar % DLin-MC3-DMA, about 20 molar % cholesterol, about 0.04 molar % DMG-PEG2000, and a N/P ratio of about 4; or (c) about 5 molar % 18PG, about 55 molar % DLin-MC3-DMA, about 40 molar % cholesterol, about 0.40 molar % DMG-PEG2000, and a N/P ratio of about 12.

In certain aspects, the method induces an immune response in Th1 only, in Th2 only, or in both Th1 and Th2.

In other aspects, the presently disclosed subject matter provides a method for treating a disease, disorder, or condition in subject, the method comprising administering a therapeutically effective dose of a presently disclosed solid nanoparticle to a subject in need of treatment thereof.

In certain aspects, the disease is selected from a cancer or an infection. In particular aspects, the cancer is selected from basal cell carcinoma, bladder cancer, breast cancer, cervical cancer, colorectal cancer, endometrial cancer, esophageal carcinoma, gastric cancer, head and neck cancer, hepatocellular carcinoma, Hodgkin's lymphoma, malignant pleural mesothelioma, Merkel cell carcinoma, metastatic melanoma, non-small cell lung cancer, renal cell carcinoma, small cell lung cancer, squamous cell carcinoma, and urothelial carcinoma.

In certain aspects, the infection comprise a viral infection. In particular aspects, the viral infection is selected from a coronavirus infection, a Zika virus infection, influenza, a flavivirus infection, and a human immunodeficiency virus (HIV) infection.

In other aspects, the method further comprises administering the solid nanoparticle with one or more immune checkpoint inhibitors. In certain aspects, the immune checkpoint inhibitor is selected from a CTLA-4 inhibitor, a PD-1 inhibitor, and a PD-L1 inhibitor. In particular aspects, the one or more immune checkpoint inhibitors is selected from Ipilimumab, Nivolumab, Pembrolizumab, Atezolizumab, Avelumab, Durvalumab, and Cemiplimab.

In other aspects, the presently disclosed subject matter provides a vaccine comprising the solid nanoparticle disclosed herein. In certain aspects, the vaccine is a cancer vaccine or an anti-viral vaccine.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawing(s) will be provided by the Office upon request and payment of the necessary fee.

Having thus described the presently disclosed subject matter in general terms, reference will now be made to the accompanying Figures, which are not necessarily drawn to scale, and wherein:

FIG. 1 is a schematic illustration of multi-step composition screening of lipid nanoparticles (LNPs) for liver-targeted pDNA delivery. In vitro transfection efficiency was assessed for 1,080 LNP formulations with different helper lipids and component ratios. The top-performing formulations for each helper lipid series were then tested in clusters for cytotoxicity and in vivo local transfection efficiency via intrahepatic injection. Clusters that induced minimal cytotoxicity and high transfection were screened via i.v. injection, and LNP formulations within the clusters that demonstrated optimal liver transfection were further evaluated individually;

FIG. 2A, FIG. 2B, FIG. 2C, FIG. 2D, FIG. 2E, FIG. 2F, and FIG. 2G shows in vitro LNP-mediated pDNA delivery. (FIG. 2A) Transfection efficiency of LNPs in HepG2 cells via high-throughput screening platform after 72 h incubation (n=2). The level of transgene expression for each formulation is shown using luciferase as a reporter. (FIG. 2B) The top 32 formulations from each helper lipid series were selected based on transfection efficiency in HepG2. (FIG. 2C-FIG. 2E) FACS was used to further evaluate the transfection efficiency of (FIG. 2C) DOTAP, (FIG. 2D) DOPE and (FIG. 2E) 18PG series of LNPs using GFP as a reporter gene at a pDNA dose of 1 μg mL⁻¹; gene expression was analyzed at 72 h after transfection (n=2). Cellular metabolic activity was measured by alamarBlue assay (n=4). Formulations were regrouped into four clusters, each containing eight formulations, based on their transgene expression level. Data are presented as mean±S.D. The percentage of each component in the formulations is indicated by pie charts. See Tables 1-6 for molar percentage used in the 32 formulations for each helper lipid. (FIG. 2F) Histogram of the Z-average diameters of top 32 LNP formulations made from each helper lipid. (FIG. 2G) Percentage of LNP formulations with size less than 200 nm and less than 400 nm for each helper lipid;

FIG. 3A, FIG. 3B, FIG. 3C, FIG. 3D, and FIG. 3E show LNP-mediated local intrahepatic pDNA delivery in cluster-mode screening. (FIG. 3A) Scheme for intrahepatic delivery. (FIG. 3B) Whole-body bioluminescence flux of BALB/c mice at 12 h following a single intrahepatic injection of different LNP formulations containing 3 μg of pDNA (50% Luc+50% mCherry) per mouse (n=2). (FIG. 3C-FIG. 3D) Ex vivo (FIG. 3C) imaging and (FIG. 3D) quantitative flux of luminescence in the liver at 12 h post-administration. (FIG. 3E) FACS was used to quantify the percentage of specific cell types in the liver expressing mCherry for the top 12 clusters. Data are presented as mean±S.D. (n=2 mice per group);

FIG. 4A, FIG. 4B, FIG. 4C, FIG. 4D, FIG. 4E, FIG. 4F, FIG. 4G, FIG. 4H, and FIG. 4I show the in vivo transfection efficiency of LNPs administered via i.v. injection in cluster-mode screening. (FIG. 4A-FIG. 4B) Whole-body bioluminescence (FIG. 4A) quantitative measurement and (FIG. 4B) imaging of BALB/c mice at 12 h after a single i.v. administration of different LNPs formulation containing 100 μg of pDNA (50% Luc+50% mCherry) per mouse (n=2). (FIG. 4C-FIG. 4D) Ex vivo imaging and quantitative luminescence measurement of the liver of BALB/c mice at 12 h post-administration. (FIG. 4E) FACS was used to quantify the percentage of specific cell types within mCherry+ cells in the liver. (FIG. 4F) FACS was used to quantify the percentage of mCherry+ cells within hepatocytes (FSChi SSChi cells in CD45−CD31−CD11b−CD326− cells). (FIG. 4G-FIG. 4H) Quantitative measurement of luminescence and relative luciferase expression in each organ. (FIG. 4I) FACS was used to quantify the percentage of mCherry+ cells within the major organs. Data above are presented as mean±S.D.;

FIG. 5A, FIG. 5B, FIG. 5C, FIG. 5D, FIG. 5E, FIG. 5F, FIG. 5G, FIG. 5H, FIG. 5I, FIG. 5J, FIG. 5K, and FIG. 5L show the liver-targeted transfection efficiency and in vivo gene editing by LNPs administered via i.v. injection. (FIG. 5A-FIG. 5B) Whole-body bioluminescence imaging and quantitative measurement of BALB/c mice at 12 h after a single i.v. injection of different LNP formulations containing 50 μg of pDNA (50% Luc+50% mCherry) per mouse (n=2). (FIG. 5C-FIG. 5D) Ex vivo imaging and quantitative luminescence measurement of the liver of BALB/c mice at 12 h post-administration with single dosage. (FIG. 5E-FIG. 5F) Quantitative measurement of luminescence and relative luciferase expression level in each organ. (FIG. 5G) Schematic illustrating that the delivery of Cre pDNA activates tdTom expression in tdTom transgenic mice via Cre-mediated genetic deletion of the stop cassette. (FIG. 5H) Ex vivo quantitative measurement of tdTom fluorescence in the liver of Ai9 mice at 3 days post-administration with a single dosage of LNPs containing 25 μg of pDNA (Cre) per mouse (i.v., n=2). (FIG. 5I) TdTom expression visualized by confocal imaging of tissue sections. (FIG. 5J) Ex vivo imaging of tdTom fluorescence in the different organs of the Ai9 mice at 3 days post-administration with single dosage. (FIG. 5K) Relative tdTom expression in each organ. (FIG. 5L) FACS was used to quantify the percentage of tdTom+ cells in the liver. All data are presented as mean±S.D.;

FIG. 6A, FIG. 6B, FIG. 6C, FIG. 6D, FIG. 6E, FIG. 6F, FIG. 6G, and FIG. 6H show the biodistribution, cellular uptake and endosomal escape levels of top-performing LNP formulations. (FIG. 6A) Biodistribution at 6, 12 and 24 h post-injection of LNPs (30 μg pDNA (85% Luc+15% Cy5-labeled p1216) per mouse. i.v. n=3). (FIG. 6B) FACS was used to quantify the percentage of Cy5+ hepatocytes in the liver at 6 and 12 h post-injection. (FIG. 6C-FIG. 6D) Ex vivo quantitative measurement and luminescence imaging of the liver of BALB/c mice at 24 h post-administration (25 μg Luc pDNA per mouse, intrahepatic injection, n=3). (FIG. 6E-FIG. 6G) In vitro transfection and cellular uptake of selected formulations on primary hepatocytes. FACS was used to quantify the percentages of (FIG. 6E) Cy5+ cells and (F) GFP+ cells within primary hepatocytes isolated from the liver (1 μg mL⁻¹ pDNA (75% GFP+25% Cy5-labeled p1216)). (FIG. 6G) Representative FACS data for LNPs pre-incubated with mouse serum for 0.5 h at an LNP/serum volume ratio of 2:1 before dosing. (FIG. 6H) Quantitative Cellomics high-content analysis for endosomal escape mediated by LNPs in vitro. Average number of Gal8 spots per cell (B16-Gal8-GFP) at 12 h post-treatment as an indication of endosomal escape level (1 μg mL⁻¹ pDNA). Statistical P-values: No significance: NS; *P<0.05, **P<0.01, ***P<0.001, ****P<0.0001;

FIG. 7A, FIG. 7B, FIG. 7C, FIG. 7D, FIG. 7E, FIG. 7F, and FIG. 7G show the durable expression of pDNA LNPs and extended transgene expression duration by co-delivery of anti-inflammatory siRNA. (FIG. 7A-FIG. 7B) Whole-body bioluminescence imaging of Balb/c mice at different time points after i.v. administration of a single dosage of LNPs containing 25 μg of Luc pDNA per mouse or 5 μg of Luc mRNA per mouse for mRNA LNPs (n=3). (FIG. 7C-FIG. 7E) Whole-body bioluminescence imaging of Balb/c mice at different time points post-administration (25 μg Luc pDNA per mouse, 2.5 μg siRNA for each transcription factor per mouse (n=3). (FIG. 7F) The levels of transcription factors of treated mice were determined by ELISA at 7 days post-administration with single dosage. (FIG. 7G) FACScan was used to determine the infiltrating inflammatory monocytes (CD45⁺CD11b⁺ cells) in the liver after treatments. Data above are presented as mean±S.E.M. Statistical P-values: No significance: NS; *P<0.05, **P<0.01, ***P<0.001, **** P<0.0001. Without specific indications, the label above each group indicates the statistical comparison with the PBS control group;

FIG. 8A, FIG. 8B, and FIG. 8C show the transfection efficiency of Groups B, D, and E LNPs prepared using DDAB, DSPC or 14PA as the helper lipid, respectively. FACS was used to further evaluate the transfection efficiency of (FIG. 8A) DDAB, (FIG. 8B) DSPC and (FIG. 8C) 14PA LNPs (1 μg mL⁻¹ pDNA (GFP), 72 h, n=2). The efficiency of transgene expression of GFP as a reporter. Cellular metabolic activity was measured by alamarBlue assay (n=4). Formulations were regrouped into four clusters, each containing eight formulations, based on their transgene expression level. Data are presented as mean #S.D. The percentage of each component in the formulations is indicated by pie charts. See Tables 1-6 for molar percentages of all lipids used in the 32 formulations in each group of LNPs;

FIG. 9 shows the effect of various formulation parameters on the average size of LNPs. The average sizes and size distributions of the top performing LNPs (Top 32 formulations from each LNP group) were measured using dynamic light scattering (DLS);

FIG. 10 shows the survival of Balb/c mice following a single intravenous injection of different clusters of LNP formulations. The three most toxic clusters (AII, AIV, DIV) were shown in this survival graph, where all other tested clusters did not cause animal death. LNPs were injected at a total pDNA dose of 100 μg (50% Luc+50% mCherry) per mouse for each cluster (n=2);

FIG. 11 shows the survival of Balb/c mice following a single intravenous injection of different LNP formulations. The five most toxic formulations (DI-2, DI-4, FIII-2, FIII-3, FIII-4) were shown in this survival graph, where all other tested formulations did not cause any death. LNPs were injected at a total pDNA dose of 50 μg (50% Luc+50% mCherry) per mouse (n=2);

FIG. 12 shows the average tdTom expression levels in different organs at 3 days after a single i.v. injection of LNPs in Ai9 mice. The dose of Cre pDNA was 25 μg per mouse (i.v., n=2). Data are presented as mean±S.D.;

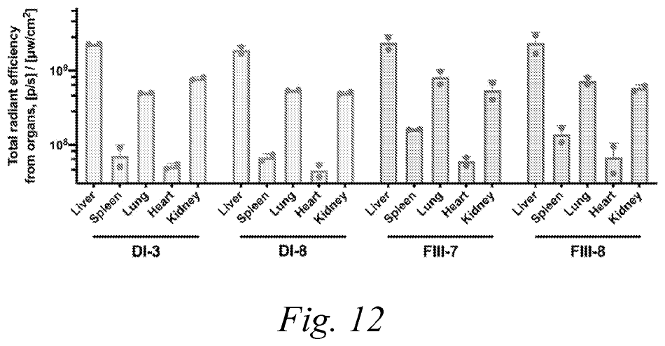

FIG. 13 shows the percentage of tdTom+ cells in the major organs at 3 days after a single i.v. injection of LNPs in Ai9 mice. FACS was used to quantify the percentage of tdTom+ cells in each organ (25 μg Cre pDNA per mouse, i.v., n=2). Data are presented as mean±S.D.;

FIG. 14. Z-average size and zeta potential of different LNP formulations in clusters DI and FIII measured by DLS (n=3). Data are presented as mean±S.D. The percentage of each component in the formulations is indicated by pie charts. See Tables 4 and 6 for molar percentage used in the selected formulations;

FIG. 15 shows the biodistribution in the liver at 6 h, 12 h, and 24 h post-injection of LNPs in different clusters (DI-3, DI-6, DI-8, FIII-1, FIII-7, and FIII-8). Biodistribution in the liver was determined at 6 h, 12 h, and 24 h post-injection of different clusters of LNPs at a total pDNA dose of 30 μg per mouse (85% Luc+15% Cy5-labeled 1216), i.v., n=3);

FIG. 16 shows the percent of apoptotic cells (Zombie Violet-Apotracker Green+ cells) within the liver after one single i.v. injection of the selected LNPs. FACS was used to quantify the percentage of Zombie Violet-Apotracker Green+ cells in the liver (25 μg Luc pDNA per mouse, 2.5 μg siRNA for each transcription factor per mouse, i.v., n=3). Data are presented as mean±S.E.M.;

FIG. 17A, FIG. 17B, FIG. 17C, and FIG. 17D show the effect of several key formulation parameters, including the type of helper lipid, the molar ratio of the ionizable lipid to the helper lipid, the molar ratio of the steroid to the PEGylated lipid, the total percentage of the ionizable lipid and the helper lipid, and the N to P ratio, on the in vitro LNP-mediated pDNA delivery using a high-throughput screening platform after 72 h incubation (n=2). The level of transgene expression for each formulation is shown using luciferase as a reporter. (FIG. 17A) Strip plot of transfection efficiency of 1080 LNPs in B16-F10 melanoma cells grouped by the molar ratio of DLin-MC3-DMA to helper lipid used in the formulation. (FIG. 17B) Strip plot of transfection efficiency of 1080 LNPs in B16-F10 melanoma cells grouped by the molar ratio of cholesterol to DMG-PEG2000 used in the formulation. (FIG. 17C) Strip plot of transfection efficiency of 1080 LNPs in B16-F10 melanoma cells grouped by the total percentage of DLin-MC3-DMA and helper lipid used in the formulation. (FIG. 17D) Strip plot of transfection efficiency of 1080 LNPs in B16-F10 melanoma cells grouped by N and P ratio used in the formulation;

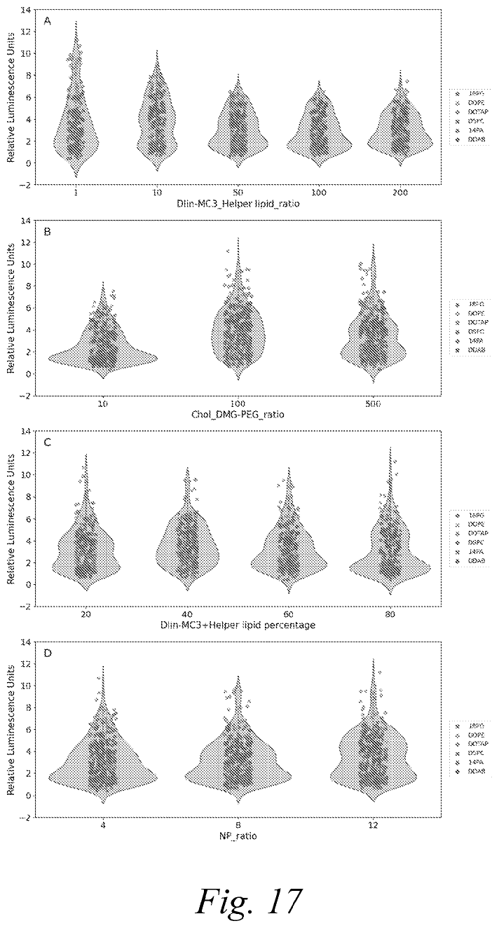

FIG. 18A, FIG. 18B, FIG. 18C, FIG. 18D show the effect of several key formulation parameters, including the molar ratio of the ionizable lipid to the helper lipid, the molar ratio of the steroid to the PEGylated lipid, the total percentage of the ionizable lipid and the helper lipid, and the N to P ratio, on the in vitro LNP-mediated pDNA delivery using a high-throughput screening platform after 72 h incubation (n=2). The level of transgene expression for each formulation is shown using luciferase as a reporter. (FIG. 18A) Strip plot of transfection efficiency of 1080 LNPs in HepG2 hepatocellular carcinoma cells grouped by the molar ratio of DLin-MC3-DMA to helper lipid used in the formulation. (FIG. 18B) Strip plot of transfection efficiency of 1080 LNPs in HepG2 hepatocellular carcinoma cells grouped by the molar ratio of cholesterol to DMG-PEG2000 used in the formulation. (FIG. 18C) Strip plot of transfection efficiency of 1080 LNPs in HepG2 hepatocellular carcinoma cells grouped by the total percentage of DLin-MC3-DMA and helper lipid used in the formulation. (FIG. 18D) Strip plot of transfection efficiency of 1080 LNPs in HepG2 hepatocellular carcinoma cells grouped by N and P ratio used in the formulation;

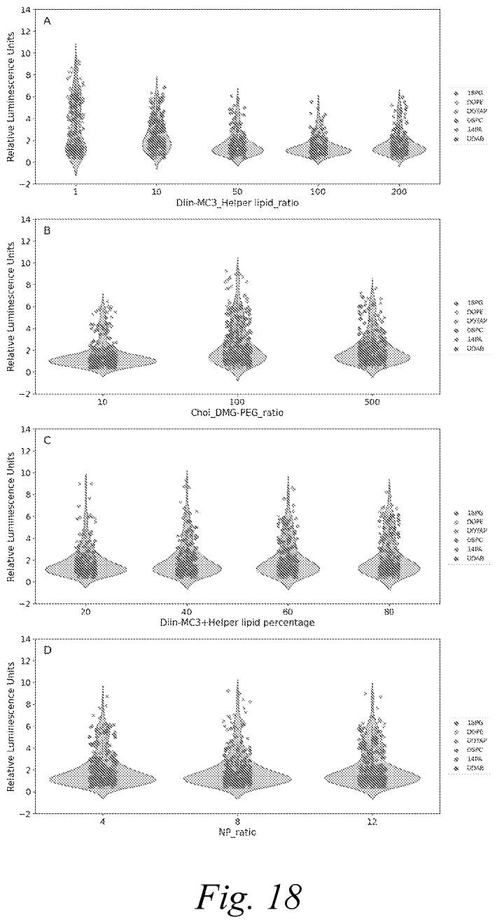

FIG. 19A, FIG. 19B, FIG. 19C, FIG. 19D show the effect of several key formulation parameters, including the type of helper lipid, the molar ratio of the ionizable lipid to the helper lipid, the molar ratio of the steroid to the PEGylated lipid, the total percentage of the ionizable lipid and the helper lipid, and the N to P ratio, on the in vitro LNP-mediated pDNA delivery using a high-throughput screening platform after 72 h incubation (n=2). The level of transgene expression for each formulation is shown using luciferase as a reporter. (FIG. 19A) Strip plot of transfection efficiency of 1080 LNPs in PC3 human prostate cancer cells grouped by the molar ratio of Dlin-MC3-DMA to helper lipid used in the formulation. (FIG. 19B) Strip plot of transfection efficiency of 1080 LNPs in PC3 human prostate cancer cells grouped by the molar ratio of cholesterol to DMG-PEG2000 used in the formulation. (FIG. 19C) Strip plot of transfection efficiency of 1080 LNPs in PC3 human prostate cancer cells grouped by the total percentage of DLin-MC3-DMA and helper lipid used in the formulation. (FIG. 19D) Strip plot of transfection efficiency of 1080 LNPs in PC3 human prostate cancer cells grouped by N and P ratio used in the formulation;

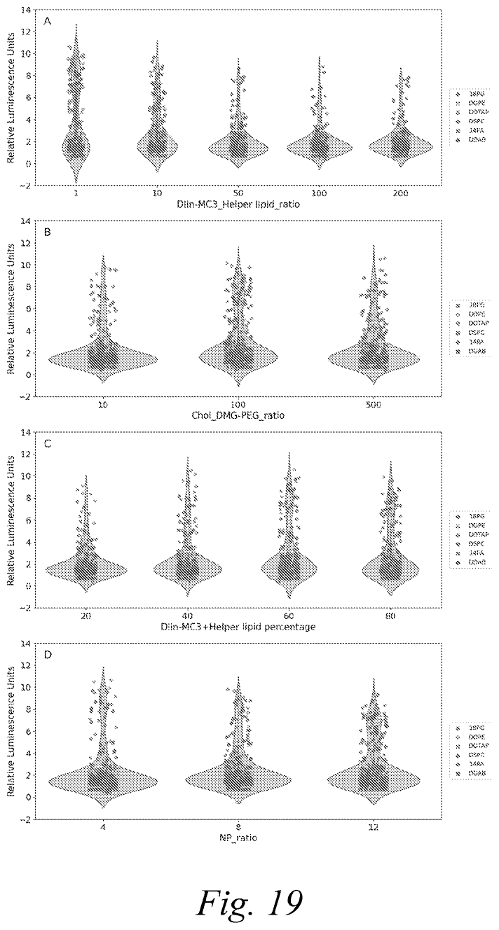

FIG. 20A and FIG. 20B show the in vivo transfection efficiency of the selected LNP formulations administered via intraduodenal injection in Balb/c mice. (FIG. 20A-FIG. 20B) Bioluminescence flux of liver at 48 h post-administration with single dosage. The top six formulations (above the dotted line) were selected. The formulation details of these six formulations are showed in Table 7;

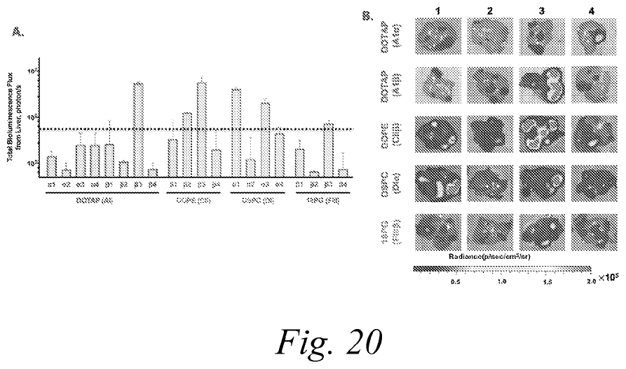

FIG. 21 shows the transfection efficiency of mRNA LNPs on primary neurons. Transfection efficiency of GFP mRNA LNPs in primary neurons was evaluated via flow cytometry after a 24-h transfection (n=3; mRNA 0.5 μg/well). The top eight formulations (A2, A3, A5, B9, C9, D7 D10, F5) were selected for future investigations. The formulation details of these eight formulations are showed in Table 8;

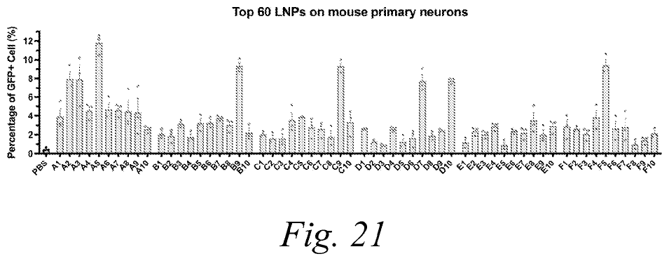

FIG. 22a, FIG. 22b, FIG. 22c, FIG. 22d, FIG. 22e, FIG. 22f, FIG. 22g, and FIG. 22h show in vitro screening of mRNA lipid nanoparticles for transfection and induction of antigen presentation and maturation in DCs. FIG. 22a, Schematic of the screening platform and the therapeutic mechanism of mRNA LNP vaccination against a solid tumor. In vitro transfection efficiency was assessed for 1,080 LNP formulations with different helper lipids and component ratios. The top-performing formulations were then tested on BMDCs for transfection and antigen presentation and in vivo immune responses induced by selected LNPs were assessed. LNPs transfect tissue resident DCs following s.c. injection; or drain into the neighboring lymph nodes, where they transfect APCs including DCs. These APCs translate and process the mRNA into peptides presented on major histocompatibility complex molecules on cell surface. The lipids also trigger activation pathways that promote costimulatory molecule expression and cytokine release. T cells activated by the APCs proliferate and travel to the tumor site to kill cancer cells in an antigen-specific manner. DC, dendritic cell; IFN-γ, interferon-γ; MHC, major histocompatibility complex; TAP, transporter associated with antigen processing; TCR, T cell receptor; TNF-α, tumor necrosis factor α. FIG. 22b, DC 2.4 cells were treated with fLuc mRNA LNPs (1 μg mL⁻¹). The relative luciferase expression after 24 h incubation with fLuc mRNA LNPs is shown in a heat map. FIG. 22c, BMDCs were treated with the 49 top-performing LNPs packaged with mCherry mRNA. The percentage of mCherry⁺ cells gated on CD11c⁺ cells after 24 h incubation with mRNA LNPs is shown. LNP formulation details are shown in pie charts with Dlin-MC3-DMA in red, cholesterol in green, DMG-PEG2000 in yellow, and helper lipids in blue. The top seven formulations, indicated by red arrows, were selected for further study. d-f, Antigen presentation (FIG. 22d), with maturation levels of BMDCs (FIG. 22e, FIG. 22f) were analyzed by flow cytometry after 24 h incubation with the seven OVA mRNA-loaded LNPs, PBS, free OVA, or LPS and SIINFEKL peptide. The percentages of SIINFEKL-H-2Kb⁺ cells (FIG. 22d), additionally positive for CD86 (FIG. 22e) or CD40 (FIG. 22f) gated on CD11c⁺ cells are shown. FIG. 22g, Representative flow cytometry analysis of SIINFEFL-H-2Kb and CD40 expression on BMDCs treated with the three top-performing LNPs. FIG. 22h, Secretion levels of IFN-γ, TNF-α and IL-6 within the supernatant of BMDCs after 24 h incubation with the three OVA mRNA (mOVA)-loaded LNPs were measured by ELISA and are shown in a radar chart. Data represent the mean±s.e.m. from a representative experiment (n=3 for (FIG. 22b-FIG. 22g), n=4 for (FIG. 22h) biologically independent samples) of two independent experiments. Data were analyzed using one-way ANOVA and Dunnett's multiple comparisons test against PBS group. *P<0.05, **P<0.01, ***P<0.001. NS, not significant;

FIG. 23a, FIG. 23b, FIG. 23c, FIG. 23d, FIG. 23e, FIG. 23f, FIG. 23g, FIG. 23h, FIG. 23i, FIG. 23j, FIG. 23k, FIG. 23l, and FIG. 23m show in vivo assessments of lymph node cell transfection and immune activation by top LNP formulations. FIG. 23a, Schematic of the Ai9 mouse model and experiment. Cre recombinase is expressed from exogenously delivered Cre mRNA (mCre) and cleaves the LoxP sites flanking a stop sequence in the mouse genome, enabling expression of the fluorescent tdTomato reporter. FIG. 23b-FIG. 23c, Ai9 mice were administered the top three LNPs loaded with mCre via i.m. and s.c. injections (10 μg mCre per mouse). Transfection of immune cells in draining lymph nodes was analyzed by flow cytometry. Percentages of cells positive for tdTomato (FIG. 23b), as well as CD11c (FIG. 23c) gated on CD45⁺ cells are shown. FIG. 23d, Timeline for the immune activation experiment. C57BL/6 mice were given three s.c. injections, one week apart, of PBS, free OVA protein, or C10, D6, or F5 LNPs loaded with mOVA (10 μg OVA protein or 10 μg mOVA per injection). Mice were sacrificed one week after the final injection, and their splenocytes and lymphocytes were isolated for analysis. FIG. 23e, FIG. 23f, C57BL/6 mice were administered PBS, free OVA protein, or C10, D6, F5 LNPs loaded with mOVA via s.c. injection. DC antigen presentation (FIG. 23e), with maturation levels (FIG. 23f) in the draining lymph nodes were analyzed through flow cytometry. Cells positive for CD11c and SIINFEKL-H-2Kb (FIG. 23e), as well as CD86 (FIG. 23f) are shown. FIG. 23g-FIG. 23k, C57BL/6 mice were administered PBS, free OVA protein, OVA protein mixed with aluminum hydroxide gel (Alhydrogel®) (1:1) or C10, D6, F5 and SM-102 LNPs loaded with mOVA via s.c. injection (10 μg OVA protein or 10 μg mOVA per injection). Splenocytes were restimulated in vitro with OVA and SIINFEKL peptide (100 μg mL⁻¹ OVA and 2 μg mL⁻¹ SIINFEKL) for 6 h and assessed via flow cytometry and intracellular cytokine staining to determine the percentages of CD8⁺IFN-γ+ (FIG. 23g), CD8⁺ granzyme B⁺ (FIG. 23h), CD8⁺ TNF-α⁺ (FIG. 23l), and CD4⁺IFN-γ⁺ (FIG. 23j) cells. FIG. 23k, Frequency of IFN-γ-producing cells among restimulated splenocytes, assessed via ELISPOT. FIG. 23l, Percentage of restimulated splenocytes double-positive for CD4 and IL-4, assessed by flow cytometry and ICS and representing Th2 cells. FIG. 23m, Titer of OVA-specific IgG antibodies in blood serum on day 21, determined by ELISA. Data represent the mean #s.e.m. from a representative experiment (n=4 (FIG. 23b, FIG. 23c, FIG. 23e-FIG. 23m) biologically independent samples) of two independent experiments. Data were analyzed using one-way ANOVA and Dunnett's multiple comparisons test for FIG. 23e-FIG. 23j and FIG. 23l-FIG. 23m, one-way ANOVA and Turkey's multiple comparisons test for FIG. 23b, FIG. 23c, and FIG. 23k. *P<0.05, **P<0.01, ***P<0.001. NS, not significant;

FIG. 24a, FIG. 24b, FIG. 24c, FIG. 24d, FIG. 24e, FIG. 24f, FIG. 24g, FIG. 24h, FIG. 24i, FIG. 24j, FIG. 24k, and FIG. 24l demonstrate anti-tumor efficacy of top mRNA LNP formulations as prophylactic and therapeutic vaccines. FIG. 24a-FIG. 24d, Schematic and results of a prophylactic vaccination model for OVA-expressing melanoma in C57BL/6 mice. Mice were given three s.c. injections (10 μg mOVA per injection), one week apart, of PBS, free OVA protein, OVA protein mixed with Alhydrogel® (1:1) or mOVA-loaded C10, D6, F5, or SM-102 LNPs prior to s.c. inoculation of OVA-expressing melanoma (B16F10-OVA) cells (FIG. 24a). Survival curves (FIG. 24B), average tumor volume (FIG. 24c), and individual tumor volume (FIG. 24d) are shown over. FIG. 24e-FIG. 24h, Schematic and results of a therapeutic vaccination model for B16F10-OVA in C57BL/6 mice. Mice were inoculated s.c. with B16F10-OVA cells and then given three s.c. injections, one week apart, of PBS or mOVA-loaded C10 (10 μg mOVA per injection). Two groups received a repeated anti-CTLA-4 monoclonal antibody (mAb; 100 μg per i.p. injection) treatment alone or in combination with the LNP treatment (FIG. 24e). Survival curves (FIG. 24F), average tumor volume (FIG. 24g), and individual tumor volume (FIG. 24h) are shown over time. FIG. 24i-FIG. 24l, Schematic and results of a therapeutic vaccination model against melanoma-associated antigens for melanoma in C57BL/6 mice. Mice were inoculated s.c. with B16F10 cells and then given three s.c. injections, one week apart, of PBS or C10 LNP loaded with mRNA encoding Trp2 (mTrp) or Gp100 (mGp100) (10 μg mRNA per injection). Two groups received the anti-CTLA-4 mAb (100 μg per i.p. injection) treatment in combination with the LNP treatment (FIG. 24i). Survival curves (FIG. 24j), average tumor volume (FIG. 24k), and individual tumor volume (FIG. 24l) are shown over time. Data represent the mean±s.e.m. from a representative experiment (n=7 (FIG. 24b-FIG. 24d), n=6 (FIG. 24f-FIG. 24h, FIG. 24i-FIG. 24l) biologically independent samples) of two independent experiments. Differences between treatment groups were analyzed using one-way ANOVA and Tukey's multiple comparisons test. Survival curves were compared using log-rank Mantel-Cox test, and the stack of P values were corrected by Holm-Šídák method for multiple comparisons with alpha set to 0.05. *P<0.05, **P<0.01, ***P<0.001. NS, not significant; i.p. intraperitoneal; αCTLA-4, anti-CTLA-4 monoclonal antibody;

FIG. 25a, FIG. 25b, FIG. 25c, FIG. 25d, FIG. 25e, FIG. 25f, FIG. 25g, FIG. 25h, FIG. 25i, and FIG. 25j demonstrate that the coordinated attack by T cells and NK cells was responsible for long-term protection. FIG. 25a-FIG. 25g, Schematic and results of cell depletion experiments in the prophylactic vaccination model for OVA-expressing melanoma in C57BL/6 mice. Mice were given three s.c. injections, one week apart, of PBS or mOVA-loaded C10 or F5 LNPs (10 μg mOVA per injection) prior to s.c. inoculation of B16F10-OVA cells, and antibody for cell depletion were injected every four days (i.p., 200 μg per mice) (FIG. 25a). Survival curves (FIG. 25b, FIG. 25e), average tumor volume (FIG. 25c, FIG. 25f), and individual tumor volume (FIG. 25d, FIG. 25g) are shown over time (n=6 biologically independent mice per group). FIG. 25h, Tumor-infiltrating immune cells including NK cells, T cells, CD8+ T cells and Treg cells were determined by flow cytometry on day 22 post tumor inoculation (n=6 per group). FIG. 25i, Ratio of CD8⁺ T-cell percentage to CD4⁺FoxP3⁺CD25⁺ Treg cell percentage on day 22 post tumor inoculation (n=6 per group). FIG. 25j, Immunofluorescent analysis of CD3 T cell and NK cell infiltration of tumor section on day 22 post tumor inoculation. DAPI (blue), CD3 (green), NK 1.1 (red), scale bar 50 μm. Data were presented as mean±s.e.m. Differences between treatment groups were analyzed using one-way ANOVA and Tukey's multiple comparisons test. Survival curves were compared using log-rank Mantel-Cox test, and the stack of P values were corrected by Holm-Šídák method for multiple comparisons with alpha set to 0.05. *P<0.05, **P<0.01, ***P<0.001. NS, not significant; and

FIG. 26a, FIG. 26b, FIG. 26c, FIG. 26d, FIG. 26e, FIG. 26f, FIG. 26g, FIG. 26h, FIG. 26i, and FIG. 26j show local transfection, cellular uptake, and endosomal escape of mRNA LNPs. FIG. 26a, Schematic of different immune responses induced by mRNA LNPs. Transfected APCs translate, process, and present antigen epitopes on MHC-I molecules to CD8+ T cells, while transfected non-APCs, such as myocytes translate and release antigen for APCs to internalize and present antigen epitopes on MHC-II molecules to helper T cells. Some exogenous antigens were uptaken and presented on MHC-I molecules by cross-presentation pathway. FIG. 26b, FIG. 26c, Makeup of transfected cells at the injection sites at 24 h post-injection with GFP mRNA (mGFP)-loaded C10, D6, and F5 formulations. Flow cytometry was used to determine the ratios of non-immune and immune cells (FIG. 26b) and the relative abundance of each cell type (FIG. 26c). (Immune cells (CD45+), epithelial cells (CD326+), endothelial cells (CD31+), muscle cells (desmin+), adipocytes (CD45-CD31-CD36+)) FIG. 26d-FIG. 26h, In vitro evaluation of transfection or uptake efficiency by formulations C10, D6, and F5 in C2C12 cells and BMDCs. Flow cytometry was used to determine the ratios of mCherry mRNA-transfected C2C12 cells to transfected BMDCs in in vitro co-culture (FIG. 26d), transfection efficiency of LNPs containing fLuc mRNA in pure C2C12 cell culture (FIG. 26e), transfection efficiency of LNPs containing mCherry mRNA in pure BMDCs (FIG. 26f), transfection efficiency of LNPs containing mGFP in pure C2C12 cells (FIG. 26g), and uptake efficiency of LNPs containing Cy5-labeled mRNA in C2C12 cells (FIG. 26h). Lysotracker was used to identify the colocalization of fluorescent labeled lysosomes (Lyso-tracker) and LNPs containing Cy5-labeled mRNA in C2C12 cells in vitro (FIG. 26i). Data represent the mean±s.e.m. from a representative experiment (n=4 for (FIG. 26b-FIG. 26g) biologically independent samples) of two independent experiments. *P<0.05, ***P<0.001, ****P<0.0001. NS, not significant.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed. many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### A. Lipid Nanoparticles for Plasmid DNA Delivery to the Liver

In some embodiments, the presently disclosed subject matter provides a solid nanoparticle comprising a steroid, an ionizable cationic lipid, a helper lipid, a PEGylated lipid, and a nucleic acid payload comprising one or more nucleic acids, wherein the nanoparticle comprises: a molar ratio of the steroid to the PEGylated lipid of between about 10 and about 900; a molar ratio of the ionizable cationic lipid to the helper lipid of between about 1 and about 200; a total percentage of the ionizable lipid and the helper lipid between about 20% and about 80%; and an N to P ratio between about 2 and about 14.

In certain embodiments, the presently disclosed subject matter provides a solid nanoparticle comprising a steroid, an ionizable cationic lipid, a helper lipid, a PEGylated lipid, and a nucleic acid payload comprising one or more nucleic acids, wherein the nanoparticle comprises: a molar ratio of the steroid to the PEGylated lipid of between about 200 and about 900; a molar ratio of the ionizable cationic lipid to the helper lipid of between about 1 and about 50; a total percentage of the ionizable lipid and the helper lipid between about 35% and about 65%; and an N to P ratio between about 2 and about 14.

As used herein, the term “steroid” refers to a compound having a core structure comprising four fused rings, including three six-member cyclohexane rings (annotated as rings A, B, and C) and one five-member cyclopentane ring (annotated as the D ring) as provided in the structure immediately hereinbelow:

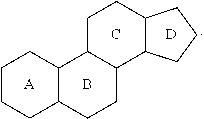

The functionality of steroids can be tuned by varying the substituent groups on the four-ring core, including, for example, one or more substituent groups selected from alkyl, alkoxyl, hydroxyl, oxo, acyl, and by the oxidation state of the rings. Steroids also can be modified by changing the ring structure, for example by cleaving one of the rings.

As used herein, the term “sterols” refers to a subgroup of steroids having a hydroxyl group at the 3-position of the A-ring. Sterols are amphipathic lipids having a polar hydroxyl group on the A ring, whereas the remainder of the aliphatic chain is non-polar. A sterol has the following general structure:

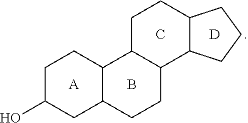

In particular embodiments, the steroid is a cholestane or cholestane derivative. In other embodiments, the steroid is a sterol or a sterol derivative. In particular embodiments, the sterol comprises cholesterol.

As used herein, the term “ionizable cationic lipid” refers to ionizable lipids that are positively charged at acidic pH to condense anionic polymers, such as nucleic acids, into lipid nanoparticles. Ionizable cationic lipids are neutral at physiological pH to minimize toxicity. Representative ionizable cationic lipids include, but are not limited to, unsaturated ionizable lipids, including DLin-MC3-DMA, OF-02, A6, and A18-Iso5-2DC18; multi-tail ionizable lipids, including 98N₁₂-5, C12-200, cKK-E12, and 9A1P9; ionizable polymeric lipids, including 7C1 and GO-C14; biodegradable ionizable lipids, including L319, 304O₁₃, OF-Deg-Lin, and 306-O12B; and branched tail ionizable lipids, including 306Oᵢ₁₀ and FTT5. Other ionizable lipids suitable for use with the presently disclosed solid nanoparticles include SM-102, ACL-0315, A9, 2,2(8,8) 4C CH3, and LP01. See, for example, Han et al., An ionizable lipid toolbox for RNA delivery, Nature Communications, 12:7233 (2021), which is incorporated herein by reference in its entirety. In particular embodiments, the ionizable cationic lipid comprises Dlin-MC3-DMA.

In some embodiments, the helper lipid is selected from a cationic lipid, a zwitterionic lipid, and an anionic lipid.

In certain embodiments, the cationic lipid is selected from N1-[2-((1S)-1-[(3-aminopropyl)amino]-4-[di(3-amino-propyl)amino]butylcarboxamido)ethyl]-3,4-di [oleyloxy]-benzamide, 1,2-di-O-octadecenyl-3-trimethylammonium propane (DOTMA), O-alkyl phosphatidylcholines, 1,2-dilauroyl-sn-glycero-3-ethylphosphocholine (12:0 EPD), 1,2-dimyristoyl-sn-glycero-3-ethylphosphocholine (14:0 EPC), 1,2-dipalmitoyl-sn-glycero-3-ethylphosphocholine (16:0 EPC), 1,2-distearoyl-sn-glycero-3-ethylphosphocholine (18:0 EPC), 1,2-dioleoyl-sn-glycero-3-ethylphosphocholine (18:1 EPC), 1-palmitoyl-2-oleoyl-sn-glycero-3-ethylphosphocholine (16:0-18:1 EPC), 1,2-dimyristoleoyl-sn-glycero-3-ethylphosphocholine (14:1 EPC), dimethyldioctadecylammonium (DDAB), N-(4-carboxybenzyl)-N,N-dimethyl-2,3-bis(oleoyloxy) propan-1-aminium (DOBAQ), 1,2-distearoyl-3-dimethylammonium-propane (18:0 DAP), 1,2-dipalmitoyl-3-dimethylammonium-propane (16:0 DAP), 1,2-dimyristoyl-3-dimethylammonium-propane (14:0 DAP), 1,2-dioleoyl-3-dimethylammonium-propane (DODAP) (18:1 DAP), 1,2-dimyristoyl-3-trimethylammonium-propane (14:0 TAP), 1,2-dipalmitoyl-3-trimethylammonium-propane (16:0 TAP), 1,2-stearoyl-3-trimethylammonium-propane (18:0 TAP), 1,2-dioleoyl-3-trimethylammonium-propane (18:1 TAP (DOTAP)), 3β-[N—(N′,N′-dimethylaminoethane)-carbamoyl]cholesterol hydrochloride (DC-Cholesterol·HCl), DC-cholesterol, N4-Cholesteryl-Spermine (GL67), 1,2-dioleyloxy-3-dimethylaminopropane (DODMA), dimyristoyltrimethylammonium propane (DMTAP), 2,3,-dioleyloxy-N-[2 (sperminecarboxamido)ethyl]-N,N-dimethyl-1-propane trifluoroacetate (DOSPA), N,N-dioleyl-N,N-dimethylammonium chloride (DODAC), 1,2-Dioleoylcarbamyl-3-Dimethylammonium-propane (DOCDAP), 1,2-Dilineoyl-3-Dimethylammonium-propane (DLINDAP), dilauryl(C_{12:0}) trimethyl ammonium propane (DLTAP), dioctadecylamidoglycyl spermine (DOGS), DC-Choi, 1,2-Dimyristyloxypropyl-3-dimethyl-hydroxyethyl ammonium bromide (DMRIE), 3-dimethylamino-2-(Cholest-5-en-3-beta-oxybutan-4-oxy)-1-(cis,cis-9,12-oc-tadecadienoxy) propane (CLinDMA), 2-[5′-(cholest-5-en-3 [beta]-oxy)-3′-oxapentoxy)-3-dimethyl-1-(ci-s,cis-9′,12′-octadecadienoxy) propane (CpLinDMA) and N,N-Dimethyl-3,4-dioleyloxybenzylamine (DMOBA), and 1,2-N,N′-Dioleylcarbamyl-3-dimethylaminopropane (DOcarbDAP), and combinations and pharmaceutically acceptable salts thereof.

In particular embodiments, the cationic lipid is selected from 1,2-dioleoyl-3-trimethylammonium-propane (DOTAP) and dimethyldioctadecyl ammonium (DDAB). In certain embodiments, the cationic lipid is 1,2-dioleoyl-3-trimethylammonium-propane (DOTAP). In certain embodiments, the cationic lipid is dimethyldioctadecyl ammonium (DDAB).

In some embodiments, the zwitterionic lipid is selected from 1,2-dioleoyl-sn-glycero-3-phosphoethanolamine (DOPE), 2-((2,3-bis(oleoyloxy) propyl)dimethylammonio) ethyl ethyl phosphate (DOCPe), and 1,2-distearoyl-sn-glycero-3-phosphocholine (DSPC), including DSPC50.

In some embodiments, the anionic lipid comprises a phospholipid. In certain embodiments, the phospholipid is selected from 1,2-dimyristoyl-sn-glycero-3-phosphate (14PA) and 1-stearoyl-2-oleoyl-sn-glycero-3-phospho-(1′-rac-glycerol) (18PG).

In certain embodiments, the solid lipid nanoparticle includes a polyethylene glycol-lipid conjugate (referred to herein as a “PEGylated lipid” or “PEG-lipid”). Representative PEGylated lipids include, but are not limited to, N-(carbonyl-methoxypolyethyleneglycolₙ)-1,2-dimyristoyl-sn-glycero-3-phosphoethanolamine (DMPE-PEGₙ where n is 350, 500, 750, 1000 or 2000), N-(carbonyl-methoxypolyethyleneglycoln)-1,2-distearoyl-sn-glycero-3-phosphoethanolamine (DSPE-PEGₙ where n is 350, 500, 750, 1000 or 2000), DSPE-polyglycelin-cyclohexyl-carboxylic acid, DSPE-polyglycelin-2-methylglutar-carboxylic acid, polyethylene glycol-dimyristolglycerol (DMG-PEG), polyethylene glycol-distearoyl glycerol (PEG-DSG), or N-octanoyl-sphingosine-1-{(succinyl [methoxy (polyethylene glycol) 2000]} (C8 PEG2000 Ceramide). In some variations of DMPE-PEGₙ where n is 350, 500, 750, 1000 or 2000, the PEG-lipid is N-(Carbonyl-methoxypolyethyleneglycol 2000)-1,2-dimyristoyl-sn-glycero-3-phosphoethanolamine (DMPE-PEG 2,000). In some variations of DSPE-PEGₙ where n is 350, 500, 750, 1000 or 2000, the PEG-lipid is N-(Carbonyl-methoxypolyethyleneglycol 2000)-1,2-distearoyl-sn-glycero-3-phosphoethanolamine (DSPE-PEG 2,000).

In certain embodiments, the PEGylated lipid comprises dimyristoyl glycerol (DMG)-polyethyleneglycol (PEG) 2000 (DMG-PEG2000).

In particular embodiments of the presently disclosed solid nanoparticle, the steroid comprises cholesterol; the ionizable cationic lipid comprises DLin-MC3-DMA; the PEGylated lipid comprises DMG-PEG2000; the nucleic acid is a mRNA; and the helper lipid is selected from 1,2-dioleoyl-3-trimethylammonium-propane (DOTAP), dimethyldioctadecyl ammonium (DDAB), 1,2-dioleoyl-sn-glycero-3-phosphoethanolamine (DOPE), DSPC, 1,2-dimyristoyl-sn-glycero-3-phosphate (14PA), and 1-stearoyl-2-oleoyl-sn-glycero-3-phospho-(1′-rac-glycerol) (18PG).

In particular embodiments of the presently disclosed solid nanoparticle, the solid nanoparticle comprises: a combined molar percentage of DLin-MC3-DMA and helper lipid ranging from about 20% to about 80%; a weight ratio of cholesterol to DMG-PEG2000 ranging from about 10 to about 500; a weight ratio of DLin-MC3-DMA to helper lipid ranging from about 1 to about 200; and a molar ratio of chargeable groups in the ionizable lipid to phosphate groups in mRNA (N/P ratio) ranging from about 4 to about 12.

In particular embodiments of the presently disclosed solid nanoparticle, the solid nanoparticle comprises: (a) about 30 molar % DOPE, about 30 molar % DLin-MC3-DMA, about 40 molar % cholesterol, about 0.40 molar % DMG-PEG2000, and a N/P ratio of about 4; (b) about 7 molar % DSPC, about 70 molar % DLin-MC3-DMA, about 20 molar % cholesterol, about 0.04 molar % DMG-PEG2000, and a N/P ratio of about 4; or (c) about 5 molar % 18PG, about 55 molar % DLin-MC3-DMA, about 40 molar % cholesterol, about 0.40 molar % DMG-PEG2000, and a N/P ratio of about 12.

Representative lipids are disclosed in U.S. Pat. No. 11,229,609 for Compositions and methods for organ specific delivery of nucleic acids, to Cheng et al., published Jan. 25, 2022, which is incorporated herein by reference in its entirety, in particular, col. 3-col. 10, and 46-52.

As used herein, the term “nucleic acid” refers to one or more of the following biomolecules, including, but small interfering ribonucleic acid (siRNA), a messenger RNA (mRNA), a micro-ribonucleic acid (miRNA), a primary micro-ribonucleic acid (pri-miRNA), a messenger ribonucleic acid (mRNA), a clustered regularly interspaced short palindromic repeats (CRISPR) related nucleic acid, a CRISPR-RNA (crRNA), a single guide ribonucleic acid (sgRNA), a trans-activating CRISPR ribonucleic acid (tracrRNA), a plasmid deoxyribonucleic acid (pDNA), a transfer ribonucleic acid (tRNA), an antisense oligonucleotide (ASO), a guide ribonucleic acid, a double stranded deoxyribonucleic acid (dsDNA), a single stranded deoxyribonucleic acid (ssDNA), a single stranded ribonucleic acid (ssRNA), a double stranded ribonucleic acid (dsRNA), a protein, a CRSIPR-associated (Cas) protein, or a combination thereof. In particular embodiments, the nucleic acid comprises plasmid DNA (pDNA) or siRNA. In certain embodiments, the nucleic acid is plasma DNA. In certain embodiments, the nucleic acid comprises siRNA. In yet more certain embodiments, the nucleic acid comprises a combination of pDNA and siRNA. In particular embodiments, the siRNA is an anti-inflammatory siRNA.

In some embodiments of the solid nanoparticle, the steroid has a molar ratio to the PEGylated lipid between about 10 and about 900, including a molar ratio of about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, and 900.

In some embodiments of the solid nanoparticle, the ionizable cationic lipid has a molar ratio to the helper lipid between about 1 to about 200, including a molar ratio of about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, and 200.

In some embodiments, the solid nanoparticle comprise a total percentage of the ionizable lipid and the helper lipid is between about 20% and about 80%, including a total percentage of about 20%, 21%, 22%, 23%, 25%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 695, 70%, 71%, 72%, 73%, 74%, 75%, 76%, 77%, 78%, 79%, and 80%.

In some embodiments, the solid nanoparticle comprises an N to P ratio between about 2 and about 14, including an N to P ratio between about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, and 14.

In some embodiments, the solid nanoparticle comprises a weight fraction of siRNA in the nucleic acid payload between about 0 to about 1, including about 0, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, and about 1. In some embodiments, the nanoparticle has a size smaller than about 400 nm, including a size of about 400 nm, 395 nm, 390 nm, 385 nm, 380 nm, 375 nm, 370 nm, 365 nm, 360 nm, 355 nm, 350 nm, 345 nm, 340 nm, 335 nm, 330 nm, 325 nm, 320 nm, 315 nm, 310 nm, 305 nm, 300 nm, 295 nm, 290 nm, 285 nm, 280 nm, 275 nm, 270 nm, 265 nm, 260 nm, 255 nm, 250 nm, 245 nm, 240 nm, 235 nm, 230 nm, 225 nm, 220 nm, 215 nm, 210 nm, 205 nm, 200 nm, 195 nm, 190 nm, 185 nm, 180 nm, 175 nm, 170 nm, 165 nm, 160 nm, 155 nm, 150 nm, 145 nm, 140 nm, 135 nm, 130 nm, 125 nm, 120 nm, 115 nm, 110 nm, 105 nm, 100 nm, 95 nm, 90 nm, 85 nm, 80 nm, 75 nm, 70 nm, 65 nm, 60 nm, 55 nm, 50 nm, 45 nm, 40 nm, 35 nm, 30 nm, 25 nm, 20 nm, 15 nm, 10 nm, and 5 nm.

### B. Methods for Delivering Lipid Nanoparticles Comprising Plasmid DNA to the Liver

In some embodiments, the presently disclosed subject matter provides a method for delivering one or more nucleic acids to a liver of a subject, the method comprising administering to a subject in need of treatment thereof a presently disclosed solid nanoparticle comprising one or more nucleic acids and described hereinabove.

In some embodiments, the one or more nucleic acids are selected from plasmid DNA (pDNA), mRNA, siRNA, and combinations thereof. In certain embodiments, the one or more nucleic acids is plasmid DNA. In certain embodiments, the one or more nucleic acids is siRNA. In particular embodiments, the one or more nucleic acids comprises a combination of plasmid DNA (pDNA) and siRNA.

In certain embodiments, the siRNA comprises an anti-inflammatory siRNA. In such embodiments, the anti-inflammatory siRNA can target a transcription factor selected from signal transducer and activator of transcription (STAT), and nuclear factor kappa-light-chain-enhancer of activated B cells (NF-κB). In particular embodiments, the method comprises reducing inflammation-induced gene silencing. In certain embodiments, an expression duration of the pDNA when co-administered with the anti-inflammatory siRNA is longer than an expression duration of the pDNA when administered alone. In certain embodiments, an expression level of the pDNA when co-administered with the anti-inflammatory siRNA substantially similar to an expression level of the pDNA when administered alone. In certain embodiments, the method comprises reducing a level within the liver of one or more of signal transducer and activator of transcription (STAT), nuclear factor kappa-light-chain-enhancer of activated B cells (NF-κB), one or more infiltrating inflammatory monocytes, and one or more apoptotic cells. In particular embodiments, the one or more infiltrating inflammatory monocytes are selected from CD45⁺ and CD11b⁺ cells.

In some embodiments, the method comprises treating one or more diseases or disorders of the liver. In particular embodiments, the one more diseases or disorders of the liver are selected from a genetic liver disease and an inflammatory liver disease. In certain embodiments, the one or more disease or disorders of the liver is selected from haemophilia B, haemophilia A, ornithine transcarbamylase (OTC) deficiency, phenylketonuria, acute intermittent porphyria, methylmalonic acidemia, familial hypercholesterolemia, Fabry, MPS type VI, Gangliosidosis GM1, Danon disease, GSD1a Von Gierke, Wilson's disease, Crigler-Najjar, primary hyperoxaluria type 1, and combinations thereof.

In particular embodiments, the method for delivering the one or more nucleic acids to a liver of a subject is selected from intravenous (i.v.) injection, oral, subcutaneous, and inhalation delivery.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

### C. Methods for Preparing Lipid Nanoparticles for Plasmid DNA Delivery to the Liver

In some embodiments, the presently disclosed subject matter provides a method for preparing a presently disclosed solid nanoparticle, the method comprising:
(a) preparing an organic phase by solubilizing a mixture of a steroid, an ionizable cationic lipid, a helper lipid, a PEGylated lipid in a polar, protic solvent at a predetermined molar ratio;
(b) preparing an aqueous phase by dissolving one or more nucleic acids in an aqueous buffer; and
(c) combining the organic phase and the aqueous phase to form the solid nanoparticle.

In some embodiments, the polar, protic solvent comprises a branched or straightchain C₁-C₄ alcohol, including a C₁, C₂, C₃, C₄ alcohol. Representative C₁-C₄ alcohols include, but are not limited to, methanol, ethanol, propanol, isopropanol, butanol, sec-butanol, isobutanol, and tert-butanol. In particular embodiments, the polar, protic solvent is ethanol.

In certain embodiments, the aqueous buffer comprises a magnesium acetate buffer.

In some embodiments, the method further comprises mixing the organic phase and the aqueous phase in a flash nanocomplexation (FNC) device. As used herein, the term “flash nanocomplexation” refers to methods that employ two or more impinging jets within a mixing chamber. As provided in International PCT Patent Application Publication No. WO2021252715 for Axisymmetric Confined Impinging Jet Mixer to Mittel et al., published Dec. 16, 2021, which is incorporated herein by reference in its entirety, these devices can include: (a) flowing a first stream comprising one or more water-soluble polycationic polymers into a confined chamber; (b) flowing a second stream comprising one or more water-soluble polyanionic polymers, e.g., plasma DNA or siRNA, into the confined chamber; and (c) impinging the first stream and the second stream in the confined chamber thereby causing the one or more water-soluble polycationic polymers and the one or more water-soluble polyanionic polymers to undergo a polyelectrolyte complexation process that continuously generates PEC nanoparticles. These types of devices that employ two or more impinging jets in a confined mixing chamber are referred to in the prior art as confined-impinging jet (CIJ) mixers. Turbulence-induced mixing can be achieved by T connectors, Tesla mixers, herringbone mixers, coaxial jet mixers, confined impinging jet mixers (CIJMs), and multi-inlet vortex mixers (MIVM). See, for example, International PCT patent application publication no. WO2020223323 for Compositionally Defined Plasmid DNA/Polycation Nanoparticles And Methods For Making The Same, to Mao et al., published Nov. 5, 2020; U.S. Patent Application Publication No. 20170042829, for Methods of Preparing Polyelectrolyte Complex Nanoparticles, to Mao et al., published Feb. 16, 2017; U.S. Pat. No. 10,441,549 for Methods of Preparing Polyelectrolyte Complex Nanoparticles, to Mao et al., issued Oct. 15, 2019, each of which is incorporated herein by reference in its entirety);

In some embodiments, the method further comprises mixing the organic phase and the aqueous phase at about a 3:1 ratio, including about a 3:1 ratio, a 2.5:1 ratio, a 2.0:1 ratio, a 1.5:1 ratio, and a 1:1 ratio.

In some embodiments, the method further comprises dialyzing the solid nanoparticle against deionized water.

D. Methods for Stimulating a Th1 and/or a Th2 Response

In some embodiments, the presently disclosed subject matter provides a method for stimulating or inducing a Type-1 T helper (Th1) and/or a Type-2 T helper (Th2) response in vivo, the method comprising administering a presently disclosed solid nanoparticle.

In certain embodiments, the steroid comprises cholesterol; the ionizable cationic lipid comprises DLin-MC3-DMA; the PEGylated lipid comprises DMG-PEG2000; the nucleic acid comprises a mRNA; and the helper lipid is selected from 1,2-dioleoyl-3-trimethylammonium-propane (DOTAP), dimethyldioctadecyl ammonium (DDAB), 1,2-dioleoyl-sn-glycero-3-phosphoethanolamine (DOPE), DSPC, 1,2-dimyristoyl-sn-glycero-3-phosphate (14PA), and 1-stearoyl-2-oleoyl-sn-glycero-3-phospho-(1′-rac-glycerol) (18PG).

In certain embodiments, the solid nanoparticle comprises: a combined molar percentage of DLin-MC3-DMA and helper lipid ranging from about 20% to about 80%; a weight ratio of cholesterol to DMG-PEG2000 ranging from about 10 to about 500; a weight ratio of DLin-MC3-DMA to helper lipid ranging from about 1 to about 200; and a molar ratio of chargeable groups in the ionizable lipid to phosphate groups in mRNA (N/P ratio) ranging from about 4 to about 12.

In particular embodiments, the solid nanoparticle comprises: (a) about 30 molar % DOPE, about 30 molar % DLin-MC3-DMA, about 40 molar % cholesterol, about 0.40 molar % DMG-PEG2000, and a N/P ratio of about 4; (b) about 7 molar % DSPC, about 70 molar % DLin-MC3-DMA, about 20 molar % cholesterol, about 0.04 molar % DMG-PEG2000, and a N/P ratio of about 4; or (c) about 5 molar % 18PG, about 55 molar % DLin-MC3-DMA, about 40 molar % cholesterol, about 0.40 molar % DMG-PEG2000, and a N/P ratio of about 12.

In certain embodiments, the method induces an immune response in Th1 only, in Th2 only, or in both Th1 and Th2.

### E. Methods for Treating a Disease or a Disorder

In other embodiments, the presently disclosed subject matter provides a method for treating a disease, disorder, or condition in subject, the method comprising administering a therapeutically effective dose of a presently disclosed solid nanoparticle to a subject in need of treatment thereof.

In certain embodiments, the disease is selected from a cancer or an infection. In particular embodiments, the cancer is selected from basal cell carcinoma, bladder cancer, breast cancer, cervical cancer, colorectal cancer, endometrial cancer, esophageal carcinoma, gastric cancer, head and neck cancer, hepatocellular carcinoma, Hodgkin's lymphoma, malignant pleural mesothelioma, Merkel cell carcinoma, metastatic melanoma, non-small cell lung cancer, renal cell carcinoma, small cell lung cancer, squamous cell carcinoma, and urothelial carcinoma.

In certain embodiments, the infection comprise a viral infection. In particular embodiments, the viral infection is selected from a coronavirus infection, a Zika virus infection, influenza, a flavivirus infection, and a human immunodeficiency virus (HIV) infection.

In other embodiments, the method further comprises administering the solid nanoparticle with one or more immune checkpoint inhibitors. In certain embodiments, the immune checkpoint inhibitor is selected from a CTLA-4 inhibitor, a PD-1 inhibitor, and a PD-L1 inhibitor. In particular embodiments, the one or more immune checkpoint inhibitors is selected from Ipilimumab, Nivolumab, Pembrolizumab, Atezolizumab, Avelumab, Durvalumab, and Cemiplimab.

The term “combination” is used in its broadest sense and means that a subject is administered at least two agents, more particularly an agent described herein and at least one other therapeutic agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

Further, the agents described herein can be administered alone or in combination with adjuvants that enhance stability of the compounds, alone or in combination with one or more therapeutic agents, facilitate administration of pharmaceutical compositions containing them in certain embodiments, provide increased dissolution or dispersion, increase inhibitory activity, provide adjunct therapy, and the like, including other active ingredients. Advantageously, such combination therapies utilize lower dosages of the conventional therapeutics, thus avoiding possible toxicity and adverse side effects incurred when those agents are used as monotherapies.

The timing of administration of an agent described herein and at least one additional therapeutic agent can be varied so long as the beneficial effects of the combination of these agents are achieved. Accordingly, the phrase “in combination with” refers to the administration of an agent described herein and at least one additional therapeutic agent either simultaneously, sequentially, or a combination thereof. Therefore, a subject administered a combination of an agent described herein and at least one additional therapeutic agent can receive one agent and at least one additional therapeutic agent at the same time (i.e., simultaneously) or at different times (i.e., sequentially, in either order, on the same day or on different days), so long as the effect of the combination of both agents is achieved in the subject.

When administered sequentially, the agents can be administered within 1, 5, 10, 30, 60, 120, 180, 240 minutes or longer of one another. In other embodiments, agents administered sequentially, can be administered within 1, 5, 10, 15, 20 or more days of one another. Where the agent described herein and at least one additional therapeutic agent are administered simultaneously, they can be administered to the subject as separate pharmaceutical compositions, each comprising either one agent or at least one additional therapeutic agent, or they can be administered to a subject as a single pharmaceutical composition comprising both agents.

When administered in combination, the effective concentration of each of the agents to elicit a particular biological response may be less than the effective concentration of each agent when administered alone, thereby allowing a reduction in the dose of one or more of the agents relative to the dose that would be needed if the agent was administered as a single agent. The effects of multiple agents may, but need not be, additive or synergistic. The agents may be administered multiple times.

In some embodiments, when administered in combination, the two or more agents can have a synergistic effect. As used herein, the terms “synergy,” “synergistic,” “synergistically” and derivations thereof, such as in a “synergistic effect” or a “synergistic combination” or a “synergistic composition” refer to circumstances under which the biological activity of a combination of an agent described herein and at least one additional therapeutic agent is greater than the sum of the biological activities of the respective agents when administered individually.

Synergy can be expressed in terms of a “Synergy Index (SI),” which generally can be determined by the method described by F. C. Kull et al., Applied Microbiology 9, 538 (1961), from the ratio determined by:

$${Q}_{a} / {Q}_{A} + {Q}_{b} / {Q}_{B} = Synergy Index ( S I )$$

wherein:
Q_{A} is the concentration of a component A, acting alone, which produced an end point in relation to component A;
Qₐ is the concentration of component A, in a mixture, which produced an end point;
Q_{B} is the concentration of a component B, acting alone, which produced an end point in relation to component B; and
Q_{b} is the concentration of component B, in a mixture, which produced an end point.

Generally, when the sum of Qₐ/Q_{A} and Q_{b}/Q_{B} is greater than one, antagonism is indicated. When the sum is equal to one, additivity is indicated. When the sum is less than one, synergism is demonstrated. The lower the SI, the greater the synergy shown by that particular mixture. Thus, a “synergistic combination” has an activity higher that what can be expected based on the observed activities of the individual components when used alone. Further, a “synergistically effective amount” of a component refers to the amount of the component necessary to elicit a synergistic effect in, for example, another therapeutic agent present in the composition.

### F. Vaccines

In other embodiments, the presently disclosed subject matter provides a vaccine comprising the solid nanoparticle disclosed herein. In certain embodiments, the vaccine is a cancer vaccine or an anti-viral vaccine.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount, or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Multi-step Screening and Compositional Optimization of Lipid Nanoparticles for Prolonged Liver-targeted DNA Delivery

### 1.1 Overview

Lipid nanoparticles (LNPs) hold great potential as an effective non-viral vector for gene therapy. Plasmid DNA (pDNA) delivery can result in extended transgene expression compared to mRNA-based technologies, yet there is a lack of systematic investigation into LNP compositions for pDNA delivery. The example provides a multi-step screening platform to identify optimized formulations for liver-targeted transgene expression. To achieve this, the role of different helper lipids and component ratios in vitro and in vivo were analyzed. Compared to mRNA LNPs, the identified formulations successfully delivered and mediated prolonged expression. By addressing different physiological barriers in a stepwise manner, this platform efficiently down selected effective candidates from a library of over 1,000 formulations. Furthermore, the expression duration was substantially extended using a pDNA/siRNA co-delivery approach that targets transcription factors regulating inflammatory response, which highlights the advantages of an extended expression profile using pDNA and offers new opportunities for pDNA-based medicine applications.

### 1.2 Scope

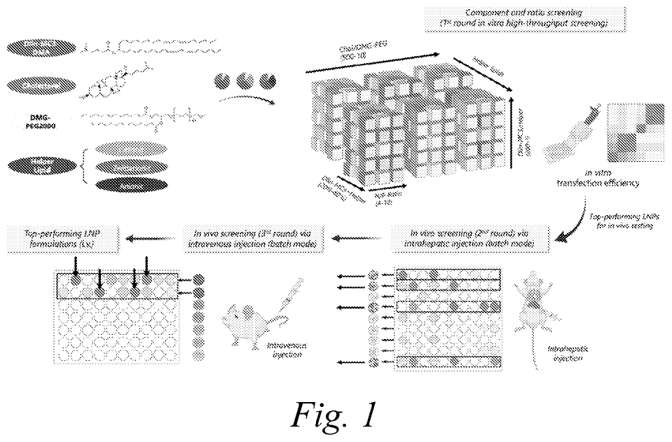

This example provides a multi-step screening platform to systematically test and analyze the liver-targeted transfection efficiency of 1,080 LNP formulations with different helper lipids and component ratios in vitro and in vivo (FIG. 1). In general, a cohort of formulations that delivered the highest levels of in vitro transfection efficiency were identified first via high-throughput screening. Inspired by the pooled diagnostic testing methods widely used during the COVID-19 pandemic, a cluster-mode screening approach was used in the initial in vivo screening step in groups of eight. These clusters were initially screened via intrahepatic injection to assess local toxicity and transgene expression levels. Clusters with minimal cytotoxicity and the highest transfection efficiencies were then selected for intravenous (i.v.) injection testing; and formulations within the clusters that demonstrated optimal liver transfection were further individually evaluated for i.v. injections. This multi-step composition screening platform was used to identify the most efficient pDNA LNP formulations from the designed library for liver-targeted transfection via i.v. administration. The transgene expression level and duration of the optimized pDNA LNPs also was compared with the widely used pDNA/PEI nanoparticles and mRNA LNPs. Hu et al., 2019. To further understand the rate-limiting steps of the in vivo gene delivery process for pDNA LNPs, the in vivo biodistribution profile, cellular uptake level, and lysosome escape capability for the top-performing formulations were examined and compared to the formulations that were less effective but shared similar characteristics.

Another challenge that must be overcome to fully realize the potential of LNP-mediated pDNA delivery is immune-mediated silencing of the transgene. Yew et al., 2002; Ballas et al., 1996; Krieg et al., 1995; Yew et al., 2000; Sparwasser et al., 1998; Hartmann and Krieg, 1999. Several approaches have been explored to extend expression duration and reduce immune response, including sequence modification to reduce CpG island density and optimization of delivery carriers. Handumrongkul et al., 2019; Yew et al., 2002.

To further improve the delivery efficiency of the top formulations, a new pDNA and siRNA co-delivery strategy is described that targets key transcription factors regulating inflammatory response pathways to reduce inflammation-induced gene silencing. Using an optimized LNP formulation, the effect of co-delivering pDNA and siRNAs against signal transducer and activator of transcription (STAT), Pfitzner et al., 2004, and nuclear factor kappa-light-chain-enhancer of activated B cells (NF-κB) Liu et al., 2017; Lawrence, 2009; Taniguchi and Karin, 2018, on the level and duration of transgene expression following i.v. administration was examined.

### 1.3 Results

1.3.1 Design of an LNP Library for the Screening of LNP-Mediated pDNA Delivery

DLin-MC3-DMA was selected as the ionizable lipid, and DMG-PEG2000 was used as the PEGylated lipid. Six helper phospholipids previously used in experimental or FDA-approved LNP formulations were chosen to represent a range of charges for testing: the cationic 1,2-dioleoyl-3-trimethylammonium-propane (DOTAP) and dimethyldioctadecyl ammonium (DDAB); the zwitterionic 1,2-dioleoyl-sn-glycero-3-phosphoethanolamine (DOPE) and DSPC; and the anionic 1,2-dimyristoyl-sn-glycero-3-phosphate (14PA) and 1-stearoyl-2-oleoyl-sn-glycero-3-phospho-(1′-rac-glycerol) (18PG). Hou et al., 2021; Cheng et al., 2020; Cheng and Lee, 2016.

Using DLin-MC3-DMA, cholesterol, DMG-PEG2000, and one of the six helper lipids, an initial library of 1,080 LNP formulations was designed by varying the following parameters: (1) ratio of DLin-MC3-DMA to helper lipid ranging from 1 to 200; (2) ratio of cholesterol to DMG-PEG2000 ranging from 10 to 500; (3) combined percentage of DLin-MC3-DMA and helper lipid ranging from 20% to 80%; and (4) N/P ratio ranging from 4 to 12. This parameter design provided a sufficiently diverse library of LNP formulations, with which the LNP-mediated pDNA delivery was programmatically tested.

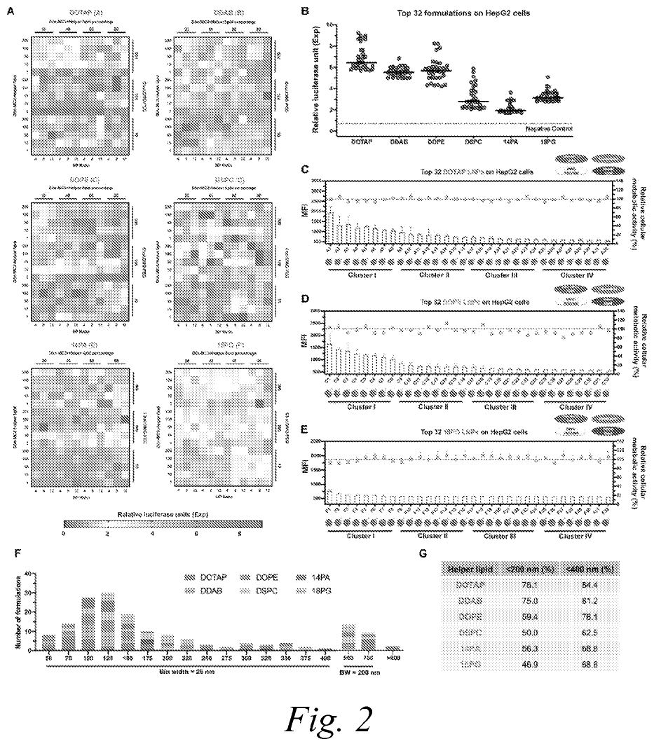

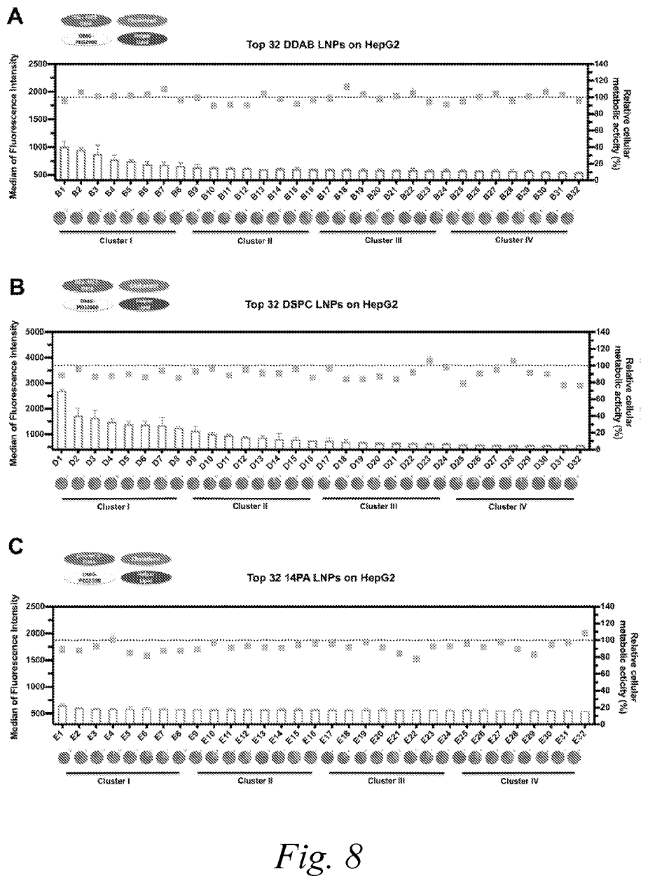

To select LNP formulations with strong liver-specific transgene expression, the pDNA delivery efficiency of the whole library was first evaluated using firefly luciferase pDNA and luciferase protein expression in HepG2 cells (a human liver cancer cell line) was measured (FIG. 2A). When fixing the helper lipid, adjusting the above-mentioned four parameters in the LNP formulations significantly varied the gene expression levels. Of the 1,080 LNP formulations, the 32 top-performing LNPs for each helper lipid group are shown in FIG. 2B and Tables 1-6. Next, the cytotoxicity was examined and the transfection efficiency of the top 32 formulations was evaluated via flow cytometry analysis. Results shown in FIG. 2C-FIG. 2E and FIG. 8 confirmed the high in vitro transfection efficiency and good biocompatibility of these LNPs.

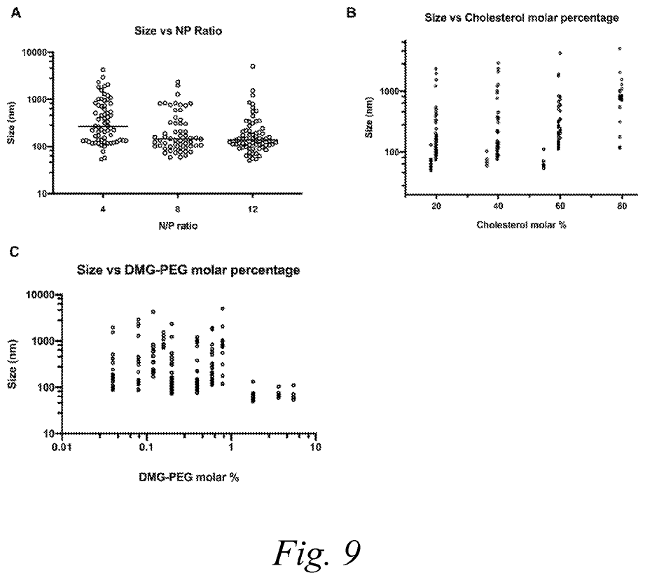

The average sizes and size distributions of top-performing LNPs was further measured using dynamic light scattering (DLS) (FIG. 2F). Results indicated most of those LNPs had a size smaller than 400 nm (approximately 73.9%), and for cationic helper lipids, the percentage of smaller LNPs (less than 400 nm) were higher (approximately 80%) compared to others (e.g., approximately 68% for anionic helper lipids) (FIG. 2G). Through detailed analysis, some correlations between the average size and formulation parameters were observed: (a) as the N/P ratio increased from 4 to 12, the average size became more uniform (i.e., with lower standard deviation) (FIG. 9A); (b) as the cholesterol content (molar percentage) increased from approximately 20% to approximately 80%, the average LNP size increased, but there was no significant change in polydispersity index (PDI) (FIG. 9B); and (c) as the DMG-PEG2000 content (molar percentage) increased from approximately 0.03% to approximately 5.5%, the LNP size became more uniform (FIG. 9C).

1.3.2 LNP-Mediated Intrahepatic pDNA Delivery Via Cluster-Mode In Vivo Screening

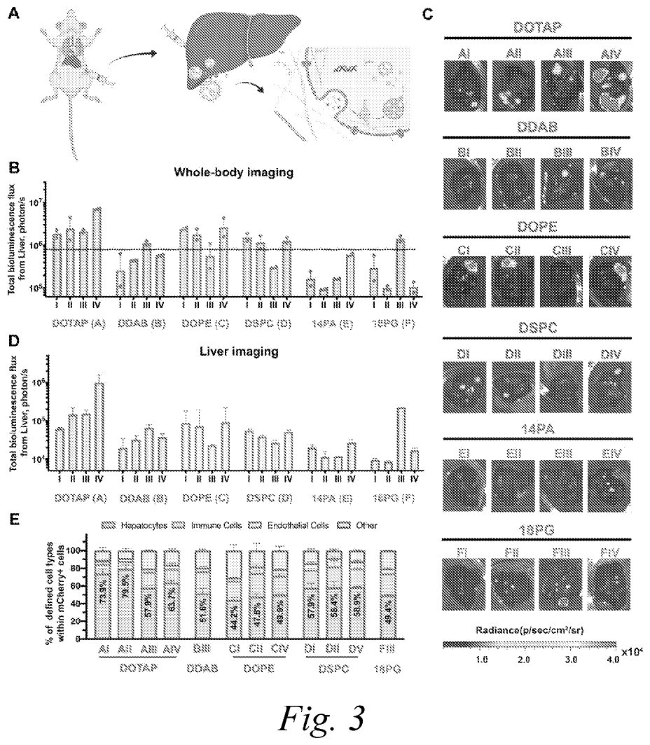

The in vivo transfection efficiency of LNPs likely differs from that in traditional in vitro assay screens due to the difference between in vivo and in vitro settings and delivery barriers. To evaluate their local in vivo tissue transfection efficiency, the LNPs that showed the highest range of in vitro transfection efficiency were tested by intrahepatic injection (FIG. 3A). As previously mentioned, a cluster mode screening method was used in this in vivo screening process, greatly reducing the number of animals, time, and cost required.

The top-performing LNP formulations were first grouped into four clusters per helper lipid (in total 24 clusters and 8 formulations per cluster) based on in vitro transfection efficiency (FIG. 1C-FIG. 1E and FIG. 8). The effects of each cluster were examined by delivering a combination of two plasmids, luciferase (Luc) (50%) and mCherry (50%) pDNA, at a total dose of 3 μg pDNA per mouse via intrahepatic injection. Unexpectedly, clusters with high transfection efficiency in vitro were not necessarily the top-performing clusters in vivo; and this finding applied both among clusters that include the same helper lipid and among all 24 clusters (FIG. 3B-FIG. 3D). For example, cluster AIV, which contained the eight DOTAP formulations with the lowest in vitro transfection efficiency in the DOTAP group, produced an average bioluminescence signal (Luc expression) 17.2 times higher than that of cluster AI (composed of the eight top-performing DOTAP formulations in vitro) (FIG. 3D). Moreover, clusters that showed a moderate efficiency in vitro could have potent transfection efficiency in vivo. For instance, cluster FIII had a surprisingly high transfection efficiency in contrast to its in vitro performance. In addition, the charge of the helper lipids significantly influenced the transfection efficiency; cationic lipids like DOTAP had a more potent effect than others, especially compared to the least effective anionic lipid, 14PA. However, although DOTAP and DDAB are both cationic lipids, the local transfection of DDAB clusters (such as BI, BII, and BIV) in the liver was low. Similarly, most clusters composed of anionic helper lipids had limited local transfection in the liver, but there was a unique cluster (FIII) in the 18PG group that achieved relatively high transfection efficiency. Likewise, although DOPE and DSPC clusters had high transfection efficiency overall, clusters CIII and DIII showed lower efficiency. No significant luciferase expression was detected in other organs based on the whole-body imaging analysis.

To gain an initial estimate of the cell types transfected by the 12 top-performing clusters (shown in FIG. 3B above the dashed line), mCherry expression levels in various cell types within the liver were quantified using flow cytometry (FIG. 3E). After a single intrahepatic injection, in all top 12 clusters, around 50% of the transfected cells in the liver were hepatocytes followed by the second majority of immune cells. Particularly, for cluster AI and AII, the transfection efficiency in hepatocytes reached as high as 73.9% and 79.5%, respectively. This result showed that these clusters contained LNP formulations that could have potent liver-specific transfection. Based on the IVIS results (FIG. 3B and FIG. 3D), the top 12 clusters of LNPs (96 formulations in total) were successfully as candidate clusters for further evaluation of their stability within blood circulation, and tissue-specific transfection efficiency following systemic delivery.

1.3.3 LNP-Mediated, Liver-Specific pDNA Delivery Via Cluster-Mode Testing

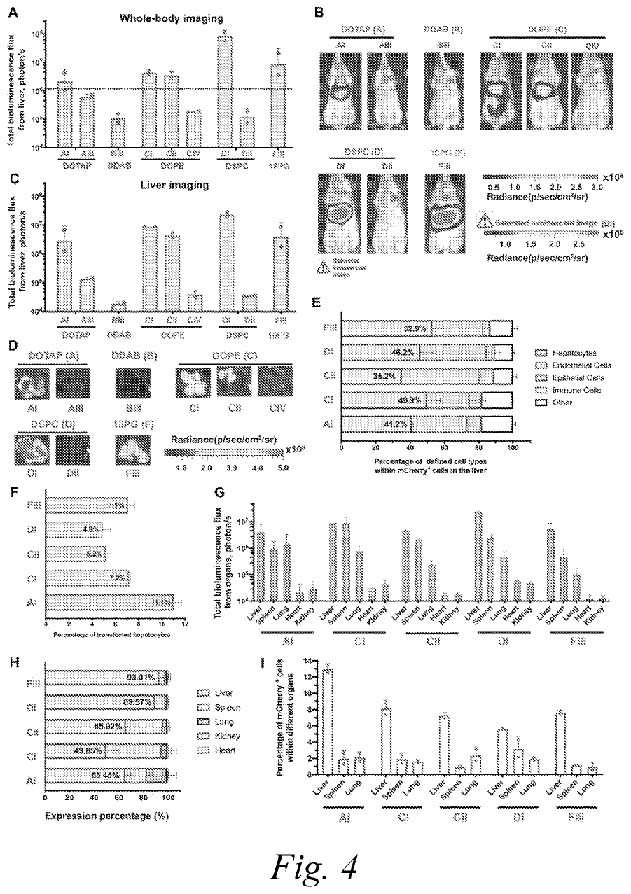

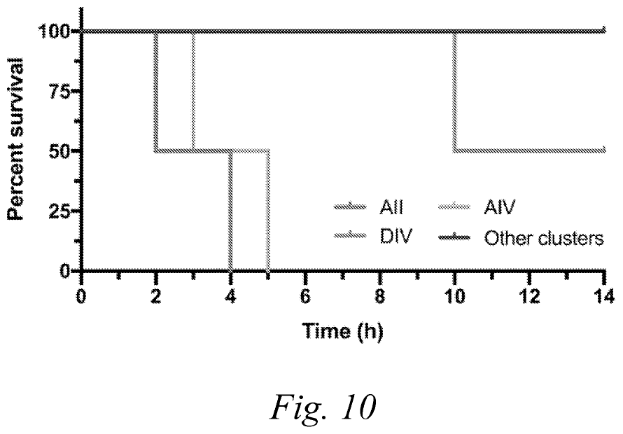

The 12 clusters that demonstrated the highest transgene expression levels in the liver were then examined for performance via the i.v route. The same payload, luciferase (Luc) (50%) and mCherry (50%) pDNA, were encapsulated in these LNPs, which were administering i.v at a total pDNA dose of 100 μg per mouse. Three clusters (AII, AIV, and DIV) among the 12 tested showed significant toxicity after i.v administration and were excluded from further evaluation (FIG. 10). Five clusters (AI, CI, CII, DI and FIII) were the most efficient clusters for liver-specific transgene expression (FIG. 4A-FIG. 4D). Compared with other clusters, these five clusters gave an average of 2 to 3 orders of magnitude higher luciferase expression in the liver at 12 h after i.v. administration measured by IVIS imaging. For example, cluster DI was 660 times higher than that of cluster DII. For these top five clusters of LNPs, delivery to specific cell types within the liver was further quantified using flow cytometry to detect mCherry expression, which revealed that about 40% of transfected cells in the liver were hepatocytes and about 7% of the total hepatocytes in the liver were successfully transfected (FIG. 4E and FIG. 4F).

Next, the transgene expression level of the top five clusters was evaluated in other organs including the spleen, lung, kidney, and heart (FIG. 4G). Based on the relative Luc expression in each organ, two clusters, DI and FIII, yielded high liver-specific transfection efficiency; 89.6% of bioluminescence among the organs was from the liver for DI, and 93.0% for FIII (FIG. 4H). In addition, higher levels of transgene expression in the spleen were observed for clusters CI (45.2%) and CII (30.0%). The percentage of transfected cells in three major organs (liver, spleen, and lung) were further evaluated through flow cytometry; for all five clusters, roughly 10% of cells in the liver were transfected based on mCherry expression (FIG. 4I). Based on these data, clusters DI and FIII were selected for further characterization.

1.3.4 Formulations for Liver-Specific pDNA Delivery

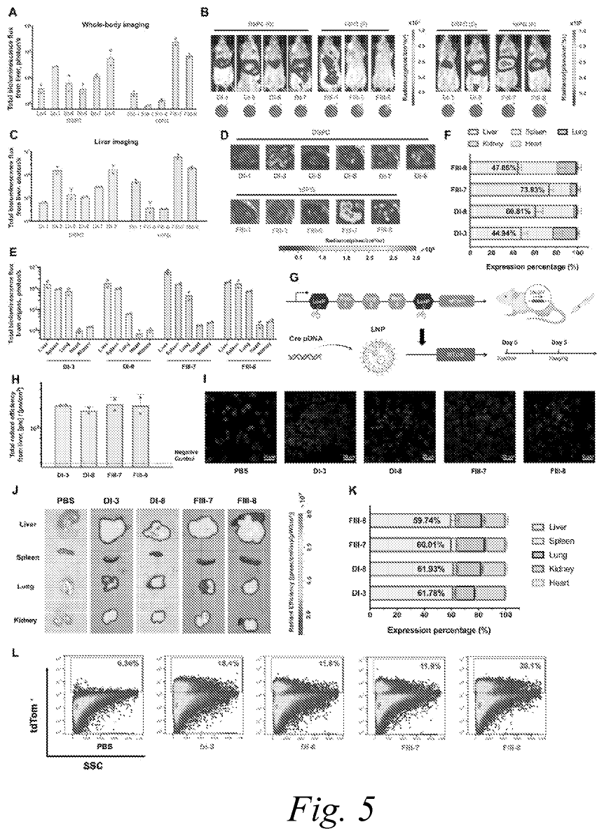

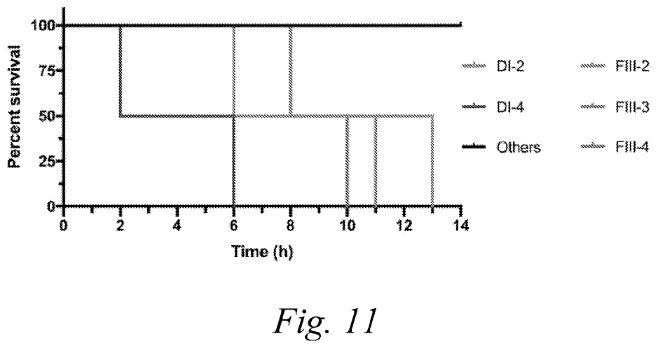

The transfection efficiencies of the 16 individual LNP formulations within the DI and FIII clusters were further examined following i.v. injection at a total pDNA dose of 50 μg per mouse using the same Luc/mCherry combination (50/50) payload. Five of the 16 formulations (DI-2, DI-4, FIII-2, FIII-3 and FIII-4) showed high toxicity following i.v. administration and were excluded from further evaluation (FIG. 11). Based on IVIS results shown in FIG. 5A-FIG. 5D, four individual formulations (DI-3, DI-8, FIII-7, and FIII-8) showed the highest levels of Luc expression in the liver. The best-performing formulation, FIII-7, demonstrated a 300-fold higher Luc expression than FIII-5, another formulation within the same cluster. The transgene expression levels in other major organs mediated by the top four formulations also were evaluated (FIG. 5E). Of the total bioluminescence among various organs, 73.9% occurred in the liver for FIII-7 and 60.8% for DI-8, with both formulations showing exclusive liver-specific transgene expression (FIG. 5F). These top four formulations (DI-3, DI-8, FIII-7, and FIII-8) were therefore advanced to further testing using an orthogonal assay to measure liver-specific transgene expression.

1.3.5. Validation of Top Formulations for Liver-Specific pDNA Delivery

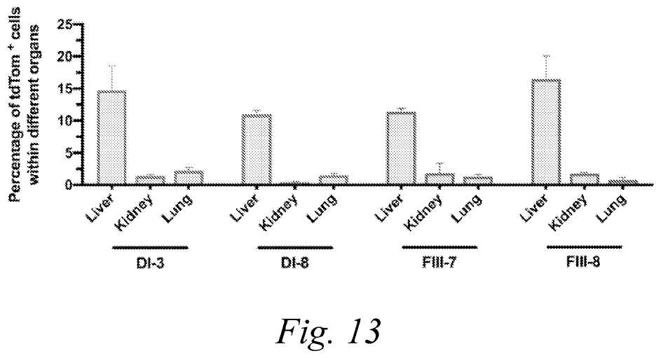

To further validate the efficiencies of the top four LNP formulations in mediating liver-specific gene delivery, genetically engineered tdTom reporter mice (Ai9 mice) containing a LoxP-flanked stop cassette that prevents expression of the tdTom protein were utilized. This mouse model allows detection of the gene-edited cells as a result of Cre expression (FIG. 5G). When Cre recombinase is introduced into the reporter mouse cells, it recombines the DNA at the LoxP sites to excise the stop cassette, which permits the expression of fluorescent tdTom. The four formulations (DI-3, DI-8, FIII-7, and FIII-8) were used to deliver Cre recombinase pDNA following i.v. injection (25 μg pDNA/mouse). Three days post injection, a high tdTom signal was detected in the liver (FIG. 5H); and tdTom-positive cells were easily observed using confocal imaging of tissue sections (FIG. 5I). Fluorescent signal also was observed in other organs (FIG. 5J, FIG. S5), but about 60% of the total tdTom expression among imaged organs was from the liver for all four LNPs (FIG. 5K). Flow cytometry was used to further quantify the percentage of gene-edited cells in the liver and found that about 20% of the cells were successfully edited by treatment with FIII-8 (FIG. 5L, FIG. 13). Based on high transfection efficiency and high biocompatibility, the top four LNPs may be applicable to liver-targeting gene therapy via systemic delivery.

### 1.3.6 Biodistribution, Cellular Uptake and Endosomal Escape Level of Top-Performing Formulations

Having identified the top four LNP formulations using the multi-step screening platform, the mechanism remains unclear. Without wishing to bound to any one particular theory, it is thought that: enhanced liver-targeting transfection is the result of (1) the tissue-specific biodistribution of LNPs, (2) the differential cellular uptake profiles of LNPs following distribution into the local tissue, and (3) the differential endosomal escape or DNA release abilities of LNPs, even between formulations with similar biodistribution and cellular uptake levels.

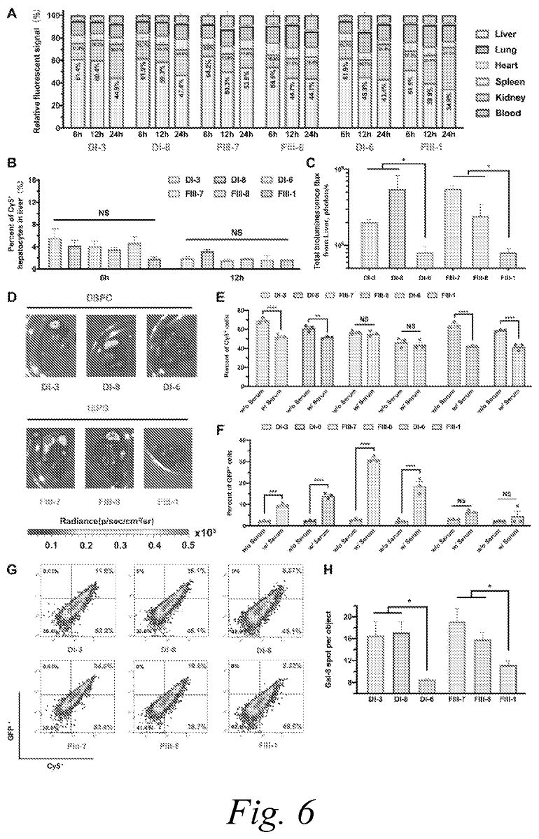

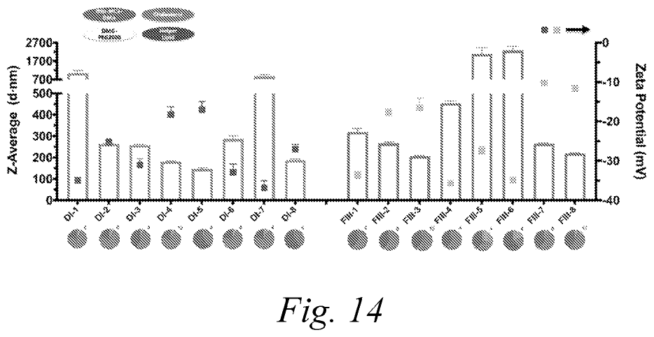

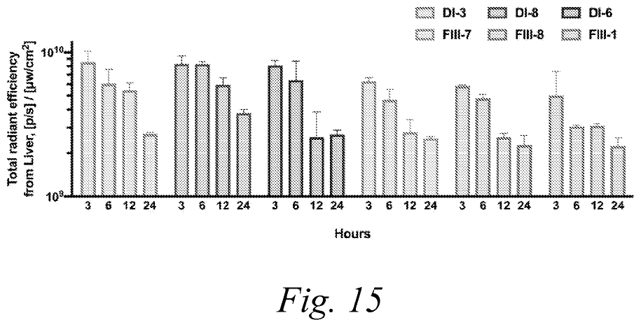

To test these hypotheses, the biodistribution of the four top-performing LNPs was evaluated at 6 h, 12 h, and 24 h after i.v. injection using Cy5-labeled pDNA. For comparison, two LNP formulations (DI-6 and FIII-1) that were less effective, but possessed similar characteristics (size and zeta potential) to the top-performing ones were included (FIG. 14). IVIS imaging showed that all six selected formulations, independent of transfection performance, had similar biodistribution profiles at all time points (FIG. 6A, FIG. 15). In all cases approximately 60% of LNPs were distributed to the liver at 6 h after i.v. administration. In addition, the similar uptake levels by hepatocytes were observed for all six formulations, with flow cytometry assessment indicating that the high transfection by the top formulations was not caused by differences in biodistribution nor cellular uptake level (FIG. 6B).

To verify that the transfection efficiency of LNPs was not related to biodistribution, the transfection efficiency of 6 LNPs was checked by administering the same dose of the 6 LNPs via intrahepatic injection. The results in FIG. 6C and FIG. 6D showed that although the same dosage was delivered to the liver, the local transfection efficiency was significantly different. Compared with DI-6 and FIII-1, the top four formulations indeed provided a higher transfection efficiency. Thus, regardless of the delivery route, transfection efficiency was not strictly related to biodistribution.

To verify that endosomal escape and/or DNA release improved the transfection efficiency of LNP formulations, primary mouse hepatocytes were isolated and transfected with the six LNPs (DI-3, DI-8, FIII-7, FIII-8, DI-6, and FIII-1). To simulate the situation in vivo, LNPs were incubated with fresh mouse serum at a 2:1 volume ratio (LNP/serum) at 37° C. for 30 min before dosing to cells. With or without pre-incubation with mouse serum, all six LNPs exhibited similar uptake levels, which is consistent with the in vivo cellular uptake level observed (FIG. 6E). While pre-incubation with mouse serum did not significantly impact cellular uptake level, the transfection efficiency of the best four LNPs was greatly improved; the percentage of GFP positive cells increased by 12 times after serum pre-incubation for FIII-7 (FIG. 6F and FIG. 6G), whereas no significant difference was observed in the transfection efficiencies of DI-6 and FIII-1 compared to the drastic increases for the top four formulations. Furthermore, the endosomal escape capability of selected formulations was examined by quantitative Cellomics high-content analysis on an established B16-Gal8-GFP cell line. Karlsson et al., 2020; Hu et al., 2021. The results showed in FIG. 6H indicated that the top four formulations (DI-3, DI-8, FIII-7, and FIII-8) have a relatively higher endosomal escape capacity.

The above results showed that LNPs with similar size and zeta potential yielded a similar biodistribution and cellular uptake profile after i.v. injection; the varied endosomal escape capability of different LNP formulations was likely the determining factor for difference in the transfection efficiency. Moreover, the significant difference observed between groups incubated with or without serum suggested that the discrepancies between in vivo and in vitro experiments may be closely related to serum-mediated opsonization of the LNPs.

1.3.7 Extending Transgene Expression Duration of LNPs by Co-Delivery with Anti-Inflammatory siRNAs

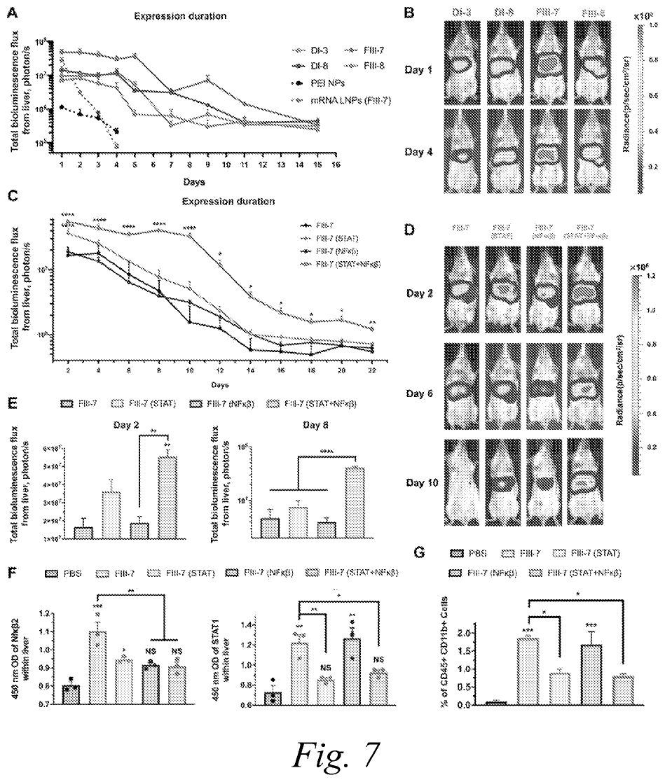

The duration of expression within the liver in BALB/c mice was monitored following the i.v. injection of the top four formulations (DI-3, DI-8, FIII-7, and FIII-8) (FIG. 7A and FIG. 7B). The initial expression levels of the four formulations were consistent with data shown above, and the expression was maintained at a similar level for about 4 days before declined over 3 to 7 days. A control group loaded with Luc mRNA using FIII-7 LNP formulation also was tested (5 μg mRNA per mice, i.v.). Although the initial expression within the liver by mRNA LNPs was comparable on day 1 to that mediated by pDNA LNPs (25 μg pDNA/mouse), the expression level dropped by 10-fold on day 2 and decreased by more than 300-fold on day 4. A polycationic carrier Polyplus in vivo-jet PEI used to generate pDNA/PEI nanoparticles (PEI NPs) also was tested for comparison. The majority of Luc expression level mediated by PEI NPs were found in the lung, and transgene expression in the liver was much lower than FIII-7 pDNA LNPs (approximately 2.4%) on day 1. Hu et al., 2019. Thus, pDNA LNPs provided substantially longer transgene expression than either of the tested comparators.

To further extend the expression of pDNA, several methods are being explored to reduce undesirable innate immune activation and host toxicity, such as sequence modification (CpG-depleted pDNA) and delivery vector optimization. Handumrongkul et al., 2019; Yew et al., 2002. Here, the effect of co-delivery of pDNA and anti-inflammatory siRNA (against signal transducer and activator of transcription (STAT) and nuclear factor kappa-light-chain-enhancer of activated B cells (NF-κB)) with pDNA to prolong the duration of expression was tested. Pfitzner et al., 2004; Liu et al., 2017; Taniguchi and Karin, 2018; Salas et al., 2020; Jay and Eric, 2009.

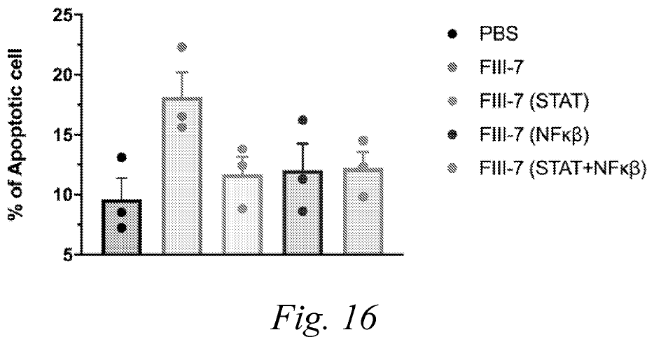

For these experiments, 2.5 μg siRNA/mouse was used for each transcription factor. As FIG. 7C-FIG. 7E showed, the expression duration mediated by FIII-7 LNPs was extended from 5 days to 10 days without a significant change in expression level when STAT and NF-κB were co-delivered. In addition, elevated initial expression levels within the liver received FIII-7 (STAT+NF-κB) LNPs and FIII-7 (STAT) LNPs were 3.4- and 2.2-fold higher on day 2, respectively, than FIII-7 LNPs alone (FIG. 7E). At 7 days after administration, the STAT1 and NF-κB2 level within the liver was examined by ELISA. As FIG. 7F shows, the elevated levels of both inflammatory transcription factors were observed after i.v. injection of FIII-7 LNPs; and coupling with anti-inflammatory siRNAs, the transcription factor levels were significantly reduced. For the FIII-7 (STAT+NF-κβ) LNP group, compared with the FIII-7 LNPs, the levels of NF-κβ2 and STAT1 were reduced by approximately 17.6% and approximately 24.5%, respectively. In addition, lower levels of infiltrating inflammatory monocytes (CD45⁺CD11b⁺ cells) and apoptotic cells within the liver also were observed (FIG. 7G, FIG. 16). Zigmond et al., 2014. These combined effects were therefore sufficient to substantially increase the level the transgene expression and extend the duration.

### 1.4 Discussion

The translation of LNPs as a carrier for gene delivery has progressed tremendously over the past couple of years due to the success of COVID-19 mRNA vaccines. The biosafety and translatability of the LNPs have been demonstrated, making it extremely attractive for the field of gene therapy. Extending this to other therapeutic areas, however, requires systematic screening and optimization of the LNP formulation based on the requirements for specific target cell and tissue types, expression duration, and the like. Both the choice of components and their molar ratios can drastically influence the efficiency of nucleic acid encapsulation efficiency, stability of LNPs, cellular uptake, endosomal escape, and the release profile of the payload. Zhang et al., 2021; Wang et al., 2015.

More importantly, screening for in vivo targets represents a greater challenge due to the throughput limitation. In developing this multi-step composition screening process, the top performing LNP formulations from the designed library consisting of 1,080 formulations for liver-targeted transfection through i.v. administration were successfully identified. This designed screening process considers both in vitro and in vivo steps, which allows for rapid identification of effective formulations through a programmatic approach, and drastically reduce animal usage. This process can easily be translated to the development of delivery systems for other nucleic acid payloads including, but not limited to, mRNA, siRNA, and miRNA, as well as alternative administrative routes, such as oral, subcutaneous, and inhalation delivery.

Previous reports on LNP-enabled gene delivery systems argue that LNP composition influences tissue-targeting and transfection. Cheng et al., 2020; Zhang et al., 2021; Wang et al., 2015; Cheng and Lee, 2016. The presently disclosed subject matter revealed that the preferentially high transfection efficiency in the liver mediated by these selected LNP formulations is not directly related to in vivo biodistribution nor cellular uptake efficiency. Rather the intracellular trafficking steps including endolysosomal escape and pDNA release play a more critical role. It is entirely possible for LNPs with similar characteristics and similar distribution among different organs and tissues, to give different tissue-specific transfection outcomes and/or yield different transfection levels across different cell types. This outcome is most clearly seen in the direct comparisons between top-performing formulations (DI-3, DI-8, FIII-7 and FIII-8) and selected formulations with less efficacy, but similar characteristics (DI-6 and FIII-1). These findings further highlight the need for a rationale, multi-step screening approach when evaluating LNP formulations for each specific target organ and gene medicine application.

For therapeutic gene delivery, pDNA as a therapeutic payload offers unique advantages including more persistent transgene expression, higher stability, and a lower production cost, compared with the mRNA cargo. This result also showed that optimized pDNA LNPs yielded 4 to 5-day sustained transgene expression as opposed to sharp drop over two days. The innate immune activation against pDNA has been reported to induce gene silencing and inflammation response. Previously, CpG-depletion in pDNA sequence has been explored to address this issue. Here, a new approach via co-delivery of anti-inflammatory siRNAs with pDNA in the same LNP formulation that can effectively extend the transgene expression without relying on pDNA sequence modification is demonstrated. The inclusion of anti-inflammatory siRNAs reduced the recruitment of immune cells and the number of apoptotic cells after treatment with LNPs. Even with moderate reduction of STAT and NF-κB levels, this approach yields substantial improvement in the overall level and duration of the transgene in the liver. This strategy requires no sequence modification or complex delivery vehicles and can be easily adopted for other delivery systems and applications.

### 1.5 Summary

Overall, the presently disclosed subject matter provides a multi-step composition screening platform that allowed the best-performing pDNA LNPs for liver-specific transgene expression to be rapidly and programmatically identified from an LNP library of over 1,000 formulations. This platform combines in vitro and in vivo screening strategies; it can be extended to other carrier systems and potentially for various administration routes. In addition, it was revealed that the preferential transfection in the liver vs. other organs/tissues of the selected LNPs is not directly related to targeted in vivo distribution or cellular uptake efficiency of LNPs; rather the intracellular trafficking events including lysosome escape, DNA release, and the like play a more critical role. It can be deduced that LNPs with similar physical characteristics are distributed among different organs in a similar manner; but they show tissue-specific differences in transfection across different cell types due to differences in intracellular cellular trafficking efficiency in a composition-dependent manner. Finally, an innovative strategy that co-delivers anti-inflammatory siRNA and pDNA to further extend the expression of pDNA therapy was developed. This LNP-based co-delivery strategy further highlights the unique advantages of an extended transgene expression profile using pDNA delivery and offers new opportunities for pDNA-based gene medicine applications.

### 1.6 Materials and Methods

### 1.6.1 Materials

DLin-MC3-DMA was purchased from MedKoo Biosciences. DSPC, DOPE, DOTAP, DDAB, 18PG (sodium salt) and 14PA (sodium salt) were purchased from Avanti Polar Lipids. Cholesterol was purchased from Sigma. DMG-PEG (MW 2000) (DMG-PEG2000) was purchased from NOF America Corporation. Reporter lysis buffer and luciferin assay solution were purchased from Promega. All pDNA was purchased from Aldevron. D-Luciferin (sodium salt) was purchased from Gold Biotechnology.

### 1.6.2 Cell Culture and High-Throughput Screening for Transfection Studies

For monolayer culture studies, HepG2 cells (American Type Culture Collection, USA) were seeded into 96-well plates at a cell density of 10,000 cells per well one day prior to transfection. The particles prepared were pipetted into EMEM medium at a final particle concentration of 1 μg pDNA mL⁻¹. For example, 8 μL of a particle suspension at 25 μg pDNA mL⁻¹ was pipetted into the 200 μL culture media in the wells. A 24-h incubation was followed to allow transgene expression. When characterizing luciferase as the reporter, cells were lysed by reporter lysis buffer (Promega) using two freeze-thaw cycles, with the lysate characterized by a luminometer upon addition of luciferin assay solution (Promega) against a standard curve generated using luciferase samples (Promega).

### 1.6.3 LNP Synthesis and Characterization

An organic phase was prepared by solubilizing with ethanol a mixture of the helper lipid (DOTAP, DDAB, DOPE, DSPC, 14PA, 18PG) (Avanti), cholesterol (Sigma-Aldrich), DMG-PEG2000 (Avanti) and Dlin-MC3 DMA at a predetermined molar ratio. The aqueous phase was prepared in 25 mM magnesium acetate buffer (pH 4.0, Fisher) with Luc pDNA (firefly mLuc, Translate), mCherry pDNA, Cre pDNA or Cy5-labeled pDNA. All pDNAs were stored at −20° C. and were allowed to thaw on ice before use. For high-throughput screening, LNPs were prepared in a 96-well plate or 1.5 mL microcentrifuge tubes by directly adding ethanol phase to aqueous phase. For in vitro screening, LNPs were directly incubated with cells without dialysis. For in vivo batch analysis screening, LNPs in each cluster were mixed and dialyzed against DI water before injection into mice. For larger scale LNP production, the ethanol and aqueous phases were mixed at a 3:1 ratio in a FNC device using syringe pumps as previously described. Resultant LNPs were dialyzed against DI water in a 100,000 MWCO cassette (Fisher) at 4° C. for 24 h and were stored at 4° C. before injection. For cellular uptake studies in vitro, the LNPs were incubated with serum with volume ratio 2:1 (LNP/serum) for 30 min in 37° C. The size, polydispersity index and zeta potentials of LNPs were measured using dynamic light scattering (ZetaPALS, Brookhaven Instruments). Diameters are reported as the intensity mean average.

### 1.6.4 Animals and Primary Cells

All animal procedures were performed with ethical compliance and approval by the Johns Hopkins Institutional Animal Care and Use Committee. Female BALB/c mice (6-8 weeks) were obtained from the Jackson Laboratory and Ai9 mice bred in Johns Hopkins Animal Facilities and randomly grouped. The LNPs were injected i.v. via mouse lateral tail vein or intrahepatically via a small incision under sternum at a predetermined dose per mouse. The LNP suspensions were concentrated to 200 μg mL⁻¹ of pDNA by an Amicon Ultra-2 centrifugal filter unit with a MWCO of 100 kDa. The mice were injected intraperitoneally with 100 μL of 30 mg mL⁻¹ D-luciferin solution and were anesthetized in a ventilated anesthesia chamber with 1.5% isoflurane in oxygen and imaged at 5 min after the injection with an in vivo imaging system (IVIS, PerkinElmer). Luminescence was quantified using the Living Image software (PerkinElmer). For experiments in Ai9 mice, the Cre pDNA LNP formulations were prepared as described above and administered via i.v. injections at a pDNA dose of 25 μg per mouse (n=2). After three days, mice were sacrificed, and the major organs were imaged using IVIS. For in vitro transfection in primary hepatocytes, cells were isolated by using Hepatocyte Isolation System (Tissue Dissociation/Cell Isolation), BioAssay™ Kit (Cat. H2006-02) following manufacturer's protocols. The hepatocytes were cultured in RPMI1640 medium supplemented with 10% fetal bovine serum at 37° C. in 5% CO₂.

### 1.6.5 Cell Isolation and Staining for Flow Cytometry

To quantify the mCherry+ or tdTom+ cells among different cell types in each organ, cell isolation and staining was performed, followed by flow cytometry analysis. For hepatocyte isolation, a two-step collagenase perfusion was executed as described previously. Briefly, mice were anesthetized using isoflurane then fixed. Perfusion, initially using liver perfusion medium (Thermo Fisher) for 7-10 min, then switching to liver digestion medium (Thermo Fisher) for another 7-10 min, was performed. The liver was collected on a plate containing 10 mL of liver digestion medium and cut to release the hepatocytes. The released hepatocytes were then collected and washed with ice-cold hepatocyte wash medium (Thermo Fisher) and centrifuged at 50×g for 5 min. The supernatant was decanted, and the pellet was resuspended with an ice-cold hepatocyte wash medium. The cell suspension was passed through a 100-μm filter. The hepatocyte suspension was washed twice with ice-cold hepatocyte wash medium and once with PBS via centrifugation (50×g) for 5 min. Afterwards, the hepatocytes were further strained through a 100-μm filter and centrifuged at 50×g for 5 min, and cells were resuspended in 500 μL of staining buffer. The antibodies used here were Brilliant Violet 605 anti-mouse CD45, Cyanine 5 anti-mouse CD326, Alexa Fluor 488 anti-mouse CD31, PerCP-Cyanine 5.5 anti-mouse CD11b (BioLegend), and APC anti-mouse CD11c. Flow data were acquired on SH800 and analyzed using FlowJo software.

For isolation and staining of spleen cells, the removed spleen was minced using a sterile blade and homogenized in 250 μL of digestion medium (45 units μL⁻¹ collagenase I, 25 units μL⁻¹ DNase I and 30 units μL⁻¹ hyaluronidase). The spleen solution was transferred into a 15-mL tube that contained 5-10 mL of digestion medium. Next, the spleen solution was filtered using a 70-μm filter and washed once with PBS. Cells was pelleted at 300×g for 5 min at 4° C., and resuspended in 2 mL of red blood cell lysis buffer (BioLegend) and incubated on ice for 5 min. After incubation, 4 mL of cell staining buffer (BioLegend) was added and centrifuged again at 300×g for 5 min. Cell pellet was washed with staining buffer for 3 times and stained with antibodies (total volume 100 μL) for 20 min in the dark at 4° C. The stained cells were washed twice with 1 mL of PBS, then resuspended in 500 μL PBS for flow cytometry analysis. The antibodies used include Brilliant Violet 605 anti-mouse CD45 (BioLegend), PerCP-Cyanine 5.5 anti-mouse CD11b (BioLegend), APC anti-mouse CD11c, FITC anti-mouse CD3 and PE-Cyanine 7 anti-mouse CD19 (BioLegend).

For isolation and staining of lung cells, isolated lungs were minced using a sterile blade and then transferred into a 15-mL tube that contained 10 mL of 2× digestion medium (90 units μL⁻¹ collagenase I, 50 units μL⁻¹ DNase I, and 60 units μL⁻¹ hyaluronidase) and incubated at 37° C. for 1 h with shaking. After incubation, any remaining lung tissue was homogenized. The following steps were similar to the spleen protocol described above. The antibodies used here were the same to that of hepatocytes.

### 1.6.6 Quantitative Endosomal Escape Assessments by Cellomics Analysis

B16F10 cells expressing GFP-coupled galectin-8 (GFP-Gal8) was obtained by transfection using plasmids encoding Piggybac-transferase (Hera BioLabs) and Piggybac-transposon-GFP-Gal8 (Addgene) and a poly(β-amino ester) (PBAE) carrier, Karlsson et al., 2020, then sorted by an SH800 cell sorter (Sony) twice. The cells were cultured in DMEM supplemented with 10% FBS at 100,000 cells per well. The particles were dosed at 24 h later as described above. After incubation for predetermined times, cells were washed by PBS for three times, fixed by 4% paraformaldehyde (PFA) solution, stained by Hoechst 33342, and then washed by PBS for three times. The plates were analyzed by a CellInsight CX7 High-content Analysis (HCA) platform (Thermo Fisher Scientific). The analysis was conducted according to a previously reported method. Hu et al., 2019.

Briefly, imaging was conducted at 20× magnification with a resolution of 1104×1104 pixel² per field correlating with an area of 501.2×501.2 μm². A total of 30 fields were analyzed inside each well of the plates; and the well-averaged results were generated by averaging all the cells in all the fields in each well.

### 1.6.7 Statistical Analysis

A two-tailed Student's t-test or a one-way analysis of variance (ANOVA) was performed when comparing two groups or more than two groups, respectively. Statistical analysis was performed using Microsoft Excel and Prism 7.0 (GraphPad). A difference is considered significant if P<0.05 (*P<0.05, **P<0.01, ***P<0.001, ****P<0.0001).

TABLE 1
Formulation details for Top 32 LNPs in
Group A with DOTAP as the helper lipid

| | Molar % | | | | |
|---|---|---|---|---|---|
| Code | DOTAP | Dlin-MC3 | Chol | DMG-PEG | N/P Ratio |
| A1 | 5.45 | 54.55 | 39.60 | 0.40 | 4 |
| A2 | 5.45 | 54.55 | 39.60 | 0.40 | 12 |
| A3 | 20.00 | 20.00 | 59.88 | 0.12 | 8 |
| A4 | 5.45 | 54.55 | 39.60 | 0.40 | 8 |
| A5 | 40.00 | 40.00 | 19.96 | 0.04 | 12 |
| A6 | 3.64 | 36.36 | 59.41 | 0.59 | 12 |
| A7 | 30.00 | 30.00 | 39.92 | 0.08 | 8 |
| A8 | 3.64 | 36.36 | 59.41 | 0.59 | 8 |
| A9 | 3.64 | 36.36 | 59.41 | 0.59 | 4 |
| A10 | 10.00 | 10.00 | 79.20 | 0.80 | 8 |
| A11 | 20.00 | 20.00 | 59.41 | 0.59 | 12 |
| A12 | 30.00 | 30.00 | 39.60 | 0.40 | 8 |
| A13 | 30.00 | 30.00 | 39.92 | 0.08 | 12 |
| A14 | 40.00 | 40.00 | 19.80 | 0.20 | 8 |
| A15 | 40.00 | 40.00 | 19.80 | 0.20 | 12 |
| A16 | 30.00 | 30.00 | 39.60 | 0.40 | 12 |
| A17 | 10.00 | 10.00 | 79.21 | 0.79 | 12 |
| A18 | 20.00 | 20.00 | 59.88 | 0.12 | 4 |
| A19 | 20.00 | 20.00 | 54.55 | 5.45 | 8 |
| A20 | 40.00 | 40.00 | 19.80 | 0.20 | 4 |
| A21 | 20.00 | 20.00 | 59.41 | 0.59 | 8 |
| A22 | 10.00 | 10.00 | 79.84 | 0.16 | 4 |
| A23 | 1.82 | 18.18 | 79.21 | 0.79 | 12 |
| A24 | 20.00 | 20.00 | 54.55 | 5.45 | 4 |
| A25 | 40.00 | 40.00 | 18.18 | 1.82 | 8 |
| A26 | 30.00 | 30.00 | 39.60 | 0.40 | 4 |
| A27 | 20.00 | 20.00 | 59.41 | 0.59 | 4 |
| A28 | 40.00 | 40.00 | 18.18 | 1.82 | 12 |
| A29 | 10.00 | 10.00 | 79.84 | 0.16 | 8 |
| A30 | 20.00 | 20.00 | 54.55 | 5.45 | 12 |
| A31 | 30.00 | 30.00 | 36.36 | 3.64 | 8 |
| A32 | 10.00 | 10.00 | 79.20 | 0.80 | 4 |

TABLE 2
Formulation details for Top 32 LNPs in
Group B with DDAB as the helper lipid

| | Molar % | | | | |
|---|---|---|---|---|---|
| Code | DDAB | Dlin-MC3 | Chol | DMG-PEG | N/P Ratio |
| B1 | 1.57 | 78.43 | 19.96 | 0.04 | 12 |
| B2 | 7.27 | 72.73 | 19.96 | 0.04 | 8 |
| B3 | 0.59 | 59.41 | 39.92 | 0.08 | 12 |
| B4 | 7.27 | 72.73 | 18.18 | 1.82 | 12 |
| B5 | 7.27 | 72.73 | 19.96 | 0.04 | 12 |
| B6 | 30.00 | 30.00 | 39.60 | 0.40 | 8 |
| B7 | 30.00 | 30.00 | 39.92 | 0.08 | 4 |
| B8 | 5.45 | 54.55 | 39.92 | 0.80 | 12 |
| B9 | 30.00 | 30.00 | 39.60 | 0.40 | 12 |
| B10 | 1.18 | 58.82 | 39.92 | 0.08 | 12 |
| B11 | 20.00 | 20.00 | 59.41 | 0.59 | 12 |
| B12 | 10.00 | 10.00 | 79.21 | 0.79 | 8 |
| B13 | 20.00 | 20.00 | 59.41 | 0.59 | 8 |
| B14 | 0.40 | 79.60 | 19.96 | 0.04 | 12 |
| B15 | 5.45 | 54.55 | 39.60 | 0.40 | 4 |
| B16 | 40.00 | 40.00 | 19.96 | 0.04 | 4 |
| B17 | 0.40 | 79.60 | 19.80 | 0.20 | 8 |
| B18 | 20.00 | 20.00 | 59.41 | 0.59 | 4 |
| B19 | 20.00 | 20.00 | 59.88 | 0.12 | 4 |
| B20 | 40.00 | 40.00 | 19.96 | 0.04 | 12 |
| B21 | 0.40 | 79.60 | 19.80 | 0.20 | 4 |
| B22 | 0.78 | 39.22 | 59.41 | 0.59 | 4 |
| B23 | 10.00 | 10.00 | 79.21 | 0.79 | 4 |
| B24 | 30.00 | 30.00 | 39.60 | 0.40 | 4 |
| B25 | 40.00 | 40.00 | 19.80 | 0.20 | 4 |
| B26 | 40.00 | 40.00 | 19.80 | 0.20 | 8 |
| B27 | 0.40 | 79.60 | 18.18 | 1.82 | 12 |
| B28 | 1.18 | 58.82 | 39.60 | 0.40 | 4 |
| B29 | 40.00 | 40.00 | 19.96 | 0.04 | 8 |
| B30 | 40.00 | 40.00 | 19.80 | 0.20 | 12 |
| B31 | 10.00 | 10.00 | 79.84 | 0.16 | 4 |
| B32 | 7.27 | 72.73 | 19.80 | 0.20 | 4 |

TABLE 3
Formulation details for Top 32 LNPs in
Group C with DOPE as the helper lipid

| | Molar % | | | | |
|---|---|---|---|---|---|
| Code | DOPE | Dlin-MC3 | Chol | DMG-PEG | N/P Ratio |
| C1 | 30.00 | 30.00 | 39.92 | 0.08 | 12 |
| C2 | 30.00 | 30.00 | 39.92 | 0.08 | 8 |
| C3 | 5.45 | 54.55 | 39.60 | 0.40 | 12 |
| C4 | 40.00 | 40.00 | 19.96 | 0.04 | 8 |
| C5 | 7.27 | 72.73 | 19.96 | 0.04 | 12 |
| C6 | 7.27 | 72.73 | 19.80 | 0.20 | 12 |
| C7 | 40.00 | 40.00 | 19.96 | 0.04 | 12 |
| C8 | 30.00 | 30.00 | 39.60 | 0.40 | 12 |
| C9 | 30.00 | 30.00 | 39.60 | 0.40 | 8 |
| C10 | 30.00 | 30.00 | 39.60 | 0.40 | 4 |
| C11 | 1.57 | 78.43 | 19.80 | 0.20 | 12 |
| C12 | 1.57 | 78.43 | 19.96 | 0.04 | 12 |
| C13 | 0.79 | 79.21 | 19.80 | 0.20 | 12 |
| C14 | 40.00 | 40.00 | 19.80 | 0.20 | 8 |
| C15 | 20.00 | 20.00 | 59.41 | 0.60 | 4 |
| C16 | 7.27 | 72.73 | 18.18 | 1.82 | 12 |
| C17 | 40.00 | 40.00 | 18.18 | 1.82 | 4 |
| C18 | 40.00 | 40.00 | 19.80 | 0.20 | 8 |
| C19 | 0.78 | 39.22 | 59.41 | 0.59 | 12 |
| C20 | 40.00 | 40.00 | 19.80 | 0.20 | 4 |
| C21 | 10.00 | 10.00 | 79.21 | 0.79 | 12 |
| C22 | 30.00 | 30.00 | 39.92 | 0.08 | 8 |
| C23 | 0.40 | 79.60 | 19.80 | 0.20 | 12 |
| C24 | 20.00 | 20.00 | 59.41 | 0.59 | 4 |
| C25 | 20.00 | 20.00 | 59.41 | 0.59 | 12 |
| C26 | 40.00 | 40.00 | 19.96 | 0.04 | 4 |
| C27 | 10.00 | 10.00 | 79.21 | 0.79 | 4 |
| C28 | 40.00 | 40.00 | 18.18 | 1.82 | 8 |
| C29 | 30.00 | 30.00 | 36.36 | 3.64 | 12 |
| C30 | 40.00 | 40.00 | 18.18 | 1.82 | 12 |
| C31 | 30.00 | 30.00 | 36.36 | 3.64 | 8 |
| C32 | 20.00 | 20.00 | 54.55 | 5.45 | 12 |

TABLE 4
Formulation details for Top 32 LNPs in
Group D with DSPC as the helper lipid

| | Molar % | | | | |
|---|---|---|---|---|---|
| Code | DSPC | Dlin-MC3 | Chol | DMG-PEG | N/P Ratio |
| D1 | 7.27 | 72.73 | 19.96 | 0.04 | 8 |
| D2 | 7.27 | 72.73 | 19.80 | 0.20 | 8 |
| D3 | 5.45 | 54.55 | 39.92 | 0.08 | 4 |
| D4 | 7.27 | 72.73 | 19.96 | 0.04 | 12 |
| D5 | 3.64 | 36.36 | 59.88 | 0.12 | 4 |
| D6 | 7.27 | 72.73 | 19.96 | 0.04 | 4 |
| D7 | 7.27 | 72.73 | 19.80 | 0.20 | 4 |
| D8 | 3.64 | 36.36 | 59.88 | 0.12 | 8 |
| D9 | 5.45 | 54.55 | 39.60 | 0.40 | 8 |
| D10 | 5.45 | 54.55 | 39.60 | 0.40 | 12 |
| D11 | 3.64 | 36.36 | 59.41 | 0.59 | 4 |
| D12 | 3.64 | 36.36 | 59.41 | 0.59 | 12 |
| D13 | 3.64 | 36.36 | 59.41 | 0.59 | 8 |
| D14 | 5.45 | 54.55 | 36.36 | 3.64 | 8 |
| D15 | 5.45 | 54.55 | 39.60 | 0.40 | 4 |
| D16 | 0.78 | 39.22 | 59.41 | 0.59 | 4 |
| D17 | 20.00 | 20.00 | 59.88 | 0.12 | 8 |
| D18 | 0.40 | 39.60 | 59.41 | 0.59 | 12 |
| D19 | 1.18 | 58.82 | 39.60 | 0.40 | 8 |
| D20 | 7.27 | 72.73 | 18.18 | 1.82 | 4 |
| D21 | 0.40 | 39.60 | 59.41 | 0.59 | 4 |
| D22 | 0.10 | 19.90 | 79.21 | 0.79 | 4 |
| D23 | 1.82 | 18.18 | 79.21 | 0.79 | 12 |
| D24 | 1.82 | 18.18 | 79.21 | 0.79 | 4 |
| D25 | 20.00 | 20.00 | 59.41 | 0.59 | 8 |
| D26 | 20.00 | 20.00 | 59.88 | 0.12 | 12 |
| D27 | 1.82 | 18.18 | 79.84 | 0.16 | 4 |
| D28 | 1.82 | 18.18 | 79.21 | 0.79 | 8 |
| D29 | 1.18 | 58.82 | 36.36 | 3.64 | 8 |
| D30 | 0.20 | 19.80 | 79.21 | 0.79 | 4 |
| D31 | 20.00 | 20.00 | 59.41 | 0.59 | 4 |
| D32 | 20.00 | 20.00 | 59.41 | 0.59 | 12 |

TABLE 5
Formulation details for Top 32 LNPs in
Group E with 14PA as the helper lipid

| | Molar % | | | | |
|---|---|---|---|---|---|
| Code | 14PA | Dlin-MC3 | Chol | DMG-PEG | N/P Ratio |
| E1 | 0.20 | 39.80 | 59.88 | 0.12 | 12 |
| E2 | 5.45 | 54.55 | 39.60 | 0.40 | 8 |
| E3 | 0.40 | 79.60 | 19.80 | 0.20 | 12 |
| E4 | 7.27 | 72.73 | 18.18 | 1.82 | 12 |
| E5 | 7.27 | 72.73 | 19.96 | 0.04 | 12 |
| E6 | 20.00 | 20.00 | 59.41 | 0.59 | 4 |
| E7 | 30.00 | 30.00 | 39.92 | 0.08 | 8 |
| E8 | 1.18 | 58.82 | 39.60 | 0.40 | 12 |
| E9 | 7.27 | 72.73 | 19.80 | 0.20 | 4 |
| E10 | 30.00 | 30.00 | 39.92 | 0.08 | 4 |
| E11 | 7.27 | 72.73 | 19.96 | 0.04 | 8 |
| E12 | 40.00 | 40.00 | 19.96 | 0.04 | 8 |
| E13 | 1.81 | 18.18 | 79.21 | 0.79 | 8 |
| E14 | 5.45 | 54.55 | 39.60 | 0.40 | 4 |
| E15 | 40.00 | 40.00 | 19.80 | 0.20 | 4 |
| E16 | 1.57 | 78.43 | 19.80 | 0.20 | 8 |
| E17 | 0.10 | 19.90 | 79.84 | 0.16 | 4 |
| E18 | 20.00 | 20.00 | 59.88 | 0.12 | 4 |
| E19 | 3.64 | 36.36 | 59.88 | 0.12 | 8 |
| E20 | 1.57 | 78.43 | 19.96 | 0.04 | 8 |
| E21 | 40.00 | 40.00 | 19.96 | 0.04 | 12 |
| E22 | 3.64 | 36.36 | 59.41 | 0.59 | 12 |
| E23 | 30.00 | 30.00 | 39.60 | 0.40 | 4 |
| E24 | 0.30 | 59.70 | 39.92 | 0.08 | 12 |
| E25 | 1.82 | 18.18 | 79.21 | 0.79 | 8 |
| E26 | 30.00 | 30.00 | 39.60 | 0.40 | 8 |
| E27 | 0.30 | 59.70 | 39.92 | 0.08 | 8 |
| E28 | 40.00 | 40.00 | 19.80 | 0.20 | 12 |
| E29 | 1.82 | 18.18 | 79.21 | 0.79 | 4 |
| E30 | 7.27 | 72.73 | 19.80 | 0.20 | 8 |
| E31 | 1.57 | 78.43 | 19.80 | 0.20 | 12 |
| E32 | 7.27 | 72.73 | 19.96 | 0.04 | 4 |

TABLE 6
Formulation details for Top 32 LNPs in
Group F with 18PG as the helper lipid

| | Molar % | | | | |
|---|---|---|---|---|---|
| Code | 18PG | Dlin-MC3 | Chol | DMG-PEG | N/P Ratio |
| F1 | 0.20 | 39.80 | 59.41 | 0.59 | 4 |
| F2 | 0.40 | 39.60 | 59.41 | 0.59 | 4 |
| F3 | 0.20 | 39.80 | 59.41 | 0.59 | 8 |
| F4 | 0.59 | 59.41 | 39.60 | 0.40 | 4 |
| F5 | 5.45 | 54.55 | 39.60 | 0.40 | 12 |
| F6 | 1.18 | 58.82 | 39.92 | 0.08 | 12 |
| F7 | 0.20 | 39.80 | 54.55 | 5.45 | 8 |
| F8 | 0.78 | 39.22 | 59.88 | 0.12 | 4 |
| F9 | 3.64 | 36.36 | 59.88 | 0.12 | 4 |
| F10 | 0.20 | 39.80 | 59.88 | 0.12 | 8 |
| F11 | 0.30 | 59.70 | 39.92 | 0.08 | 8 |
| F12 | 7.27 | 72.73 | 19.96 | 0.04 | 8 |
| F13 | 0.40 | 39.60 | 59.41 | 0.59 | 12 |
| F14 | 3.64 | 36.36 | 59.41 | 0.59 | 12 |
| F15 | 0.40 | 39.60 | 59.41 | 0.59 | 8 |
| F16 | 0.20 | 39.80 | 59.41 | 0.59 | 12 |
| F17 | 0.30 | 59.70 | 39.60 | 0.40 | 4 |
| F18 | 0.30 | 59.70 | 39.60 | 0.40 | 8 |
| F19 | 0.30 | 59.70 | 39.60 | 0.40 | 12 |
| F20 | 1.18 | 58.82 | 39.60 | 0.40 | 4 |
| F21 | 30.00 | 30.00 | 39.60 | 0.40 | 4 |
| F22 | 20.00 | 20.00 | 59.88 | 0.12 | 4 |
| F23 | 5.45 | 54.55 | 39.92 | 0.08 | 8 |
| F24 | 5.45 | 54.55 | 39.92 | 0.08 | 12 |
| F25 | 1.82 | 18.18 | 79.84 | 0.16 | 8 |
| F26 | 1.57 | 78.43 | 19.96 | 0.04 | 8 |
| F27 | 0.59 | 59.41 | 39.60 | 0.40 | 12 |
| F28 | 1.57 | 78.43 | 19.80 | 0.20 | 4 |
| F29 | 7.27 | 72.73 | 19.80 | 0.20 | 8 |
| F30 | 0.79 | 79.21 | 19.80 | 0.20 | 12 |
| F31 | 1.57 | 78.43 | 19.96 | 0.04 | 4 |
| F32 | 1.82 | 18.18 | 79.84 | 0.16 | 12 |

### Example 5

Compositional Optimization of mRNA Lipid Nanoparticles to Modulate Th1/Th2 Immune Activation Profile and Potentiate Anticancer Immunity

### 5.1 Overview

Lipid nanoparticles (LNPs) have been successfully designed as immunostimulatory delivery platforms for antigen-encoding mRNA for cancer immunotherapy. Primary efforts have been focusing on engineering LNPs to promote transfection and maturation of antigen-presenting cells, and modulate the TLR-mediated adjuvant activity to potentiate CD8+ T cell response and antitumor efficacy. Here, we employed a multi-step screening method to optimize the type of helper lipid and component ratios in an mRNA LNP library to efficiently deliver antigen-encoding mRNA and modulate the immune activation profile, i.e., the balance between Th1 and Th2 responses. By first screening for dendritic cell maturation and antigen presentation in vitro followed by assessing immune activation and suppression of tumor growth in vivo, we identified LNP formulations with potent antitumor efficacy, especially in combination with immune checkpoint blockade therapy. Our results further revealed that LNPs showing strong dual immunostimulation activity in both Type-1 T helper (Th1) and Th2 responses generated the most potent antitumor efficacy by eliciting a coordinated attack by dual T cells and NK cells. These findings highlight the critical role of LNP composition in addition to payload in tailoring immune responses and afford new opportunities to optimize RNA-based cancer and infectious disease treatments.

### 5.2 Background

Successful deployment of two mRNA vaccines, Spikevax® (Moderna) and Comirnaty® (BioNTech/Pfizer), against SARS-COV-2 in response to the outbreak of the coronavirus pandemic has clearly validated the safety and efficacy of lipid nanoparticle (LNP) delivery platform for mRNA vaccine modality. Huang et al., 2021; Huang et al., 2022; Wouters et al., 2021. As a potent vehicle mediating expression of mRNA encoding antigen of interest, LNPs have been shown to elicit strong antibody and memory B-cell responses. Sahin et al., 2021. In this case, the antigen is primarily expressed by cells at the injection site and then internalized and processed by antigen presenting cells (APCs), such as dendritic cells (DCs) to generate strong antibody and T helper cell responses. Sahin et al., 2021; Wang et al., 2021; Lederer et al., 2020; Alameh et al., 2021; Turner et al., 2021; Hou et al., 2021; Sahin et al., 2014; Pardi et al., 2018; Kowalski et al., 2019.

The strong potency has been attributed to the adjuvant activity of LNPs, particularly their ability to induce germinal center formation and T follicular helper (Tfh) cell response. Wang et al., 2021; Lederer et al., 2020; Alameh et al., 2021. In addition, evidence of generated IFNγ⁺or IL2⁺CD8⁺ T cells, as well as CD4⁺ Th1 cells, also were reported from the mRNA LNP vaccine. Sahin et al., 2021. These reports support the notion that LNPs optimized for COVID-19 mRNA vaccines may also generate CD4⁺ Th1 and CD8⁺ cell-mediated cellular immunity, Laczkó et al., 2020, which may contribute to the Th2-response required for a strong humoral response to generate a high level of neutralizing antibodies. Painter et al., 2021; Lozano-Rodríguez et al., 2022; Reichmuth et al., 2016; Hassett et al., 2021; Pilkington et al., 2021; Miao et al., 2021.

Although Th2 and Th17 responses are essential to generate a potent humoral response and eradicate the extracellular pathogens, Sankaradoss et al., 2022; Bretscher, 2014; Bretscher, 2019; Del Prete, 1998, an increasing number of reports have demonstrated that supplementing a strong Th2 response with a Th1-mediated cellular immunity not only can help to clear SARS-COV-2-infected cells, but also potentiated the humoral response. Duarte et al., 2021; Kyriakidis et al., 2021; Zhang et al., 2021; Mulligan et al., 2020; Polack et al., 2020.

LNPs have been tested previously for the delivery of mRNA vaccines to treat cancer and prevent other infections, Alameh et al., 2020, including Zika virus, Richner et al., 2017, influenza, Lindgren et al., 2017, flavivirus, VanBlargan et al., 2018, HIV, Pardi et al, 2019, and the like.

It has become evident that the induction of a potent antigen-specific immune response requires a specifically tailored immune activation profile. When delivered antigen is expressed in DCs or other APCs, it generates peptide epitopes loaded and displayed in the context of MHC class I molecules, leading to activation of CD8⁺ T cells. Hou et al., 2021; Sahin et al., 2014; Pardi et al, 2018; Kowalski et al, 2019; Sahin et al., 2020; Miao et al., 2019; Oberhardt et al., 2021; Oberli et al., 2017; Karmacharya et al., 2022; Guevara et al., 2020.

A strong cytotoxic CD8⁺ T cell response and a Type-1 T helper cell (Th1) immune response are critical to the design of an effective tumor vaccine, leading to the clearance of intracellular pathogens and cancer cells. Miao et al., 2019. Studies have shown that LNP formulations can be identified to promote transfection and maturation of DCs, macrophages and neutrophils and modulate the TLR-mediated adjuvant activity, for the purpose of potentiating CD8+ T cell response and antitumor efficacy. The correlation among the composition, therapeutic efficacy, and the immune activation profile of mRNA-LNPs, particularly the balance between Th1 and Th2 responses, and coordination with NK cell-mediated cell killing, however, remains elusive.

LNP systems offer distinct advantages in terms of the structural versatility offered by diverse lipid compositions and broad transfection capability across a wide range of cell populations. Cullis and Hope, 2017; Zhang et al., 2021; Lokugamage et al., 2021; Wang et al., 2015; Cheng et al., 2020; Dilliard et al. 2021; Patel et al., 2019; Patel et al., 2022; Swingle et al., 2022; Zhu et al., 2022. Most LNP-based nucleic acid delivery platforms that are commercially available or investigated in clinical studies consist of four or five lipid components: a helper phospholipid (e.g., 1,2-distearoyl-sn-glycero-3-phosphocholine (DSPC)), an ionizable lipid, cholesterol, a PEGylated lipid, and a selective organ targeting lipid. Cheng et al., 2020; Li et al., 2015; Cheng and Lee, 2016; Kulkarni et al., 2017.

Recent studies have reported that the choice of lipid components and relative proportions of the lipid ingredients in the formulation, greatly influence in vivo transfection efficiency and tissue-specific delivery. Cheng et al., 2020; Dilliard et al., 2021; Zhu et al., 2022; Dobrowolski et al., 2022; Xue et al., 2022. Despite the recent advance of mRNA LNP-based vaccines, there is a lack of in-depth analysis on the effect of helper lipid charge and the relative ratios of the LNP components on the transfection of different cell populations at the site of administration, which may play an important role in determining antigen expression levels in APCs, immune activation profile, and therapeutic effects. Without wishing to be bound to any one particular theory, it was thought that a distinct immune activation profile may be generated by tailoring LNP composition to modulate transfected cell populations.

Herein, we screened 1,080 LNP formulations for transfection efficiency APCs with a goal of inducing a robust cellular immunity and identified a cohort of formulations with the highest transfection efficiency in bone-marrow-derived dendritic cells (BMDCs) and antigen presentation ability. The selected formulations were further examined for the transgene expression levels and immune response induction following subcutaneous (s.c.) or intramuscular (i.m.) injection. We showed the ability to tune the balance of immune stimulation between Th1- and Th2-immune responses by altering the LNP compositions. We also explore the feasibility of using one LNP formulation to elicit both strong Th1- and Th2-immune responses, and the effect of such a dual attack in improving the antitumor efficacy compared to formulations with Th1-biased immune responses alone. Furthermore, we confirmed the synergistic effect of combining the optimized mRNA LNP vaccine and systemic immune checkpoint blockade therapy in melanoma therapeutic models; and investigated the mechanisms underlying the enhanced immune response and correlations with cellular transfection activity and local immune activation profiles.

### 5.3 Results

5.3.1 LNP Screening for mRNA Delivery to Dendritic Cells, Antigen Presentation and Maturation

To generate a diverse 1,080 member LNP library, we used DLin-MC3-DMA as the ionizable lipid, DMG-PEG2000 as the PEGylated lipid, and six helper phospholipids that were previously used in FDA-approved or experimental LNP formulations. Zhu et al., 2022. These were chosen to represent a range of different charges: cationic lipids: 1,2-dioleoyl-3-trimethylammonium-propane (DOTAP) and dimethyldioctadecyl ammonium (DDAB); zwitterionic lipids: 1,2-dioleoyl-sn-glycero-3-phosphoethanolamine (DOPE) and DSPC; and anionic lipids: 1,2-dimyristoyl-sn-glycero-3-phosphate (14PA) and 1-stearoyl-2-oleoyl-sn-glycero-3-phospho-(l′-rac-glycerol) (18PG). Cheng et al., 2020; Dilliard et al., 2021; Patel et al., 2022; Cheng and Lee, 2016; Dobrowolski et al., 2022; Xue et al., 2022; Zhang et al., 2022.

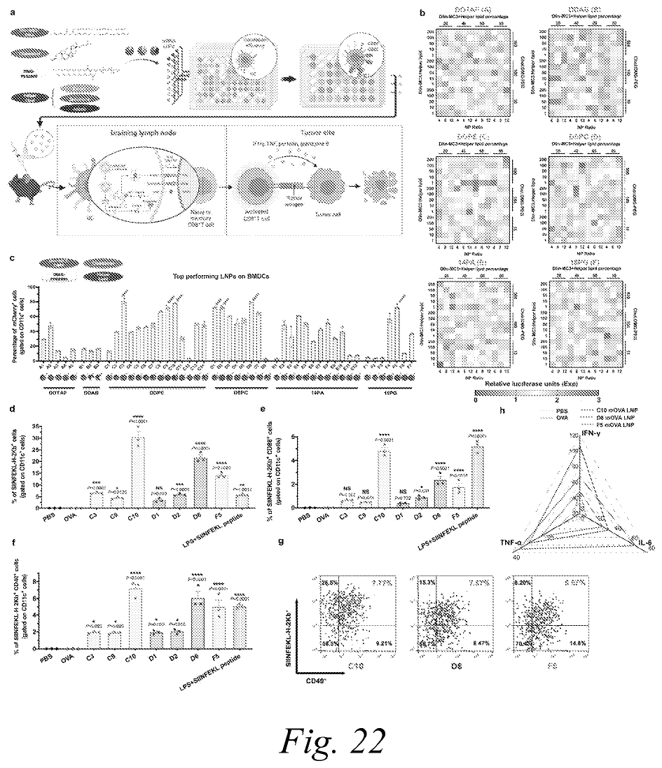

Using DLin-MC3-DMA, cholesterol, DMG-PEG2000, and one of the six helper lipids, the final 1,080 LNP formulations were generated by varying the following parameters: (1) combined molar percentage of DLin-MC3-DMA and helper lipid ranging from 20% to 80%; (2) weight ratio of cholesterol to DMG-PEG2000 ranging from 10 to 500; (3) weight ratio of DLin-MC3-DMA to helper lipid ranging from 1 to 200; and (4) the molar ratio of chargeable groups in ionizable lipid to phosphate groups in pDNA (N/P ratio) ranging from 4 to 12. These parameter choices provided us with a sufficiently diverse library of LNP formulations from which we assessed for mRNA delivery (FIG. 22a).

To select LNP formulations with strong APC-specific transgene expression, we first evaluated the mRNA delivery efficiency of the LNP library in DC2.4 cells (an immortalized murine dendritic cell line) using firefly luciferase (fLuc) mRNA and measured luciferase protein expression (FIG. 22b). With the helper lipid fixed, adjusting the above-mentioned four parameters in the LNP formulations significantly varied the gene expression levels. Next, we validated the transfection efficiency of the top 49 LNP formulations containing mCherry mRNA in BMDCs using flow cytometry analysis (Table 7). Results shown in FIG. 22c confirmed the high in vitro transfection efficiency of these LNPs. Seven LNP formulations, including C3, C9, C10, D1, D2, D6, F5 (indicated by red arrows) achieved potent transfection of BMDCs with more than 70% of cells transfected (Table 8).

For effective mRNA vaccines, both efficient antigen expression and potent immune cell activation are needed to generate a robust immune response. The immunostimulatory effect of the seven selected formulations were therefore tested on BMDCs using mRNA encoding ovalbumin (mOVA), a model antigen protein widely used in vaccine studies. After treated with mOVA LNP for 72 h, compared with PBS, OVA protein, and other formulations, C10, D6 and F5 LNPs resulted in a markedly elevated expression of the OVA-derived SIINFEKL peptide on MHC-I, indicating the successful antigen presentation (approximately 30% SIINFEKL-H-2Kb⁺for C10, approximately 21.5% for D6 and approximately 14.0% for F5) (FIG. 22d). In addition, a significantly higher level of CD86⁺SIINFEKL-H-2Kb⁺ and CD40⁺SIINFEKL-H-2Kb⁺was observed after treatment with these three LNPs (FIG. 22e-FIG. 22g). Compared with the positive control group treated with lipopolysaccharides (LPS) and SIINFEKL peptide, F5 successfully induced a comparable level of CD40⁺SIINFEKL-H-2Kb⁺ (approximately 5.0%), and C10 and D6 generated 1.43-fold and 1.22-fold higher levels, respectively. Along with an improved maturation level of BMDCs, significantly higher secretion levels of inflammatory cytokines IFN-γ, TNF-α and IL-6 also were observed within the supernatants of BMDCs after treatment with the three OVA mRNA-loaded LNPs (C10, D6 and F5 LNPs) (FIG. 22h).

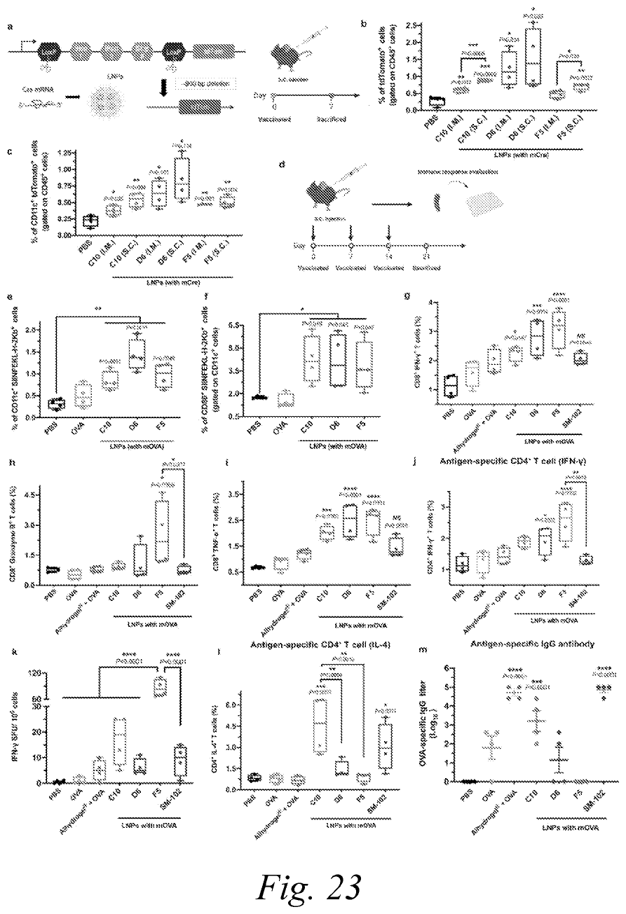

We next evaluated the in vivo delivery efficacy of these candidates in mice following s.c. injection or i.m. injection. To determine whether transfected APCs were present within draining lymph nodes, we delivered Cre-recombinase mRNA (mCre) LNPs to genetically engineered tdTomato (tdTom) reporter mice (Ai9 mice) containing a LoxP-flanked stop cassette that prevents expression of the tdTom protein. This mouse model allows detection of the transfected cells as a result of Cre recombinase expression (FIG. 23a), as the expressed recombinase edits out the stop cassette enabling the expression of fluorescent tdTom. Our results indicated that all three LNP candidates (C10, D6 and F5) yielded significant levels of tdTom+ lymphocytes and DCs in the draining lymph nodes (FIG. 23b, FIG. 23c). Consistent with many reports showing that nanovaccines can efficiently traffic to the draining lymph node after s.c. administration, our results indicated that compared with i.m. injection, a higher percentage of transfected cells was detected in the draining lymph nodes following s.c. injection (FIG. 23b, c). Based on this finding, the s.c. administration route was selected for further testing.

Next, the antigen presentation and maturation levels of APCs in draining lymph nodes were examined after s.c. injection of LNPs (FIG. 23d). After three dosages of mRNA LNPs, all three formulations significantly improved the SIINFEKL-H-2K^{b+} DC level in the draining lymph nodes; 2.9-, 2.0- and 1.7-fold higher levels of SIINFEKL-H-2K^{b+} DCs were observed for D6, F5, and C10, respectively, compared to free OVA protein treated group (FIG. 23e). The abundance of CD86⁺SIINFEKL-H-2K^{b+} DCs also was increased by 2.7-, 2.5-, and 2.8-fold after treatment with D6, F5 and C10, respectively, compared to the free OVA protein treated group (FIG. 23f).

Taken together, the above results demonstrated the successful identification of the top 49 formulations from an LNP library consisting of 1,080 formulations based on the transfection efficiency in DC2.4 cells. Further evaluations on BMDCs led to the selection of seven leading LNPs, and three of them (C10, D6, and F5 LNPs) showed potent antigen presentation and immunostimulatory effects both in vivo and in vitro. These three leading formulations were thus selected for further in vivo tests.

### 5.3.2 Distinct Immunological Profiles of Top-Performing LNPs

The vaccination potential of the three lead LNPs was further tested in mice following s.c. injections (three dosages at days 0, 7 and 14) (FIG. 23d). For comparison, we also included SM-102 LNP formulation used in Moderna COVID-19 vaccine (Spikevax®) and classic adjuvant aluminum hydroxide gel (Alhydrogel®) (mixed with OVA protein at 1:1 ratio) in further experiments. The antigen specific CD8⁺ T cell response induced by LNPs was first assessed. Spleens of vaccinated mice were harvested on day 21 and homogenized into a cell suspension for ex vivo antigen restimulation. Increased frequencies of CD8 IFN-γ⁺, CD8⁺Granzyme B⁺, and CD8⁺ TNF-α⁺cell populations were observed for all three LNP-treated groups (FIG. 23g-FIG. 23i). Compared to the free OVA protein treated group, there were approximately 2.3-, 2.9- and 2.9-fold increases after treated with C10, D6, and F5 LNPs, respectively, in terms of CD8 TNF-α⁺ cell frequencies. The limited increase of antigen-specific cytotoxic T cells was observed for both Alhydrogel® and SM-102 LNP treated groups. Compared to SM-102 LNP, there were approximately 1.4-, 1.8-, and 1.8-fold increases after treated with C10, D6 and F5 LNPs, respectively, in terms of CD8⁺ TNF-α⁺ cell frequencies. Along with the potent CD8⁺ T cell response, a significantly higher numbers of Th1 cells (CD4⁺IFN-γ⁺) also were observed for all three formulations (FIG. 23j). Especially for F5 LNPs, there was an approximately 2.0-fold higher level of antigen-specific Th1 cells after vaccination, and 1.5-fold increase for both C10 and D6 formulations. In contrast, there was no significant increase observed for alhydrogel and SM-102 LNP treated groups. Compared to SM-102 LNP, there were approximately 1.3-, 1.4-, and 1.8-fold increases in terms of level of antigen-specific Th1 cells after treated with C10, D6, and F5 LNPs, respectively. The elevated production of proinflammatory cytokines after restimulation with antigen, including IFN-γ, TNF-α, and IL-6, also indicates a potent antigen-specific Th1-type response induced by the three leading LNP formulations (FIG. 2k).

The frequency of antigen-specific CD4⁺IL-4⁺ Th2 cells and the magnitude of antibody responses generated were further examined for these groups. Compared to the free OVA protein treated group, significantly more Th2 cells (5.8-fold higher, P=0.0003) were observed in the C10-treated group (FIG. 23l), in contrast to D6 and F5 groups (1.8- and 1.1-fold higher, respectively; P>0.5). In addition, as shown in FIG. 2m, C10 induced a significantly higher OVA-specific IgG titer including both IgG1 and IgG2a subclass titers, indicating a potent humoral response. However, the antibody response generated by D6 was limited, and it was undetectable for F5. For both Alhydrogel® and SM-102 LNP groups, potent OVA-specific IgG titers were observed at 3 weeks after vaccination.

The above results indicate that after vaccination all three selected LNPs successfully induced a more potent antigen-specific Th1-type immune response than Alhydrogel® and SM-102 LNPs. For the Th2-type response, however, the three leading LNP formulations exhibited distinct profiles. C10 LNPs elicited both potent Th1 and Th2 responses. While D6 and F5 generated a potent Th1 response, they generated moderate or undetectable levels of Th2 responses.

### 5.3.3 Differential Anti-Tumor Effects Among the Top Performing LNPs

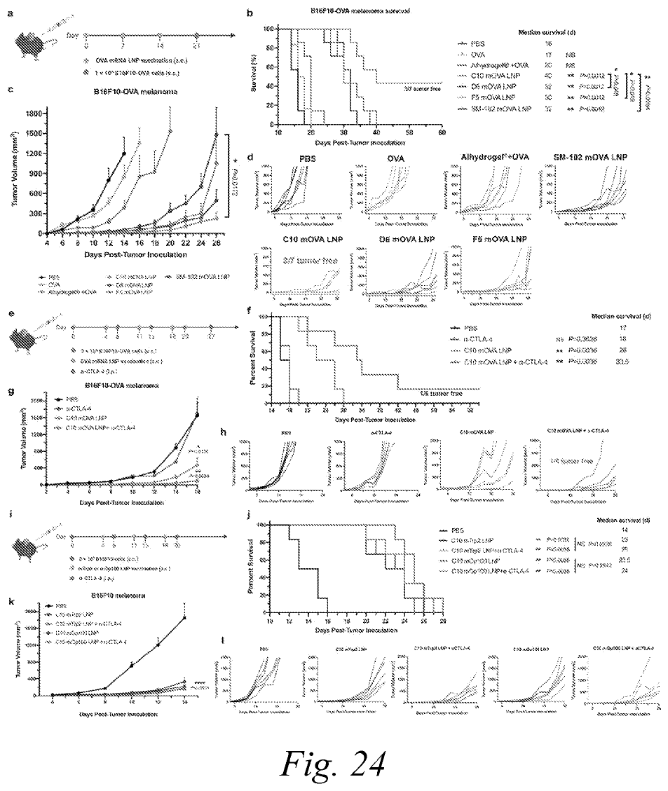

Given the potent antigen-specific immune responses induced by the top mRNA LNPs, we examined the efficacy of the three LNP formulations as cancer vaccines in multiple prophylactic and therapeutic tumor models. C57BL/6 mice were immunized on days 0, 7, and 14 with 10 μg of free OVA protein, OVA protein mixed with Alhydrogel® or LNPs containing 10 μg of mOVA. On day 21, animals were inoculated s.c. in the right posterior side with 1×10⁶ B16F10-OVA cells (FIG. 24a). All three LNP formulations along with SM-102 LNPs showed significantly stronger tumor growth inhibition rates with prolonged overall survival times than free OVA protein and Alhydrogel® group. The median survival time was 40, 32, 30, and 32 days for C10, D6, F5 and SM-102 LNPs, respectively, compared to 15 days for the free OVA protein group and 20 days for Alhydrogel® group (FIG. 24b-FIG. 24d). Furthermore, compared to D6 and F5, which generated strong Th1 response only, or SM-102 LNP, which generated strong Th2 response only, C10 LNPs triggering both Th1 and Th2 responses yielded a markedly improved protection effect with around 40% of the mice remaining tumor free beyond 60 days.

We next assessed the therapeutic efficacy of C10 LNPs as a therapeutic vaccine in the B16F10 tumor model using the model OVA antigen, as well as two other clinically relevant antigens tyrosinase-related protein 2 (Trp2) and glycoprotein 100 (Gp100). First, C57BL/6 mice were inoculated s.c. in the right posterior side with 3×10⁵ B16F10-OVA cells on day 0. On days 4, 11, and 18, the mice were vaccinated with C10 LNPs containing 10 μg mOVA (FIG. 24e). As demonstrated in FIG. 24f-FIG. 24h shows, the C10 LNPs showed a significant tumor suppression effect in this treatment model with a median survival time of 26 days compared to 16 days for the negative control group. When C10 was given in combination with an immune checkpoint inhibitor (100 μg anti-CTLA-4 monoclonal antibody, given i.p. on days 6, 13, 20 and 27), a synergistic effect was observed with a prolonged median survival time of 33.5 days. In contrast, no significant tumor suppression effect was observed for the group treated with only α-CTLA-4 antibody in comparison with the PBS control.

The C10 LNPs were next tested in the same mouse model using clinically relevant tumor antigens Trp2 and Gp100 and not the model antigen OVA (FIG. 24i). C57BL/6 mice were inoculated subcutaneously in the right posterior side with 3×10⁵ B16F10 cells on day 0. On days 4, 11, and 18, the mice were vaccinated with C10 LNPs containing 10 μg of mRNA encoding either Trp2 or Gp100. The potent anti-tumor effect also was observed by using these two antigens, showing substantially prolonged median survival times of 23 and 23.5 days for C10-mTrp2 LNPs and C10-mGp100 LNPs, respectively (FIG. 24j-FIG. 24l). However, no significant improvement was observed when combining these C10 formulations with α-CTLA-4 antibody treatment (FIG. 24j-FIG. 24l).

5.3.4 Long-Term Protection Correlated with Dual Attack by T Cells and NK Cells

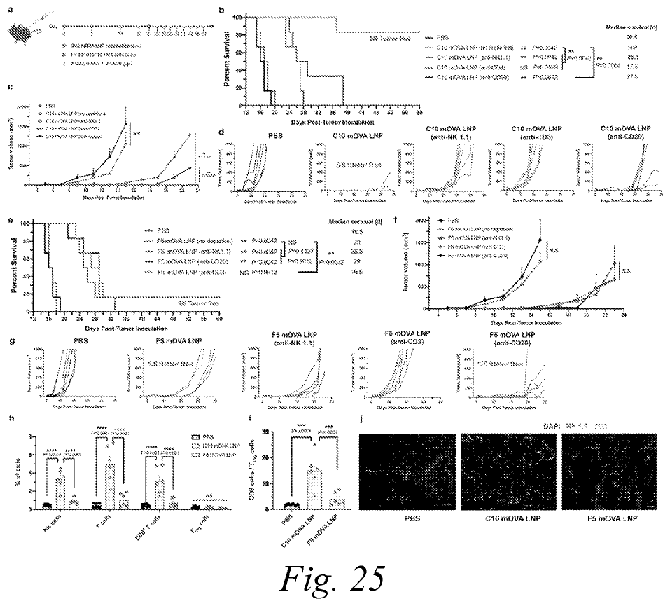

To further understand the mechanisms of mRNA LNP vaccine efficacy, cell depletion experiments were conducted for C10 and F5 LNPs that induced distinct immunological profiles on the B16F10-OVA melanoma model (FIG. 25a). As shown in FIG. 25b-FIG. 25g, depletion of CD3⁺ T cells, NK1.1⁺NK cells, or CD20⁺B cells markedly reduced the survival advantage conferred by C10-mOVA LNPs. In contrast, for F5-mOVA LNPs, the antitumor effect was abolished only when T cells were depleted. Removal of NK cells or CD20⁺B cells did not significantly alter tumor suppression effect induced by F5-mOVA LNPs.

Multiple effector lymphocyte populations were statistically enriched in tumors from mice immunized with C10 LNPs at 22 days post tumor inoculation, with a 6.9-fold enrichment in NK cells, a 11.2-fold enrichment in T cells and a 7.4-fold enrichment in CD8+ T cells compared with the PBS control group (FIG. 25h). In addition, C10-mOVA LNPs established a markedly higher CD8-to-regulatory T cell (T_{reg}) ratio in the tumor: 7.8- and 3.7-fold higher than the PBS group and F5-mOVA LNPs, respectively (FIG. 25i). Furthermore, as shown in FIG. 25j, higher numbers of NK cells and T cells were observed in tumors from the mice treated with C10-mOVA LNPs, whereas no such enrichment effect was detected in mice treated with F5-mOVA LNPs.

### 5.3.5 Potential Pathways for Inducing Th1-Only or Th1-Plus-Th2 Immune Responses

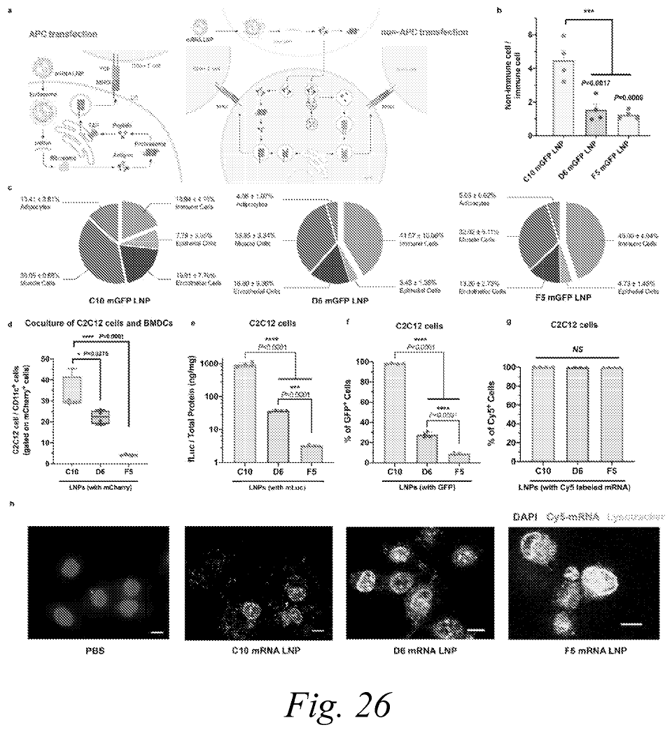

As shown in FIG. 26a, when the antigen is translated in the cytosol of DCs and the processed peptides loaded onto MHC-I molecules, potent cytotoxic T cell response along with Th1 immune response can be initiated. On the other hand, a Th2 response requires the antigen to be expressed and released by non-APCs, and then internalized and processed by DCs, macrophages or B cells to be presented in the context of MHC-II. Although in some circumstances, antigens from the extracellular environment can be presented on MHC class I molecules via cross-presentation pathways, the compositions of mRNA LNPs have limited effect on this process. To explore the mechanism of biased Th1 vs. Th2 responses generated by GFP mRNA containing LNPs with different compositions, we examined the local transfection process following s.c. injection. At 24 h post injection, we quantified GFP expression levels in various cell types at the injection sites using flow cytometry (FIG. 26b-FIG. 26c). After a single injection of D6 or F5 LNPs, around 45% of the transfected cells in the local tissue were immune cells, which was more than two-fold higher than C10 LNPs. The ratio of non-immune cells to immune cells among the GFP-expressing cells was 4.5 in the C10 group, which was 2.9- and 3.6-fold higher than D6 and F5 groups, respectively. These results indicated significant differences among LNPs with different compositions in terms of cell types transfected.

To further investigate the differences between the transfection efficiencies in non-immune cells versus immune cells, we co-cultured the C2C12 cells, a mouse myoblast cell line, and BMDCs (1:1) and quantified in vitro delivery to two cell types using flow cytometry to detect mCherry expression, which revealed that the ratio of C2C12 cell to BMDCs among transfected cells reached as high as 33.41 for C10 LNPs, 22.21 for D6 and only 3.94 for F5 (FIG. 26d). When only C2C12 cells were transfected with LNPs containing fLuc mRNA, the average fLuc expression level in C2C12 cells transfected by C10 was 25- and 289-fold higher than that by D6 and F5 LNPs, respectively (FIG. 26e). When a pure BMDC culture was transfected with LNPs containing mCherry mRNA, however, similar transfection efficiencies were observed for C10, D6, and F5 (FIG. 26f). We further examined the transfection efficiency together with cellular uptake using LNPs containing 50% of mGFP labeled with Cy5 and 50% unlabeled mGFP. While nearly 100% Cy5+ cells (i.e., cellular uptake) were observed for all three groups, only approximately 27.7% and 8.9% of C2C12 cells were transfected by D6 and F5, respectively, in contrast to 98.1% of cells transfected by C10, likely caused by different endosomal escape capabilities among the three LNP formulations in C2C12 myoblasts (FIG. 26g-FIG. 26h). When using Lysotracker to visualize lysosomes of C2C12 cells, the lack of co-localization in fluorescence between LNP (red) and lysosomes (green) were observed for C10 LNP, suggesting successful lysosomal escape and cytosolic delivery (FIG. 26l).

### 5.4 Discussion

The use of LNPs as a non-viral gene carriers has advanced rapidly over the past few years as evidenced by the approval of multiple LNP-based COVID-19 vaccines and one siRNA therapy. Pardi et al., 2018; Akinc et al., 2019. Safety following repeated LNP dosing provides strong momentum for extending the utility of LNPs to therapeutic vaccines and other gene delivery applications. Huang et al., 2021; Sahin et al., 2021; Mulligan et al., 2020. Previous reports on LNP-mediated gene delivery revealed that both the choice of lipids and their molar ratios can drastically influence the encapsulation efficiency of nucleic acid payload, transfection efficiency, and cell/tissue targeting profiles. Lokugamage et al., 2021; Cheng et al., 2020; Zhu et al., 2022.

In this study, we screened LNP compositions to interrogate the role of the carriers themselves in polarizing therapeutic immune responses. We identified three top-performing LNPs from a 1,080-candidate library based on transfection efficiency in BMDCs. These three LNPs induced comparably potent antigen-specific Th1 responses following three doses by s.c. injections but significantly different Th2 responses. All three formulations showed significant levels of efficacy in tumor suppression and markedly prolonged survival in a prophylactic model of OVA-expressing melanoma in C57BL/6 mice. The best candidate, C10 LNPs, however, showed the strongest potency in slowing tumor growth and extending survival when tested in therapeutic melanoma models using mRNA encoding OVA, Trp2 or Gp100 antigens.

Previous reports on cancer vaccine delivery systems demonstrated that a potent Th1 immune response is essential to the anti-tumor efficacy. Badrinath et al., 2022. Using LNP-delivered mRNA vaccine optimized in this study, we revealed that incorporating a strong Th2 response with a potent Th1 response further enhanced the tumor suppression efficiency by comparing the two representative LNP formulations that induce Th1-only or Th1-plus-Th2 immune responses. The results confirmed that coordinated immune responses by various cell populations, including T cells, NK cells, and B cells, generated by C10-mRNA LNPs, provided more effective and comprehensive protection against tumor challenge, and multiple effector cell populations involved in both Th1 and Th2 responses collectively contributed to the long-term protection. T cell-mediated immunity greatly inhibits the growth of the tumors, but many tumors effectively evade the immune system and progress under the immune pressure via multiple mechanisms, including loss of MHC Class I expression and development of an immunosuppressive tumor microenvironment. Our data suggests that with successful induction of a Th2 immune responses, long-term antitumor protection can be achieved by making use of the innate immune cells, such as NK cells, providing antitumor cytotoxicity activated by antibodies linked to target cells. Our data showed that a coordinated action of NK cells and B cells played a critical role in terms of the long-term protection against tumor.

Previous reports on LNP-enabled gene delivery systems argue that LNP composition influences tissue-targeting and transfection. Lokugamage et al., 2021; Cheng et al., 2020; Dilliard et al., 2021; Li et al., 2015. We revealed that, by tuning the composition of LNP formulations, we were able to alter the transgene level delivered by LNP-mediated mRNA vaccine in different cell types in vivo. Further, the biases in cell type specific gene expression were shown to alter immune activation profile of these formulations. LNPs with strong transfection efficiencies in APCs can generate potent cytotoxic T cell and Th1 responses. By varying the composition, LNPs may also show strong transfection efficiency in non-APC cell types, such as myoblasts, thereby aiding Th2 responses. These findings further highlight the need for a rational screening approach when evaluating LNP formulations for antigen-specific therapeutic vaccines and other genetic medicine applications. This study showed that it is feasible to modulate Th1-vs. Th2-biased responses by varying the composition of LNPs to alter their preferential transfection properties in APCs and non-APC cell types. This synthetic tuning approach creates opportunities for devising new mRNA LNP-based immunotherapy strategies that can be intricately tailored to specific disease targets, tissues, and cell types.

The three selected LNP candidates generated different levels of Th2 responses that correlates to different transfection ability in non-APC cells, such as myoblasts. Among them, C10 LNPs with a zwitterionic helper lipid DOPE showed potent Th1-plus-Th2 responses and correlated with their higher transfection activities in both DCs and myoblasts, whereas F5 LNPs with an anionic helper lipid showed strong transfection activity only in DCs with consequently Th1-skewed responses. These results showed that altering the composition of LNP formulations allowed for preferential transfection activity across various cell types, as illustrated here by myoblast cells vs. DCs. Different endosomal escape capabilities of different LNP formulations were likely the determining factor for the difference in transfection efficiency. This cell type-preferential transfection strategy might be a potential tool for modulating the balance of antigen-specific immune stimulation between Th1- and Th2-responses.

Overall, we report a composition screening platform that allowed us to identify the best-performing mRNA LNPs for APC-specific transgene expression that showed a strong Th1-immune response against tumor antigens in a melanoma mouse model. Among the top LNP candidates, C10 showed both potent Th1 and Th2 responses that further enhanced therapeutic efficacy compared with a Th1-skewed response against melanoma antigens. The data indicate that coordinated T cell, NK cell, and B cell responses were responsible for enhanced antitumor efficacy. In addition, tunning the composition of LNP formulations altered the transgene level delivered by LNP-mediated mRNA vaccines in different cell types in vivo. This study thus demonstrated a potential strategy to tailor antigen-specific immune activation profiles generated by tuning LNP composition, providing a versatile vaccine development platform that can be applied to a variety of diseases and leveraged to expand the utility of mRNA LNP-based immunotherapies.

### 5.5 Materials and Methods

### 5.5.1 Materials

DLin-MC3-DMA was purchased from MedKoo Biosciences. DSPC, DOPE, DOTAP, DDAB, 18PG, 14PA, and DMG-PEG-2000 were obtained from Avanti Polar Lipids. Cholesterol was from Sigma-Aldrich. B16F10 cells (CRL-6475) were purchased from ATCC (American Type Culture Collection, USA). DC 2.4 cells and B16F10-OVA (expressing model antigen, OVA, with a transmembrane domain) were kindly provided by the lab of Prof. Jonathan Schneck. Reporter lysis buffer and luciferin assay solution were purchased from Promega. All mRNA was purchased from (TriLink BioTechnologies). D-Luciferin was purchased from Gold Biotechnology, Alhydrogel® was purchased from InvivoGen.

### 5.5.2 Cell Culture and High-Throughput Screening for Transfection Studies

For monolayer culture studies, DC 2.4 cells were seeded into 96-well plates at a cell density of 10,000 cells per well one day prior to transfection. LNPs were pipetted into RPMI medium at a final concentration of 1 μg mL⁻¹ of mRNA. For example, 8 μL of an LNP suspension at 25 μg mL⁻¹ of mRNA was pipetted into the 200-μl culture media in each well. The transgene expression was analyzed following 24-h incubation. When characterizing luciferase as the reporter, cells were lysed by reporter lysis buffer (Promega) using two freeze-thaw cycles, with the lysate characterized by a luminometer upon addition of luciferin assay solution (Promega) against a standard curve generated using luciferase samples (Promega).

### 5.5.3 LNP Synthesis and Characterization

LNPs were synthesized by directly adding an organic phase containing the lipids to an aqueous phase containing the mRNAs in a 96-well plate or 1.5-mL microcentrifuge tubes for high-throughput screening. To prepare the organic phase, a mixture of Dlin-MC3 DMA, cholesterol (Sigma-Aldrich), DMG-PEG2000 (Avanti), and a helper lipid selected from a group consisting of DOTAP, DDAB, DOPE, DSPC, 14PA, 18PG (Avanti) were dissolved in ethanol. For SM-102 LNP preparation, a mixture of SM-102, DSPC, cholesterol and PEG-DMG at a molar ratio of 50:10:38.5:1.5 was prepared. To prepare the aqueous phase, corresponding mRNA (fLuc mRNA, GFP mRNA, mCherry mRNA, Cre mRNA, OVA mRNA, Trp2 mRNA, or Gp100 mRNA) was prepared in 25 mM magnesium acetate buffer (pH 4.0, Fisher). All mRNA samples were stored at −80° C. and thawed on ice before use. For in vitro screening, LNPs were incubated with cells without dialysis. For larger scale LNP production, the aqueous and ethanol phases prepared were mixed at a 3:1 ratio in a flash complexation (FNC) device using syringe pumps, Zhu et al., 2022, and purified by dialysis against DI water using a 100 kDa MWCO cassette (Fisher) at 4° C. for 24 h and were stored at 4° C. before injection. The size, polydispersity index and zeta potentials of LNPs were measured using dynamic light scattering (ZetaPALS, Brookhaven Instruments). Diameters are reported as the intensity mean average.

### 5.5.4 Animals and Primary Cells

All animal procedures were performed under an animal protocol approved by the Johns Hopkins Institutional Animal Care and Use Committee (protocol #MO21E193). Male and female C57BL/6 mice, 6-8 weeks of age, were purchased from the Jackson Laboratory. Male Ai9 mice, 6-8 weeks of age, were bred in Johns Hopkins Animal Facilities and randomly grouped. The mice were supplied with free access to pelleted feed and water. The pelleted feed generally contained 5% fiber, 20% protein, and 5-10% fat. The mice usually ate 4-5 g of pelleted feed (120 g per kg body weight) and drank 3-5 mL of water (150 mL per kg body weight) per day. The temperature of the mouse rooms was maintained at 18-26° C. (64-79° F.) at 30-70% relative humidity, with a minimum of 10 room air changes per hour. Standard shoebox cages with corncob as bedding were used to house the mice.

The LNPs were given through s.c. (right flank) or i.m. (right quadriceps) injection at a predetermined dose per mouse. The LNP suspensions were concentrated to 200 μg mL⁻¹ for s.c. injection or 400 μg mL⁻¹ for i.m. injection of mRNA by an Amicon Ultra-2 centrifugal filter unit with a MWCO of 100 kDa. For experiments in Ai9 mice, the Cre mRNA LNP formulations were prepared as described above and administered via s.c. or i.m. injections at a mRNA dose of 10 μg per mouse. After seven days, mice were sacrificed, and the draining lymph nodes were collected for flow cytometry analysis.

### 5.5.5 Antibodies, Cell Isolation and Staining for Flow Cytometry

Antibodies used in this study are: PE-Cyanine 7 anti-mouse CD40 (BioLegend #124622); PerCP-Cyanine 5.5 anti-mouse CD80 (BioLegend #104722); FITC, APC, Brilliant Violet 750 anti-mouse CD11c (BioLegend #117306, 117310, 117357); Brilliant Violet 421 anti-mouse CD86 (BioLegend #105032); PE anti-mouse SIINFEKL H-2 KB (ThermoFisher #12574382); FITC, Brilliant Violet 605, Brilliant Violet 421 anti-mouse CD45 (BioLegend #103108, 103140, 103134); APC anti-mouse CD3 (BioLegend #100236); FITC, APC, Brilliant Violet 750 anti-mouse CD8 (BioLegend #100706, 100712, BD Biosciences #747502); PerCP-Cyanine 5.5 anti-mouse CD4 (BioLegend #100540); PE anti-mouse IFN-γ (BioLegend #505808); Brilliant Violet 421 anti-mouse IL-4 (BioLegend #504120); PE-Cyanine 7 anti-mouse TNF-α (BioLegend #506324); and APC anti-mouse Granzyme B (BioLegend #396408). All antibodies were diluted at a ratio of 1:100 before use.

For isolation, re-stimulation and staining of spleen cells, the spleen was removed and minced using a sterile blade and homogenized in 250 μL of digestion medium (45 units μL⁻¹ collagenase I, 25 units μL⁻¹ DNase I and 30 units μL⁻¹ hyaluronidase). The suspension was transferred into a 15-mL tube containing 5-10 mL of digestion medium and then filtered through a 70-μm filter and washed once with PBS. Cells were pelleted at 300×g for 5 min at 4° C. and resuspended in 5 mL of red blood cell lysis buffer (BioLegend), and then incubated on ice for 5 min. Cells were then pelleted at 300×g for 5 min at 4° C. and washed for two times with PBS. The collected cells were then seeded into 12 well plate using RPMI-1640 media. Splenocytes were re-stimulated in vitro with OVA (InvivoGen Cat. vac-pova) and SIINFEKL peptide (InvivoGen Cat. vac-sin) (10 μg mL⁻¹ OVA and 2 μg mL⁻¹ SIINFEKL) for 12 h. After re-stimulation, cells were collected and centrifuged at 300×g for 5 min. Cell pellet was washed with staining buffer for 3 times and stained with antibodies against surface markers (total volume 100 μL) for 30 min in the dark at 4° C. The stained cells were washed twice with 1 mL of PBS, and then fixed and permeabilized using the fixation/permeabilization solution kit (BD Cat #555028). Then, cells were stained with anti-IFN-γ or other antibodies against intracellular cytokines. Flow data were acquired on Sony SH800 and analyzed using FlowJo software.

For isolation and staining of lymph node cells, isolated lymph nodes were mechanically digested through 70 μm nylon cell strainers to prepare single-cell suspensions. The cell suspension was washed once with PBS via centrifugation (300×g) for 5 min. Then, the cells were resuspended in 100 μL of staining buffer and stained with antibodies (total volume 100 μL) for 20 min in the dark at 4° C. The stained cells were washed twice with 1 mL of PBS and resuspended in 300 μl of staining buffer for flow cytometry analysis. Flow data were acquired on SH800 and analyzed using FlowJo software.

### 5.5.6 ELISpot Assay

Multiscreen filter plates (Millipore-Sigma #S2EM004M99) were coated with antibodies specific for IFN-γ (BD Biosciences #551881) and blocked following manufacturer's protocols. Then 1× 105 isolated splenocytes were plated per well and stimulated with SIINFEKL peptide (2 μg mL⁻¹ SIINFEKL) for 24 h. All tests were performed in duplicate or triplicate and included assay positive controls, as well as cells from a reference donor with known reactivity. Spots were visualized with mouse IFN-γ detection antibody (BD Biosciences #551881) followed by incubation with Streptavidin-HRP (BD Biosciences #557630) and AEC Substrate (BD Biosciences #551951). Plates were then sent to SKCCC Immune Monitoring Core for analysis.

### 5.5.7 Enzyme-Linked Immunosorbent Assay (ELISA)

For antibody detection, groups of C57BL/6 mice were immunized with different vaccines on days 0, 7 and 14. On day 21, 100 μL of blood sample was drawn from the tail vein, and levels of antigen-specific IgG in the serum were measured by ELISA. For ELISA, flat-bottomed 96-well plates (Nunc) were precoated with OVA protein at a concentration of 2 μg protein per well in 100 mM carbonate buffer (pH 9.6) at 4° C. overnight, which were then blocked with 10% fetal bovine serum (FBS) in PBS-Tween (PBS-T). Serum obtained from immunized animals were diluted 100 times in PBS-T (PBS-0.05% Tween), pH 7.4, and then in 4-fold serial dilution. The undiluted and diluted serum was added to the wells and incubated at 37° C. for 2 h. Horseradish peroxidase-conjugated goat anti-mouse IgG (Southern Biotech Associates, #1013-05) was used at a dilution of 1:5,000 in PBS-T-10% FBS for labeling. After adding the horseradish peroxidase substrates, optical densities were determined at a wavelength of 450 nm in an ELISA plate reader (Bio-Rad). A sample is considered as positive if its absorbance is twice as much as or higher than the absorbance of the negative control.

For cytokine detection, cell supernatant of BMDCs and splenocytes were obtained, and levels of IFN-γ, TNF-α, and IL-6 were measured by ELISA. Supernatant were diluted at 1:5. ELISA was performed using uncoated ELISA kits (Invitrogen) following the manufacturer's protocols. Optical densities were determined at a wavelength of 450 nm in an ELISA plate reader (Bio-Rad).

### 5.5.8 BMDC Isolation, Activation, and Antigen Presentation Assay

A mouse was sacrificed and transferred to a clean bench. The mouse was disinfected with 70% ethanol. The skin and muscle on the legs were carefully removed to separate the femur and tibia. The proximal and distal ends of each bone were cut with a pair of scissors. The bones were flushed with full medium (RPMI 1640, supplemented with 10% FBS and 1% penicillin/streptomycin). Two to three mL of medium was flushed from each side for each bone. The cell-containing medium was filtered through a 70 μm cell strainer, and the filtrate was collected. The cell suspension was centrifuged at 200×g for 10 min at room temperature, and the supernatant was discarded. The cells were resuspended in 10 mL full medium, and the cell concentration was determined. The cell suspension was diluted to a concentration of 3×10⁶ cells mL⁻¹. The cells were plated in ultra-low-attachment surface petri dishes by 10 mL per dish (100 mm×15 mm). Two mL of 40 ng mL⁻¹ GM-CSF was added in full medium to each well to a final GM-CSF concentration of 20 ng mL⁻¹. The cells were cultured in 37° C. and 5% carbon dioxide. Half of the GM-CSF-containing medium was replaced every 2 days. On day 6, nonadherent and loosely adherent immature dendritic cells were collected. The cell suspension was centrifuged at 200 g for 10 min at room temperature, and then the supernatant was discarded. The cells were plated at 5×10⁵ cells per well in a 24-well plate.

BMDCs were incubated with 1 μg mL⁻¹ OVA mRNA in various LNPs formulations or with PBS, free OVA (InvivoGen Cat. vac-pova), LPS (Sigma-Aldrich, Cat #L6529), or SIINFEKL peptide (InvivoGen Cat. vac-sin) in complete medium for 24 h at 37° C. with 5% CO₂; LPS+SIINFEKL peptide was used as a dendritic cell activation positive control. After co-incubation, BMDCs were collected, washed with FACS buffer (1% BSA, 10% FBS in PBS), and then stained on ice with fluorophore-labeled antibodies against CD45, CD11c, CD40, CD80, CD86, and SIINFEKL/H-2Kb monoclonal antibody.

### 5.5.9 Immunization and Tumor Therapy Experiments

Mice aged 6-8 weeks were injected subcutaneously with B16F10-OVA cells (1×10⁶ in prophylactic and depletion studies and 3× 10⁵ in therapeutic studies) or 3×10⁵ B16F10 melanoma cells into the right flank. In therapeutic studies, vaccinations began when tumor sizes were less than 50 mm³ (on day 4 after tumor inoculation). Animals were immunized by subcutaneous injection of different LNP formulations containing 10 μg OVA mRNA, mTrp2, or m Gp100 as described in the main text. A total of three doses were given. For combinatorial immunotherapy, at days 6, 13, 20 and an additional at day 27 for OVA-expressing melanoma after inoculation, some groups were intraperitoneally injected with 100 μg checkpoint inhibitor (anti-CTLA-4 mAb). Tumor growth was measured three times a week using a digital caliper and calculated as 0.5×length×width×width. Mice were euthanized when the tumor volumes reached 2,000 mm³.

### 5.5.10 Depletion Studies

Depletions of immune cells were done using antibodies against NK1.1 (clone PK136, BioXCell), CD3 (clone 145-2C11, BioXCell) and CD20 (clone MB20-11, BioXCell) at 200 μg i.p. every 4 days. All depletion antibodies dosing was initiated at 3 days before tumor inoculation and continued every 4 days.

### 5.5.11 Statistical Analysis

A two-tailed Student's t-test or a one-way analysis of variance (ANOVA) was performed when comparing two groups or more than two groups, respectively. Survival curves were compared using log-rank Mantel-Cox test and the stack of P values were corrected by Holm-Šídák method for multiple comparisons with alpha set to 0.05. Statistical analysis was performed using Microsoft Excel and Prism 8.0 (GraphPad). A difference is considered significant if P<0.05 (*P<0.05, **P<0.01, ***P<0.001, ****P<0.0001).

TABLE 7
Formulation details for the top 49 LNPs containing
mCherry mRNA in transfecting BMDCs

| | Molar % | | | | |
|---|---|---|---|---|---|
| Code | Hepler Lipid | Dlin-MC3 | Chol | DMG-PEG | N/P Ratio |
| A1 | 30.00 | 30.00 | 39.92 | 0.08 | 12 |
| A2 | 0.20 | 19.80 | 79.21 | 0.79 | 4 |
| A3 | 10.00 | 10.00 | 79.21 | 0.79 | 8 |
| A4 | 0.40 | 79.60 | 18.18 | 1.82 | 12 |
| A5 | 30.00 | 30.00 | 36.36 | 3.64 | 8 |
| B1 | 10.00 | 10.00 | 79.84 | 0.16 | 12 |
| B2 | 30.00 | 30.00 | 36.36 | 3.64 | 8 |
| B3 | 40.00 | 40.00 | 18.18 | 1.82 | 8 |
| C1 | 10.00 | 10.00 | 79.84 | 0.16 | 4 |
| C2 | 20.00 | 20.00 | 59.88 | 0.12 | 8 |
| C3 | 0.78 | 39.22 | 59.88 | 0.12 | 12 |
| C4 | 20.00 | 20.00 | 59.88 | 0.12 | 12 |
| C5 | 30.00 | 30.00 | 39.92 | 0.08 | 4 |
| C6 | 30.00 | 30.00 | 39.92 | 0.08 | 8 |
| C7 | 30.00 | 30.00 | 39.92 | 0.08 | 12 |
| C8 | 0.79 | 79.21 | 19.96 | 0.04 | 4 |
| C9 | 7.27 | 72.73 | 19.96 | 0.04 | 4 |
| C10 | 40.00 | 40.00 | 19.96 | 0.04 | 4 |
| C11 | 0.10 | 19.90 | 79.21 | 0.79 | 4 |
| C12 | 10.00 | 10.00 | 79.21 | 0.79 | 4 |
| C13 | 0.20 | 39.80 | 59.41 | 0.59 | 12 |
| C14 | 20.00 | 20.00 | 59.41 | 0.59 | 12 |
| D1 | 20.00 | 20.00 | 59.88 | 0.12 | 8 |
| D2 | 0.59 | 59.41 | 39.92 | 0.08 | 8 |
| D3 | 30.00 | 30.00 | 39.92 | 0.08 | 12 |
| D4 | 0.20 | 39.80 | 59.41 | 0.59 | 8 |
| D5 | 0.40 | 39.60 | 59.41 | 0.59 | 8 |
| D6 | 3.64 | 36.36 | 59.41 | 0.59 | 12 |
| D7 | 20.00 | 20.00 | 59.41 | 0.59 | 12 |
| D8 | 20.00 | 20.00 | 54.55 | 5.45 | 12 |
| E1 | 10.00 | 10.00 | 79.84 | 0.16 | 12 |
| E2 | 20.00 | 20.00 | 59.88 | 0.12 | 8 |
| E3 | 20.00 | 20.00 | 59.88 | 0.12 | 12 |
| E4 | 30.00 | 30.00 | 39.92 | 0.08 | 4 |
| E5 | 30.00 | 30.00 | 39.92 | 0.08 | 8 |
| E6 | 0.20 | 19.80 | 79.21 | 0.79 | 8 |
| E7 | 0.78 | 39.22 | 59.41 | 0.59 | 4 |
| E8 | 0.40 | 39.60 | 59.41 | 0.59 | 12 |
| E9 | 1.18 | 58.82 | 39.60 | 0.40 | 4 |
| E10 | 30.00 | 30.00 | 39.60 | 0.40 | 8 |
| E11 | 1.57 | 78.43 | 19.80 | 0.20 | 4 |
| E12 | 1.57 | 78.43 | 19.80 | 0.20 | 8 |
| F1 | 10.00 | 10.00 | 79.84 | 0.16 | 8 |
| F2 | 10.00 | 10.00 | 79.84 | 0.16 | 12 |
| F3 | 20.00 | 20.00 | 59.88 | 0.12 | 8 |
| F4 | 0.20 | 39.80 | 59.88 | 0.12 | 12 |
| F5 | 5.45 | 54.55 | 39.92 | 0.08 | 8 |
| F6 | 7.27 | 72.73 | 19.96 | 0.04 | 4 |
| F7 | 1.18 | 58.82 | 39.60 | 0.40 | 8 |

TABLE 8
Formulation details and particle sizes for the top
7 LNPs that showed >70% transfection efficiency

| | Mol % | | | | | | |
|---|---|---|---|---|---|---|---|
| | | | | DMG- | | Z-Average | |
| Code | Helper lipid | Dlin-MC3 | Chol | PEG | N/P Ratio | (nm) | PDI |
| C3 | 0.78 | 39.22 | 59.88 | 0.12 | 12 | 164.83 ± 3.38 | 0.29 ± 0.05 |
| C9 | 7.27 | 72.73 | 19.96 | 0.04 | 4 | 172.23 ± 5.42 | 0.18 ± 0.03 |
| C10 | 40.00 | 40.00 | 19.96 | 0.04 | 4 | 208.93 ± 33.85 | 0.32 ± 0.06 |
| D1 | 20.00 | 20.00 | 59.88 | 0.12 | 8 | 154.13 ± 2.79 | 0.27 ± 0.01 |
| D2 | 0.59 | 59.41 | 39.92 | 0.08 | 8 | 145.90 ± 0.99 | 0.26 ± 0.06 |
| D6 | 3.64 | 36.36 | 59.41 | 0.59 | 12 | 133.03 ± 2.27 | 0.28 ± 0.02 |
| F5 | 5.45 | 54.55 | 39.92 | 0.08 | 8 | 148.63 ± 1.28 | 0.21 ± 0.04 |

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Adams, D. et al., Patisiran, an RNAi therapeutic, for hereditary transthyretin amyloidosis. New England Journal of Medicine 379, 11-21 (2018).
Akinc, A. et al., The Onpattro story and the clinical translation of nanomedicines containing nucleic acid-based drugs. Nature Nanotechnology 14, 1084-1087 (2019).
Alameh, M. G. et al. Lipid nanoparticles enhance the efficacy of mRNA and protein subunit vaccines by inducing robust T follicular helper cell and humoral responses. Immunity 54, 2877-2892.e2877 (2021).
Alameh, M. G., Weissman, D. & Pardi, N. Messenger RNA-Based Vaccines Against Infectious Diseases. Curr Top Microbiol Immunol (2020).
Badrinath, S. et al. A vaccine targeting resistant tumours by dual T cell plus NK cell attack. Nature 606, 992-998 (2022).
Ballas, Z. K., et al., Induction of NK activity in murine and human cells by CpG motifs in oligodeoxynucleotides and bacterial DNA. Journal of Immunology 157, 1840-1845 (1996).
Bretscher, P. A. On the mechanism determining the TH1/TH2 phenotype of an immune response, and its pertinence to strategies for the prevention, and treatment, of certain infectious diseases. Scand J Immunol 79, 361-376 (2014).
Bretscher, P. On Analyzing How the Th1/Th2 Phenotype of an Immune Response Is Determined: Classical Observations Must Not Be Ignored. Front Immunol 10, 1234 (2019).
Cheng, Q., et al., Selective organ targeting (SORT) nanoparticles for tissue-specific mRNA delivery and CRISPR-Cas gene editing. Nature Nanotechnology 15, 313-320 (2020)
Cheng, X., Lee, R.J., The role of helper lipids in lipid nanoparticles (LNPs) designed for oligonucleotide delivery. Advanced Drug Delivery Reviews 99, 129-137 (2016).
Cullis, P.R., Hope, M. J., Lipid nanoparticle systems for enabling gene therapies. Molecular Therapy 25, 1467-1475 (2017).
Del Prete, G. The concept of type-1 and type-2 helper T cells and their cytokines in humans. Int Rev Immunol 16, 427-455 (1998).
Dilliard, S. A., Cheng, Q. & Siegwart, D. J. On the mechanism of tissue-specific mRNA delivery by selective organ targeting nanoparticles. Proc Natl Acad Sci USA 118 (2021)
Dobrowolski, C. et al. Nanoparticle single-cell multiomic readouts reveal that cell heterogeneity influences lipid nanoparticle-mediated messenger RNA delivery. Nat Nanotechnol 17 (8), 871-879 (2022).
Duarte, L. F. et al. Immune Profile and Clinical Outcome of Breakthrough Cases After Vaccination With an Inactivated SARS-COV-2 Vaccine. Front Immunol 12, 742914 (2021).
Guevara, M. L., Persano, F. & Persano, S. Advances in Lipid Nanoparticles for mRNA-Based Cancer Immunotherapy. Front Chem 8, 589959 (2020).
Han et al., An ionizable lipid toolbox for RNA delivery, Nature Communications, 12:7233 (2021).
Handumrongkul, C. et al., Durable multitransgene expression in vivo using systemic, nonviral DNA delivery. (2019).
Hartmann, G., Krieg, A. M., CpG DNA and LPS induce distinct patterns of activation in human monocytes. Gene Therapy 6, 893-903 (1999).
Hassett, K. J. et al. Impact of lipid nanoparticle size on mRNA vaccine immunogenicity. J Control Release 335, 237-246 (2021).
Hou, X., Zaks, T., Langer, R. & Dong, Y. Lipid nanoparticles for mRNA delivery. Nature Reviews Materials (2021).
Hu, Y., et al., Kinetic control in assembly of plasmid DNA/Polycation complex nanoparticles. ACS Nano 13, 10161-10178 (2019).
Hu, Y., et al., Size-controlled and shelf-stable DNA particles for production of lentiviral vectors. Nano Letters 21, 5697-5705 (2021).
Huang, Q., Zeng, J. & Yan, J. COVID-19 mRNA vaccines. J Genet Genomics 48, 107-114 (2021).
Huang, S., Zhu, Y., Zhang, L. & Zhang, Z. Recent Advances in Delivery Systems for Genetic and Other Novel Vaccines. Advanced Materials, 34 (46): e2107946 (2022).
International PCT patent application publication no. WO2020223323 for Compositionally Defined Plasmid DNA/Polycation Nanoparticles And Methods For Making The Same, to Mao et al., published Nov. 5, 2020.
International PCT Patent Application Publication No. WO2021252715 for Axisymmetric Confined Impinging Jet Mixer to Mittel et al., published Dec. 16, 2021.
J. Buck, J., et al., Lipid-based DNA therapeutics: hallmarks of non-viral gene delivery. ACS Nano 13, 3754-3782 (2019).
Jay, M. Charles, P. Eric, P., Targeting JAK/STAT signaling pathway in inflammatory diseases. Current Signal Transduction Therapy 4, 201-221 (2009).
Karlsson, J., et al., Poly(beta-amino ester) s as gene delivery vehicles: challenges and opportunities. Expert Opinion on Drug Delivery 17, 1395-1410 (2020).
Karmacharya, P., Patil, B. R. & Kim, J. O. Recent advancements in lipid-mRNA nanoparticles as a treatment option for cancer immunotherapy. J Pharm Investig 52, 415-426 (2022).
Kowalski, P. S., Rudra, A., Miao, L. & Anderson, D. G. Delivering the messenger: advances in technologies for therapeutic mRNA delivery. Mol Ther 27, 710-728 (2019).
Krieg, A. M., et al., CpG motifs in bacterial DNA trigger direct B-cell activation. Nature 374, 546-549 (1995).
Kulkarni, J. A., et al., Design of lipid nanoparticles for in vitro and in vivo delivery of plasmid DNA. Nanomedicine: Nanotechnology, Biology and Medicine 13, 1377-1387 (2017).
Kyriakidis, N. C., López-Cortés, A., González, E. V., Grimaldos, A. B. & Prado, E. O. SARS-COV-2 vaccines strategies: a comprehensive review of phase 3 candidates. NPJ Vaccines 6, 28 (2021).
Laczkó, D. et al. A single immunization with nucleoside-modified mRNA vaccines elicits strong cellular and humoral immune responses against SARS-COV-2 in mice. Immunity 53, 724-732.e727 (2020).
Lawrence, T., The nuclear factor NF-kappaB pathway in inflammation. Cold Spring Harb Perspect Biol 1, a001651-a001651 (2009).
Lederer, K. et al. SARS-COV-2 mRNA vaccines foster potent antigen-specific germinal center responses associated with neutralizing antibody generation. Immunity 53, 1281-1295.e1285 (2020).
Li, B. et al., An orthogonal array optimization of lipid-like nanoparticles for mRNA delivery in vivo. Nano Letters 15, 8099-8107 (2015).
Lindgren, G. et al. Induction of Robust B Cell Responses after Influenza mRNA Vaccination Is Accompanied by Circulating Hemagglutinin-Specific ICOS+PD-1+CXCR3+T Follicular Helper Cells. Front Immunol 8, 1539 (2017).
Liu, T., et al., NF-κB signaling in inflammation. Signal Transduct Target Ther 2, 17023 (2017).
Lokugamage, M. P., et al., Optimization of lipid nanoparticles for the delivery of nebulized therapeutic mRNA to the lungs. Nature Biomedical Engineering 5, 1059-1068 (2021).
Lozano-Rodríguez, R. et al. Cellular and humoral functional responses after BNT162b2 mRNA vaccination differ longitudinally between naive and subjects recovered from COVID-19. Cell Rep 38, 110235 (2022).
Miao, L. et al. Delivery of mRNA vaccines with heterocyclic lipids increases anti-tumor efficacy by STING-mediated immune cell activation. Nat Biotechnol 37, 1174-1185 (2019).
Miao, L., Zhang, Y. & Huang, L. mRNA vaccine for cancer immunotherapy. Mol Cancer 20, 41 (2021).
Mulligan, M. J. et al. Phase I/II study of COVID-19 RNA vaccine BNT162b1 in adults. Nature 586, 589-593 (2020).
Mulligan, R. C., The basic science of gene therapy. Science 260, 926-932 (1993).
Oberhardt, V. et al. Rapid and stable mobilization of CD8 (+) T cells by SARS-COV-2 mRNA vaccine. Nature 597, 268-273 (2021).
Oberli, M. A., et al., Lipid nanoparticle assisted mRNA delivery for potent cancer immunotherapy. Nano Letters 17, 1326-1335 (2017).
Painter, M. M. et al. Rapid induction of antigen-specific CD4 (+) T cells is associated with coordinated humoral and cellular immunity to SARS-COV-2 mRNA vaccination. Immunity 54, 2133-2142.e2133 (2021).
Pardi, N. et al. Characterization of HIV-1 nucleoside-modified mRNA vaccines in rabbits and rhesus macaques. Mol Ther Nucleic Acids 15, 36-47 (2019).
Pardi, N., Hogan, M. J., Porter, F. W. & Weissman, D. mRNA vaccines—a new era in vaccinology. Nat Rev Drug Discov 17, 261-279 (2018).
Patel, S. K. et al. Hydroxycholesterol substitution in ionizable lipid nanoparticles for mRNA delivery to T cells. J Control Release 347, 521-532 (2022).
Patel, S., Ryals, R. C., Weller, K. K., Pennesi, M. E. & Sahay, G. Lipid nanoparticles for delivery of messenger RNA to the back of the eye. J Control Release 303, 91-100 (2019)
Pfitzner, E., et al., The role of STATs in inflammation and inflammatory diseases. Curr Pharm Des 10, 2839-2850 (2004).
Pilkington, E. H. et al. From influenza to COVID-19: Lipid nanoparticle mRNA vaccines at the frontiers of infectious diseases. Acta Biomater 131, 16-40 (2021).
Polack, F. P. et al. Safety and Efficacy of the BNT162b2 mRNA Covid-19 Vaccine. N Engl J Med 383, 2603-2615 (2020).
Reichmuth, A. M., Oberli, M. A., Jaklenec, A., Langer, R. & Blankschtein, D. mRNA vaccine delivery using lipid nanoparticles. Ther Deliv 7, 319-334 (2016).
Richner, J. M. et al. Modified mRNA Vaccines Protect against Zika Virus Infection. Cell 168 (2017).
Sahin, U. et al. BNT162b2 vaccine induces neutralizing antibodies and poly-specific T cells in humans. Nature 595, 572-577 (2021).
Sahin, U. et al. COVID-19 vaccine BNT162b1 elicits human antibody and T (H) 1 T cell responses. Nature 586, 594-599 (2020).
Sahin, U., Karikó, K. & Türeci, Ö. mRNA-based therapeutics—developing a new class of drugs. Nat Rev Drug Discov 13, 759-780 (2014).
Salas, A., et al., JAK-STAT pathway targeting for the treatment of inflammatory bowel disease. Nature Reviews Gastroenterology & Hepatology 17, 323-337 (2020).
Sankaradoss, A. et al. Immune profile and responses of a novel dengue DNA vaccine encoding an EDIII-NS1 consensus design based on Indo-African sequences. Mol Ther 30, 2058-2077 (2022).
Scholz, C., Wagner, E., Therapeutic plasmid DNA versus siRNA delivery: Common and different tasks for synthetic carriers. Journal of Controlled Release 161, 554-565 (2012).
Sparwasser, T., et al., Bacterial DNA and immunostimulatory CpG oligonucleotides trigger maturation and activation of murine dendritic cells. European Journal of Immunology 28, 2045-2054 (1998).
Swingle, K. L. et al. Amniotic fluid stabilized lipid nanoparticles for in utero intra-amniotic mRNA delivery. J Control Release 341, 616-633 (2022).
Taniguchi, K., Karin, M., NF-κB, inflammation, immunity and cancer: coming of age. Nature Reviews Immunology 18, 309-324 (2018).
Turner, J. S. et al. SARS-COV-2 mRNA vaccines induce persistent human germinal centre responses. Nature 596, 109-113 (2021).
U.S. Patent Application Publication No. 20170042829, for Methods of Preparing Polyelectrolyte Complex Nanoparticles, to Mao et al., published Feb. 16, 2017.
U.S. Pat. No. 10,441,549 for Methods of Preparing Polyelectrolyte Complex Nanoparticles, to Mao et al., issued Oct. 15, 2019.
U.S. Pat. No. 11,229,609 for Compositions and methods for organ specific delivery of nucleic acids, to Cheng et al., published Jan. 25, 2022.
VanBlargan, L. A. et al. An mRNA vaccine protects mice against multiple tick-transmitted flavivirus infections. Cell Rep 25, 3382-3392.e3383 (2018).
Wang, Y., et al., Delivery of oligonucleotides with lipid nanoparticles. Advanced Drug Delivery Reviews 87, 68-80 (2015).
Wang, Z. et al. mRNA vaccine-elicited antibodies to SARS-COV-2 and circulating variants. Nature 592, 616-622 (2021).
Wei, T., et al., Systemic nanoparticle delivery of CRISPR-Cas9 ribonucleoproteins for effective tissue specific genome editing. Nature Communications 11, 3232 (2020).
Witzigmann, D. et al., Lipid nanoparticle technology for therapeutic gene regulation in the liver. Advanced Drug Delivery Reviews 159, 344-363 (2020).
Wouters, O. J. et al. Challenges in ensuring global access to COVID-19 vaccines: production, affordability, allocation, and deployment. Lancet 397, 1023-1034 (2021).
Xue, L. et al. Rational Design of Bisphosphonate Lipid-like Materials for mRNA Delivery to the Bone Microenvironment. J Am Chem Soc 144, 9926-9937 (2022).
Yew, N. S., et al., CpG-depleted plasmid DNA vectors with enhanced safety and long-term gene expression in vivo. Molecular Therapy 5, 731-738 (2002).
Yew, N. S., et al., Reduced inflammatory response to plasmid DNA vectors by elimination and inhibition of immunostimulatory CpG motifs. Molecular Therapy 1, 255-262 (2000).
Zhang, H. et al. Rational design of anti-inflammatory lipid nanoparticles for mRNA delivery. J Biomed Mater Res A 110, 1101-1108 (2022).
Zhang, Y. et al. Safety, tolerability, and immunogenicity of an inactivated SARS-CoV-2 vaccine in healthy adults aged 18-59 years: a randomised, double-blind, placebo-controlled, phase 1/2 clinical trial. Lancet Infect Dis 21, 181-192 (2021).
Zhang, Y., Sun, C., Wang, C., Jankovic, K. E. & Dong, Y. Lipids and Lipid Derivatives for RNA Delivery. Chemical Reviews 121 (20) (2021).
Zhu, Y. et al. Multi-step screening of DNA/lipid nanoparticles and co-delivery with siRNA to enhance and prolong gene expression. Nature Communications 13, 4282 (2022).
Zigmond, E., et al., Infiltrating monocyte-derived macrophages and resident kupffer cells display different ontogeny and functions in acute liver injury. The Journal of Immunology 193, 344-353 (2014).

Although the foregoing subject matter has been described in some detail by way of illustration and example for purposes of clarity of understanding, it will be understood by those skilled in the art that certain changes and modifications can be practiced within the scope of the appended claims.

1-50. (canceled)

51. A solid nanoparticle comprising a steroid, an ionizable cationic lipid, a helper lipid, a PEGylated lipid, and a nucleic acid payload comprising one or more nucleic acids, wherein the nanoparticle comprises:
a molar ratio of the steroid to the PEGylated lipid of between about 10 to about 900;
a molar ratio of the ionizable cationic lipid to the helper lipid of between about 1 to about 200;
a total percentage of the ionizable lipid and the helper lipid between about 20% to about 80%; and
an N to P ratio between about 2 to about 14.

52. The solid nanoparticle of claim 51, wherein the steroid comprises a sterol.

53. The solid nanoparticle of claim 52, wherein the sterol comprises cholesterol.

54. The solid nanoparticle of claim 51, wherein the ionizable cationic lipid comprises Dlin-MC3-DMA.

55. The solid nanoparticle of claim 51, wherein the helper lipid is selected from a cationic lipid, a zwitterionic lipid, and an anionic lipid.

56. The solid nanoparticle of claim 55, wherein:
(a) the cationic lipid is selected from 1,2-dioleoyl-3-trimethylammonium-propane (DOTAP) and dimethyldioctadecyl ammonium (DDAB); and/or
(b) the zwitterionic lipid is selected from 1,2-dioleoyl-sn-glycero-3-phosphoethanolamine (DOPE), 2-((2,3-bis(oleoyloxy) propyl)dimethylammonio) ethyl ethyl phosphate (DOCPe), and 1,2-distearoyl-sn-glycero-3-phosphocholine (DSPC).

57. The solid nanoparticle of claim 51, wherein the anionic lipid comprises a phospholipid.

58. The solid nanoparticle of claim 57, wherein the phospholipid is selected from 1,2-dimyristoyl-sn-glycero-3-phosphate (14PA) and 1-stearoyl-2-oleoyl-sn-glycero-3-phospho-(1′-rac-glycerol) (18PG).

59. The solid nanoparticle of claim 51, wherein the PEGylated lipid comprises dimyristoyl glycerol (DMG)-polyethyleneglycol (PEG) 2000 (DMG-PEG2000).

60. The solid nanoparticle of claim 51, wherein the one or more nucleic acids are selected from plasmid DNA (pDNA), siRNA, mRNA, and combinations thereof.

61. The solid nanoparticle of claim 60, wherein the siRNA comprises an anti-inflammatory siRNA.

62. The solid nanoparticle of claim 51, wherein the nanoparticle has a size smaller than about 400 nm.

63. A method for delivering one or more nucleic acids to a liver of a subject, the method comprising administering to a subject in need of treatment thereof a solid nanoparticle of claim 51.

64. The method of claim 63, wherein the one or more nucleic acids are selected from plasmid DNA (pDNA), siRNA, and combinations thereof.

65. The method of claim 64, wherein the siRNA comprises an anti-inflammatory siRNA.

66. The method of claim 65, wherein:
(a) the anti-inflammatory siRNA targets a transcription factor selected from signal transducer and activator of transcription (STAT), and nuclear factor kappa-light-chain-enhancer of activated B cells (NF-κB);
(b) administration of the anti-inflammatory siRNA reduces inflammation-induced gene silencing;
(c) an expression duration of the pDNA when co-administered with the anti-inflammatory siRNA is longer than an expression duration of the pDNA when administered alone;
(d) an expression level of the pDNA when co-administered with the anti-inflammatory siRNA substantially similar to an expression level of the pDNA when administered alone; and/or
(e) reducing a level within the liver of one or more of signal transducer and activator of transcription (STAT), nuclear factor kappa-light-chain-enhancer of activated B cells (NF-κB), one or more infiltrating inflammatory monocytes, and one or more apoptotic cells.

67. The method of claim 65, wherein the one or more infiltrating inflammatory monocytes are selected from CD45⁺ and CD11b⁺ cells.

68. The method of claim 63, comprising treating one or more diseases or disorders of the liver.

69. The method of claim 68, wherein the one more diseases or disorders of the liver are selected from a genetic liver disease and an inflammatory liver disease.

70. The method of claim 69, wherein the one or more disease or disorders of the liver is selected from haemophilia B, haemophilia A, ornithine transcarbamylase (OTC) deficiency, phenylketonuria, acute intermittent porphyria, methylmalonic acidemia, familial hypercholesterolemia, Fabry, MPS type VI, Gangliosidosis GM1, Danon disease, GSD1a Von Gierke, Wilson's disease, Crigler-Najjar, primary hyperoxaluria type 1, and combinations thereof.

71. The method of claim 63, wherein the method for delivering the one or more nucleic acids to a liver of a subject is selected from intravenous (i.v.) injection, oral, subcutaneous, and inhalation delivery.

72. A method for preparing a solid nanoparticle of claim 51, the method comprising:
(a) preparing an organic phase by solubilizing a mixture of a steroid, an ionizable cationic lipid, a helper lipid, a PEGylated lipid in a polar, protic solvent at a predetermined molar ratio;
(b) preparing an aqueous phase by dissolving one or more nucleic acids in an aqueous buffer; and
(c) combining the organic phase and the aqueous phase to form the solid nanoparticle.

73. The method of claim 72, wherein:
(a) the polar, protic solvent comprises a C₁-C₄ alcohol; and/or
(b) the aqueous buffer comprises a magnesium acetate buffer.

74. The method of claim 72, further comprising mixing the organic phase and the aqueous phase in a flash nanocomplexation (FNC) device.

75. The method of claim 74, further comprising mixing the organic phase and the aqueous phase at an about 3:1 ratio.

76. The method of claim 72, further comprising dialyzing the solid nanoparticle against deionized water.

77. A method for stimulating a Type-1 T helper (Th1) and/or a Type-2 T helper (Th2) response in vivo, the method comprising administering a solid nanoparticle of claim 51.

78. The method of claim 77, wherein:
the steroid comprises cholesterol;
the ionizable cationic lipid comprises DLin-MC3-DMA;
the PEGylated lipid comprises DMG-PEG2000;
the nucleic acid is a mRNA; and
the helper lipid is selected from 1,2-dioleoyl-3-trimethylammonium-propane (DOTAP), dimethyldioctadecyl ammonium (DDAB), 1,2-dioleoyl-sn-glycero-3-phosphoethanolamine (DOPE), DSPC, 1,2-dimyristoyl-sn-glycero-3-phosphate (14PA), and 1-stearoyl-2-oleoyl-sn-glycero-3-phospho-(1′-rac-glycerol) (18PG).

79. The method of claim 78, wherein the solid nanoparticle comprises:
a combined molar percentage of DLin-MC3-DMA and helper lipid ranging from about 20% to about 80%;
a weight ratio of cholesterol to DMG-PEG2000 ranging from about 10 to about 500;
a weight ratio of DLin-MC3-DMA to helper lipid ranging from about 1 to about 200; and
a molar ratio of chargeable groups in the ionizable lipid to phosphate groups in mRNA (N/P ratio) ranging from about 4 to about 12.

80. The method of claim 79, wherein the solid nanoparticle comprises:
(a) about 30 molar % DOPE, about 30 molar % DLin-MC3-DMA, about 40 molar % cholesterol, about 0.40 molar % DMG-PEG2000, and a N/P ratio of about 4;
(b) about 7 molar % DSPC, about 70 molar % DLin-MC3-DMA, about 20 molar % cholesterol, about 0.04 molar % DMG-PEG2000, and a N/P ratio of about 4; or
(c) about 5 molar % 18PG, about 55 molar % DLin-MC3-DMA, about 40 molar % cholesterol, about 0.40 molar % DMG-PEG2000, and a N/P ratio of about 12.

81. The method of claim 77, wherein the method induces an immune response in Th1 only, in Th2 only, or in both Th1 and Th2.

82. A method for treating a disease, disorder, or condition in subject, the method comprising administering a therapeutically effective dose of a solid nanoparticle of claim 51 to a subject in need of treatment thereof.

83. The method of claim 82, wherein the disease is selected from a cancer or an infection.

84. The method of claim 83, wherein the cancer is selected from basal cell carcinoma, bladder cancer, breast cancer, cervical cancer, colorectal cancer, endometrial cancer, esophageal carcinoma, gastric cancer, head and neck cancer, hepatocellular carcinoma, Hodgkin's lymphoma, malignant pleural mesothelioma, Merkel cell carcinoma, metastatic melanoma, non-small cell lung cancer, renal cell carcinoma, small cell lung cancer, squamous cell carcinoma, and urothelial carcinoma.

85. The method of claim 83, wherein the infection comprise a viral infection.

86. The method of claim 85, wherein the viral infection is selected from a coronavirus infection, a Zika virus infection, influenza, a flavivirus infection, and a human immunodeficiency virus (HIV) infection.

87. The method of claim 77, further comprising administering the solid nanoparticle with one or more immune checkpoint inhibitors.

88. The method of claim 87, wherein the immune checkpoint inhibitor is selected from a CTLA-4 inhibitor, a PD-1 inhibitor, and a PD-L1 inhibitor.

89. The method of claim 87, wherein the one or more immune checkpoint inhibitors is selected from Ipilimumab, Nivolumab, Pembrolizumab, Atezolizumab, Avelumab, Durvalumab, and Cemiplimab.

90. A vaccine comprising the solid nanoparticle of claim 51.

91. The vaccine of claim 90, wherein the vaccine is a cancer vaccine or an anti-viral vaccine.
